(19) United States
(12) Patent Application Publication
(10) Pub. No.: US 2023/0235369 A1
(43) Pub. Date: Jul. 27, 2023

(54) Heterotrophic production of essential long-chain polyunsaturated lipids (LCPUFA) in Auxenochlorella protothecoides

(71) Applicant: PHYCOIL BIOTECHNOLOGY INTERNATIONAL, INC., Fremont, CA (US)

(72) Inventors: Riyaz Bhat, Fremont, CA (US); Jeffrey Moseley, Fremont, CA (US); Jane KIM, Fremont, CA (US); Chungsoon IM, Fremont, CA (US)

(21) Appl. No.: 18/064,390

(22) Filed: Dec. 12, 2022

Related U.S. Application Data
(60) Provisional application No. 63/288,041, filed on Dec. 10, 2021.

(51) Int. Cl. C12P 7/6432; C12N 1/12; C12N 9/02; C12N 15/63

(52) U.S. Cl. CPC C12P 7/6432; C12N 1/125; C12N 9/0071; C12Y 114/19001; C12N 15/63

(57) ABSTRACT

Microalgal mutant to produce high-value essential LCPUFA oils including eicosadienoic acid (EDA), dihomo-γ-linoleic acid (DGLA), arachidonic acid (ARA), and eicosapentaenoic acid (EPA) in various ratios in are provided.

### CROSS-REFERENCE TO RELATED APPLICATION

This application claims the benefit of U.S. Provisional Application No. 63/288,041 filed on Dec. 10, 2021, the disclosures of which are herein incorporated by reference in their entirety.

The present invention describes microalgae as having the ability to produce high-value essential long-chain poly polyunsaturated fatty acids (LCPUFA) oils eicosadienoic acid (EDA), dihomo-γ-linoleic acid (DGLA), arachidonic acid (ARA), and eicosapentaenoic acid (EPA) in various ratios, and a method of extracting the oil using the microalgae, and method for preparing said essential LCPUFA oils, compared to wild-type microalgae, or previously disclosed strain such as Auxenochlorella protothecoides with in-house strain designation as PB5 (disclosed in U.S. application Ser. No. 17/519,854).

The present invention also relates to the production of oils, fuels, and oleochemicals made from microorganisms. In particular, the disclosure relates to oil-bearing microalgae, methods of cultivating them for the production of useful compounds, including lipids, fatty acid esters, fatty acids, aldehydes, alcohols, and alkanes, and methods and reagents for genetically altering them to improve production efficiency and alter the type and composition of the oils produced by them.

### BACKGROUND ART

The oleaginous Trebouxiophyceae alga, Auxenochlorella protothecoides, with in-house strain designation as PB5, stores copious amounts of triacylglyceride (TAG) oil under conditions where the nutritional carbon supply is in excess, but cell division is inhibited due to limitation of other essential nutrients. Heterotrophically grown Auxenochlorella strains also degrade chlorophyll and down-regulate photosynthesis but maintain significant levels of the yellow carotenoids—lutein and zeaxanthin. Bulk biosynthesis of fatty acids with carbon chain lengths up to C18 occurs in the plastids; fatty acids are then exported to the endoplasmic reticulum for eventual incorporation into triacyl glycerides (TAGs; see FIG. 1). Fatty acids produced in the plastids, however, are not always immediately available for TAG biosynthesis and may undergo further modification, including channeling through phospholipids, before being incorporated into TAGs. Lysophosphatidylcholine acyltransferase (LPCAT) enzymes play a central role in the acyl editing of phosphatidylcholine (PC) in the phospholipid membranes. LPCAT enzymes work in both forward and reversible reaction modes. In the forward mode, they are responsible for the channeling of oleic acid (C18:1n-9) into PC for subsequent desaturation by fatty acid desaturases (FAD; FIG. 1). In the reverse reaction mode, they transfer oleic acid esterified to the PC back into the acyl CoA pool. There are at least two possible routes whereby acyl residues from PC are incorporated into the TAG. First, the DAG moiety of PC can be liberated (by hydrolysis) by the reversible action of CDP-choline: 1,2-sn-diacylglycerol choline phosphotransferase (CPT or DAG-CPT), thus becoming available for TAG assembly by diacylglycerol acyltransferase (DGAT). The second route involves the activity of an enzyme known as phosphatidylcholine: 1,2-sn-diacylglycerol choline phosphotransferase (PDCT). Like CPT, the PDCT mediates a symmetrical interconversion between phosphatidylcholine (PC) and diacylglycerol (DAG), thus enriching PC-modified fatty acids—C18:2n-6 and C18:3n-3—in the DAG pool prior to forming TAG. C18:2n-6 and C18:3n-3 can be further elongated and desaturated to produce essential PUFA's like eicosadienoic acid (EDA), dihomo-γ-linoleic acid (DGLA), arachidonic acid (ARA), eicosapentaenoic acids (EPA). Alternatively, C18:1 n-9 can also be elongated directly to produce very long-chain fatty acids (VLCFAs).

Neutral lipids are stored in large cytoplasmic organelles called lipid bodies until environmental conditions change to favor growth, whereupon they are rapidly mobilized to provide energy and carbon molecules for anabolic metabolism. Wild-type A. protothecoides storage lipid is comprised mainly of oleic (˜68%), palmitic (˜12%), and linoleic (˜13%) acids, with minor amounts of stearic, myristic, α-linolenic (ALA), and palmitoleic acids. This fatty acid profile results from the relative activities and substrate affinities of the enzymes of the endogenous fatty acid biosynthetic pathway. A. protothecoides is amenable to manipulation of fatty acid and lipid biosynthesis using molecular genetic tools, enabling the production of oils with fatty acid profiles that are significantly different from the wild-type composition. Similarly, the carotenoid and phytosterol profile of the lipid fraction can be altered by genetic engineering of terpenoid biosynthesis pathways.

Auxenochlorella protothecoides is publicly available to purchase through the University of Texas (UTEX) Culture Collection (UTEX catalog number 250) through its webpage www.utex.org.

The inventors of the present invention have demonstrated efficient transformation and facile nuclear gene targeting via homologous recombination in A. protothecoides PB5 in earlier examples (see U.S. application Ser. No. 17/519,854). In the following examples, the inventors of the present invention leverage their ability to perform gene knockouts and knock-ins and regulatory element hijacking to produce algal oils with significantly modified polyunsaturated fatty acid profiles.

### BRIEF SUMMARY OF THE INVENTION

The inventors of the present invention have developed the Trebouxiophyceae alga, Auxenochlorella protothecoides (PB5), as a biotechnology platform for the heterotrophic production of high-value lipids, carotenoids, terpenoids, and other compounds. Specifically, the inventors of the present invention demonstrate that the combinatorial expression of Arabidopsis thaliana lysophosphatidylcholine acyltransferase (At-LPCAT1; Accession No: NP_172724) and phosphatidylcholine: 1,2-sn-diacylglycerol choline phosphotransferase (At-PDCT, Accession No: NP_566527), delta-9 elongase from Euglena gracilis (Eg-delta-9FAE, Accession number: CAT16687), Isochrysis galbana (Ig-delta-9FAE, Accession No's: AF390174_1 and ADD51571) and Pavlova pinguis (Ppin-delta-9FAE; Accession No: ADN94475), delta-8 desaturases from I. galbana (Ig-FADdelta-8; Accession No: AFB82640), Pavlova salina (Ps-FADdelta-8; Accession No: A4KDP1.1), Pavlovales sp. CCMP2436, Diacronema lutheri (DI-FADdelta-8; Accession No: KAG8471305), Capsaspora owczarzaki (Cowc-FADdelta-8; Accession No: KAG8471305.1), Perkinus marinus (Pmari-FADdelta-8; Accession No: ABF58684.1), and Perkinus olseni (Pols-FADdelta-8; Accession No: KAF4696203.1), delta-5 desaturase from Phaeodactylum tricornutum (Pt-FADdelta-5; Accession No: AAL92562), and delta-17 desaturases from Pythium aphanidermatum (Pa-FADdelta-17; Accession No: AOA52182), Phytophthora sojae (Ps-FADdelta-17; Accession No: FW362213) and Saprolegnia diclina (Sd-FADdelta-17; Accession No: Q6UB73) results in efficient channeling of C18:1n-9 through phospholipids (via action of At-LPCAT1 and At-PDCT) where they are desaturated by endogenous fatty acid desaturase delta-12 (FADdelta-12) for either direct incorporation into DAGs and TAGs or for elongation to C20:2n-6 (eicosadienoic acid, EDA) by action of delta-9 elongases, further desaturation of EDA first into C20:3n-6 (dihomo-γ-linoleic acid or DGLA) via action of FADdelta-8, followed by 20:4n-6 (Arachidonic acid or ARA) via action of FADdelta-5, and then into C20:5n-3 (Eicosapentaenoic acid; EPA) via action of FADdelta-17 desaturases.

Both CDP-choline: 1,2-sn-diacylglycerol choline phosphotransferase (CPT and/or DAG-CPT) and phosphatidylcholine: 1,2-sn-diacylglycerol choline phosphotransferase (PDCT) enzyme activities, described in FIG. 1, maintain a proper C18:2n-6 and C18:3n-3 ratio in phospholipid membranes and ensure that any unusual fatty acids synthesized therein (including any excess of C18:2n-6 and C18:3n-3) are properly channeled out for incorporation into DAGs and eventually TAGs thus maintaining the functional integrity of these membranes. Since A. protothecoides and A. thaliana do not produce significant amounts of fatty acids beyond C18:2n-6 and C18:3n-3, the inventors of the present invention envisage that the CPT/DAG-CPT and PDCT enzyme activities in these organisms are fairly limited in efficient channeling of LCPUFAs into DAGs and TAGs. To boost this channeling, the applicant of the present application has identified CPT or EPT (ethanolamine choline phosphotransferase), DAG-CPT (CDP-choline: 1,2-sn-diacylglycerol choline phosphotransferase), and PDCT-like enzyme activities from a proprietary organism (Oblongichytrium sp. in-house strain designation with PB75), that produces significant amounts of LCPUFAs via elongase-desaturase pathway. The genes corresponding to these enzymes were codon-optimized to reflect PB5 codon usage, have been expressed in PB5, and significantly improved the synthesis and accumulation of various essential LCPUFAs in our host. Transformations, cell culture, lipid production, and quantification were all carried out as previously described.

The present invention provides a microalgal host for the production of high-value essential long-chain poly polyunsaturated fatty acids (LCPUFA) oils in various ratios thereof. The microalgae is an Auxenochlorella protothecoides incorporating a number of genetic elements and modifications that make it uniquely attractive for LCPUFA production.

Accordingly, an object of the present invention is to provide a microalgal mutant having the ability to produce high-value essential LCPUFA oils including eicosadienoic acid (EDA), dihomo-γ-linoleic acid (DGLA), arachidonic acid (ARA), and eicosapentaenoic acid (EPA) in various ratios. More specifically, the invention provides a recombinant Auxenochlorella protothecoides for the production of long-chain polyunsaturated fatty acid comprising a combination of at least one gene encoding elongase and at least one gene encoding desaturases.

In another embodiment, the elongase is delta-9 elongase (delta-9FAE).

In another embodiment, the desaturase is at least one gene selected from a group consisting of:

a) gene encoding Δ8 desaturase;

b) gene encoding Δ5 desaturase; and

c) gene encoding Δ17 desaturase.

In another embodiment the invention provides a recombinant Auxenochlorella protothecoides for the production of long-chain polyunsaturated fatty acid further comprising at least one gene selected from a group consisting of:

a) gene encoding LPCAT;

b) gene encoding LPAAT;

c) gene encoding Cytochrome b5;

d) gene encoding PDCT; and

e) gene encoding CPT.

Another object of the present invention is to provide a method for increasing the pool of available carbon (in the form of Malonyl CoA) in the cytosol by upregulating the Homomeric ACCase gene by replacing its promoter with a stronger heterologous promoter to sustain LCPUFA synthesis in Auxenochlorella protothecoides.

Another object of the present invention is to provide a method for the production of a microbial oil comprising long-chain polyunsaturated fatty acid comprising:

a) introducing a combination of at least one nucleic acid sequence which encodes elongases and at least one nucleic acid sequence which encodes desaturases into Auxenochlorella protothecoides to prepare the recombinant Auxenochlorella protothecoides; and

b) culturing the recombinant Auxenochlorella protothecoides to produce long-chain polyunsaturated fatty acids; and

Another object of the present invention is to provide pairwise alignments of various protein sequences that encode lysophosphatidylcholine acyltransferase (LPCAT) from Arabidopsis thaliana, B. rapa and B. napus and phosphatidylcholine: 1,2-sn-diacylglycerol choline phosphotransferase (PDCT) from A. thaliana, specific choline/ethanolamine phosphotransferase (CPT/EPT), diacylglycerol cholinephosphotransferase (DAG-CPT), phosphatidyl-choline: 1,2-sn-diacylglycerol cholinephosphotransferase (PDCT)-like enzymes from a Phycoil proprietary strain PB75 (Oblongichytrium sp., as shown in FIG. 2.), delta-9 elongase from Euglena gracilis, Isochrysis galbana and Pavlova pinguis, delta-8 desaturases from E. gracilis, Perkinus marinus, I. galbana, P olseni, Mortierella sp. NVP85, Mortierella alpina, Diacronema lutheri, Pavlovales sp. CCMP2436, Pavlova salina, and Capsaspora owczarzaki, delta-5 desaturases from Phaeodactylum tricornutum, Dictyostelium discoideum, M. alpina, C. elegans, Oblongichytrium sp. SEK 347, Euglena gracilis, Parietochloris incisa, and Thalassiosira pseudonana CCMP1335, and delta-17 desaturases from Pythium aphanidermatum, Phytophthora sojae, Phytophthora ramorum, and Saprolegnia diclina.

Another object of the present invention is to provide a recombinant nucleic acid comprising a Auxenochlorella protothecoides codon optimized sequence that encodes Arabidopsis thaliana lysophosphatidyl-choline acyltransferase (LPCAT) and phosphatidyl-choline: 1,2-sn-diacylglycerol cholinephosphotransferase (PDCT), delta-9 elongase from Euglena gracilis, Isochrysis galbana and Pavlova pinguis, delta-8 desaturases from I. galbana, Pavlova salina, delta-5 desaturase from Phaeodactylum tricornutum, and delta-17 desaturases from Pythium aphanidermatum, Phytophthora sojae and Saprolegnia diclina, Mortierella alpina lysophosphatidic acid acyltransferase (LPAAT), Arabidopsis thaliana cytochrome b5 (Cytb5), PB75 (Oblongichytrium sp.) choline phosphotransferase (CPT) that kick starts LCPUFA biosynthesis and enhance accumulation of LCPUFA in Auxenochlorella protothecoides.

Another object of the present invention is to provide a recombinant Auxenochlorella protothecoides transformed with a recombinant nucleic acid provided herein.

Another object of the present invention is to provide microbial oil comprising long-chain polyunsaturated fatty acid prepared by the above production method.

Another object of the present invention is to provide a composition comprising the above microalgal mutant, a culture thereof, or the above oil.

### Advantageous Effects

The present application shows that when using a microalgal mutant in which Auxenochlorella protothecoides PB5 microalga genes are knocked out or knocked in by various recombination methodologies, a high-value essential LCPUFA oil including eicosadienoic acid (EDA), dihomo-γ-linoleic acid (DGLA), arachidonic acid (ARA), and eicosapentaenoic acid (EPA), in various ratios compared to that of wild type thereof and Auxenochlorella protothecoides PB5 can be effectively extracted.

### DESCRIPTION OF THE DRAWING

FIG. 1 shows a diagram of Fatty acid biosynthesis and multiple fates of C18:1n-9 following its exit from the plastid in algae and higher plants. After becoming associated with coenzyme A (CoA), C18:1 n-9 enters the ER and is either incorporated directly into TAGs [via the Kennedy pathway, involving Lysophosphatidic acid acyltransferase (LPAAT), Diglyceride acyltransferase (DGAT), and Glycerol-3-phosphate acyltransferase (GPAT) enzyme activities] or is further desaturated via the Lands cycle pathway (involving LPCAT, CPT, and PDCT enzyme activities) to produce C18:2n-6 and C18:3n-3 which can be further elongated to produce eicosadienoic acid (EDA), dihomo-γ-linoleic acid (DGLA), arachidonic acid (ARA) and eventually eicosapentaenoic acids (EPA). C18:1 n-9 can also be elongated directly to produce very long-chain fatty acids (VLCFAs).

FIG. 2A-H show alignments of proteins encoding LPCAT CPT DAG-CPT PDCT delta-9 elongase, FADdelta-8, FAD-delta-5, and FADdelta-17 activities from various organisms. 2A—Protein alignment displaying conservation of LPCAT (also known as membrane-bound O-acyltransferase or MBOAT) proteins from A. thaliana (NP_172724 and NM_104983), Brassica rapa (XM_009150328), and B. napus (XM_013887149 and XM_048758019).

2B—Protein alignment displaying conservation and divergence of CPT (CPT/EPT 1 PB75_006534-T1 and CPT/EPT 1 PB75_009271-T1), and DAG-CPT (CPT/DAG-CPT/EPT 1 PB75_005318-T1) like proteins from a Phycoil proprietary strain PB75 against known CPT enzymes from Phytophthora infestans (XM_002900684).

2C—Protein alignment displaying divergence of A. thaliana PDCT (NP_566527) and a PDCT-like protein from Phycoil proprietary organism PB75 (PB75-PDCT, PB75_012102-T1).

2D—Protein alignment displaying conservation of delta-9 elongases from E. gracilis (CAT16687), I. galbana (ADD51571 and AF390174_1), and P. pinguis (ADN94475).

2E—Protein alignment displaying conservation and divergence of FAD delta-8 desaturases from E. gracilis (AAD45877.1 and ADD51570.1), Perkinus marinus (ABF58684), I. galbana (AFB82640), P. olseni (KAF4696203 and KAF4740840), Mortierella sp. NVP85 (KAF9358687), Mortierella alpina (KAF9932301), Diacronema lutheri (KAG8471305), Pavlovales sp. CCMP2436, Pavlova salina (A4KDP1.1), and Capsaspora owczarzaki (XP_004346669).

2F—Protein alignment displaying conservation and divergence of FAD delta-5 desaturases from Phaeodactylum tricornutum (AAL92562 and XP_002182858), Dictyostelium discoideum (AB022097), Mortierella alpina (AF054824 and 074212), Caenorhabditis elegans (AF078796), Oblongichytrium sp. SEK 347 (BAG71007), Euglena gracilis (CBH30563), Parietochloris incisa (GU390533), Thalassiosira pseudonana CCMP1335 (XP_002296867).

2G—Protein alignment displaying conservation of FAD delta-17 desaturases from Pythium aphanidermatum (AOA52182), Phytophthora sojae (FW362213), Phytophthora ramorum (FW362214), Saprolegnia diclina (Q6UB73)

2H—Protein alignment displaying conservation of Cytochrome b5 from A. thaliana (AAC04491.1, BAA74839.1, BAA74840.1, AAC69922.1, NP_173958, AAB71978.1), G. max (XP_028236170, NP_001236501, KAH1240713, NP_001236891, XP_028218521, NP_001239968, NP_001238196, NP_001236788), and Vernicia fordii (AAT84458, AAT84459, AAT84460)

2I— Protein alignment displaying conservation of LPAAT-like enzymes from L. douglasii (CAA86877 and Q42870), Mortierella alpina (KAF9934294 and KAF9941528), and various candidate enzymes from a Phycoil proprietary strain PB75 (PB75_007410-T1, PB75_010969-T1, PB75_011464-T1, PB75_010418-T1, PB75_004141-T1, PB75_008188-T1, and PB75_008188-T1)

FIGS. 3A and B show the nucleotide sequence of the transforming DNA contained in plasmid pPB0177 (SEQ NO.: 1).

FIG. 4 shows the nucleotide sequence of codon-optimized Ig-ASE1 fatty acid elongase in pPB0178 (SEQ NO.: 2).

FIG. 5 shows the nucleotide sequence of codon-optimized Ig-ASE2 fatty acid elongase in pPB0179 (SEQ NO.: 3).

FIG. 6 shows the nucleotide sequence of codon-optimized Ppin-delta-9FAE fatty acid elongase in pPB0180 (SEQ NO.: 4).

FIGS. 7A and B show the nucleotide sequence of the transforming DNA contained in plasmid pPB0238 (SEQ NOS.: 5).

FIG. 8 shows the nucleotide sequence of codon-optimized Ps-FADdelta-8 fatty acid desaturase contained in plasmid pPB0239 (SEQ NO.: 6).

FIGS. 9A and B show the nucleotide sequence of the transforming DNA contained in plasmid pPB0234 (SEQ NO.: 7).

FIG. 10 shows the nucleotide sequence of the ApAMT1-At-PDCT-ApPGK1 cassette contained in plasmid pPB0214 (SEQ NO.: 8).

FIGS. 11A and B show the nucleotide sequence of the ApAMT1-At-PDCT-ApPGK1: ApAMT2v1-At-LPCAT1-ApSAD21 cassettes contained in plasmid pPB0222 (SEQ NO.: 9).

FIGS. 12A and B show the nucleotide sequence of the transforming DNA contained in plasmid pPB0265 (SEQ NO.: 10).

FIG. 13 shows the nucleotide sequence of the ApSAD2v1-Ig-FADdelta-8-ApSAD2v1 UTR cassette contained in plasmid pPB0266 (SEQ NO.: 11).

FIG. 14 shows the nucleotide sequence of the ApSAD2v1-Ps-FADdelta-8-ApSAD2v1 UTR cassette contained in plasmid pPB0267 (SEQ NO.: 12).

FIG. 15A-C show the nucleotide sequence of the transforming DNA contained in plasmid pPB0274 (SEQ NO.: 13).

FIG. 16 shows the nucleotide sequence of codon-optimized Pt-FADdelta-5 fatty acid desaturase in pPB0275 (SEQ NO.: 14).

FIG. 17 shows the nucleotide sequence of codon-optimized Tp-FADdelta-5 fatty acid desaturase in pPB0276 (SEQ NO.: 15).

FIG. 18 shows the nucleotide sequence of codon-optimized Ma-FADdelta-5 fatty acid desaturase in pPB0303 (SEQ NO.: 16).

FIG. 19 shows the nucleotide sequence of codon-optimized Oblongi-FADdelta-5 fatty acid desaturase in pPB0305 (SEQ NO.: 17).

FIG. 20A-C show the nucleotide sequence of the transforming DNA contained in plasmid pPB0304 (SEQ NO.: 18).

FIG. 21A-C show the nucleotide sequence of the transforming DNA contained in plasmid pPB0306 (SEQ NO.: 19).

FIG. 22A-D show the nucleotide sequence of the transforming DNA contained in plasmid pPB0333 (SEQ NO.: 20).

FIG. 23 shows the nucleotide sequence of the Ps-FADdelta-17 contained in plasmid pPB0334 (SEQ NO.: 21).

FIGS. 24A and B show the nucleotide sequence of the ApAMT2v1-PtFADdelta-5-ApPGHUTR and ApSAD2v1-Sd-FADdelta-17-ApSAD2v1 UTR cassettes contained in plasmid pPB0338 (SEQ NO.: 22).

FIG. 25 shows the gas chromatogram showing traces amount of ARA and EPA peaks from plasmids 333, 334, and 338 transformed cells.

FIG. 26A-D show the nucleotide sequence of the transforming DNA contained in plasmid pPB0354 (SEQ NO.: 23).

### DETAILED DESCRIPTION OF THE INVENTION

### Definitions

In this disclosure, a number of terms and abbreviations are used. The following definitions are provided.

### Abbreviations

ACP—Acyl carrier protein,

FAS— Fatty acid synthase,

FATA— Fatty acyl-ACP thioesterase,

SAD— Stearoyl ACP desaturase,

FAD2— Δ12 fatty acid desaturase,

FAD3— Δ15 fatty acid desaturase,

delta-9 FAE— Δ9 fatty acid elongase,

FADdelta-8— Δ8 fatty acid desaturase,

FADdelta-5— Δ5 fatty acid desaturase,

FADdelta-17— Δ17 fatty acid desaturase,

Homomeric ACCase— Cytosolic homomeric acetyl-coenzyme A carboxylase,

GPAT— Glycerol phosphate acyltransferase,

LPAAT— Lysophosphatidic acid acyltransferase,

DGAT— Diacylglycerol acyltransferase,

PC— Phosphatidylcholine,

LPCAT— Lysophosphatidylcholine acyltransferase,

CPT or EPT— Ethanolamine choline phosphotransferase,

DAG-CPT— CDP-choline: 1,2-sn-diacylglycerolcholine phosphotransferase, or diacylglycerolcholine phosphotransferase,

PDCT— Phosphatidylcholine: 1,2-sn-diacylglycerol choline phosphotransferase, or Phosphatidylcholine: diacylglycerol choline phosphotransferase,

PDAT— Phospholipid diacylglycerol acyltransferase,

Cytb5— Cytochrome b5.

Unless otherwise defined herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner like a term “comprising.” The transitional terms/phrases (and any grammatical variations thereof) “comprising,” “comprises,” “comprise,” “consisting essentially of,” “consists essentially of,” “consisting,” and “consists of” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the value as determined by one of the ordinary skills in the art, which will depend in part on how the value is measured or determined, i.e., the limitations of the measurement system. Where values are described in the application and claims unless otherwise stated the term “about” meaning within an acceptable error range for the particular value should be assumed. In the context of compositions containing amounts of ingredients where the term “about” is used, these compositions contain the stated amount of the ingredient with a variation (error range) of 0-10% around the value (X±10%).

An “allele” refers to a version of a gene at the same place on homologous chromosomes. An allele may encode the same or similar protein.

“Exogenous gene” shall mean a nucleic acid that codes for the expression of an RNA and/or protein that has been introduced into a cell (e.g., by transformation/transfection), and is also referred to as a “transgene”. A cell comprising an exogenous gene may be referred to as a recombinant cell, into which additional exogenous gene(s) may be introduced. The exogenous gene may be from a different species (and so heterologous), or from the same species (and so homologous), relative to the cell being transformed. Thus, an exogenous gene can include a homologous gene that occupies a different location in the genome of the cell or is under different control, relative to the endogenous copy of the gene. An exogenous gene may be present in more than one copy in the cell. An exogenous gene may be maintained in a cell as an insertion into the genome (nuclear or plastid) or as an episomal molecule.

“Fatty acids” shall mean free fatty acids, fatty acid salts, or fatty acyl moieties in a glycerolipid. It will be understood that fatty acyl groups of glycerolipids can be described in terms of the carboxylic acid or anion of a carboxylic acid that is produced when the triglyceride is hydrolyzed or saponified.

“Fixed carbon source” is a molecule(s) containing carbon, typically an organic molecule that is present at ambient temperature and pressure in solid or liquid form in a culture media that can be utilized by a microorganism cultured therein. Accordingly, carbon dioxide is not a fixed carbon source.

“Microalgae” are eukaryotic microbial organisms that contain a chloroplast or other plastid, and optionally that can perform photosynthesis, or a prokaryotic microbial organism capable of performing photosynthesis. Microalgae include obligate photoautotrophs, which cannot metabolize a fixed carbon source as energy, as well as heterotrophs, which can live solely off a fixed carbon source. Microalgae include unicellular organisms that separate from sister cells shortly after cell division, such as Chlamydomonas, as well as microbes such as, for example, volvox, which is a simple multicellular photosynthetic microbe of two distinct cell types. Microalgae include cells such as Chlorella, Auxenochlorella, Dunaliella, and Prototheca. Microalgae also include other microbial photosynthetic organisms that exhibit cell-cell adhesion, such as Agmenellum, Anabaena, and Pyrobotrys. Microalgae also include obligate heterotrophic microorganisms that have lost the ability to perform photosynthesis.

In connection with a recombinant cell, the term “knockdown” refers to a gene that has been partially suppressed (e.g., by about 1-95%) in terms of the production or activity of a protein encoded by the gene.

Also, in connection with a recombinant cell, the term “knockout” refers to a gene that has been completely or nearly completely (e.g., >95%) suppressed in terms of the production or activity of a protein encoded by the gene. Knockouts can be prepared by ablating the gene by homologous recombination of a nucleic acid sequence into a coding sequence, gene deletion, mutation, or other methods. When homologous recombination is performed, the nucleic acid that is inserted (“knocked-in”) can be a sequence that encodes an exogenous gene of interest or a sequence that does not encode for a gene of interest.

An “oleaginous” cell is a cell capable of producing at least 20% lipid by dry cell weight, naturally or through recombinant or classical strain improvement. An “oleaginous microbe” or “oleaginous microorganism” is a microbe, including a microalga that is oleaginous (especially eukaryotic microalgae that store lipids). An oleaginous cell also encompasses a cell that has had some or all its lipid or other content removed, and both live and dead cells. In connection with a functional oil, a “profile” is the distribution of species or triglycerides or fatty acyl groups within the oil. A “fatty acid profile” is the distribution of fatty acyl groups in the triglycerides of the oil without reference to the attachment to a glycerol backbone. Fatty acid profiles are typically determined by conversion to a fatty acid methyl ester (FAME), followed by gas chromatography (GC) analysis with flame ionization detection (FID). The fatty acid profile can be expressed as one or more percent of fatty acid in the total fatty acid signal determined from the area under the curve for that fatty acid.

“Recombinant” is a cell, nucleic acid, protein, or vector that has been modified due to the introduction of an exogenous nucleic acid or the alteration of a native nucleic acid. Thus, e.g., recombinant cells can express genes that are not found within the native (non-recombinant) form of the cell or express native genes differently than those genes expressed by a non-recombinant cell. Recombinant cells can, without limitation, include recombinant nucleic acids that encode for a gene product or for suppression elements such as mutations, knockouts, antisense, interfering RNA (RNAi), or dsRNA that reduce the levels of the active gene product in a cell. A “recombinant nucleic acid” is a nucleic acid originally formed in vitro, in general, by the manipulation of nucleic acid, e.g., using polymerases, ligases, exonucleases, and endonucleases, using chemical synthesis, or otherwise is in a form not normally found in nature. Recombinant nucleic acids may be produced, for example, to place two or more nucleic acids in operable linkage. Thus, an isolated nucleic acid or an expression vector formed in vitro by ligating DNA molecules that are not normally joined in nature, are both considered recombinant for the purposes of this invention. Once a recombinant nucleic acid is made and introduced into a host cell or organism, it may replicate using the in vivo cellular machinery of the host cell; however, such nucleic acids, once produced recombinantly, although subsequently replicated intracellularly, are still considered recombinant for purposes of this invention. Similarly, a “recombinant protein” is a protein made using recombinant techniques, i.e., through the expression of a recombinant nucleic acid.

“Cultivated”, and variants thereof such as “cultured” and “fermented”, refer to the intentional fostering of growth (increases in cell size, cellular contents, and/or cellular activity) and/or propagation (increases in cell numbers via mitosis) of one or more cells by use of selected and/or controlled conditions. The combination of both growth and propagation may be termed proliferation. Examples of selected and/or controlled conditions include the use of a defined medium (with known characteristics such as pH, ionic strength, and carbon source), specified temperature, oxygen tension, carbon dioxide levels, and growth in a bioreactor. Cultivate does not refer to the growth or propagation of microorganisms in nature or otherwise without human intervention; for example, the natural growth of an organism that ultimately becomes fossilized to produce geological crude oil is not cultivated.

“Desaturase” are enzymes in the lipid synthesis pathway responsible for the introduction of double bonds (unsaturation) into the fatty acid chains of fatty acid or triacylglyceride molecules. Examples include but are not limited to fatty acid desaturase (FAD), also known as fatty acyl desaturase.

In the present disclosure, ranges are stated in shorthand, to avoid having to set out at length and describe each value within the range. Any appropriate value within the range can be selected, where appropriate, as the upper value, lower value, or the terminus of the range.

For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. When ranges are used herein, combinations and sub-combinations of ranges (e.g., subranges within the disclosed range), specific embodiments therein are intended to be explicitly included.

Hereinafter, the present invention will be explained in detail.

As an aspect for achieving the object of the present invention, the present invention provides an Auxenochlorella protothecoides mutant for producing high-value essential LCPUFA oils.

Among the oleaginous microalgae, Auxenochlorella genus, more preferably, Auxenochlorella protothecoides was selected as the preferred microalgal host for the purposes herein. In a specific embodiment, the Auxenochlorella protothecoides may be Auxenochlorella protothecoides PB5 disclosed in U.S. application Ser. No. 17/519,854.

Auxenochlorella protothecoides is a superior system for generating engineered microalgae strains due to its intrinsic ability to accumulate copious amounts of oil as triglycerides most of which is accumulated as C18:1n-9 (oleic acid) that can be efficiently channeled into phospholipids to produce LCPUFAs as presented in the examples herein, and its ease of transformation and facile homologous recombination into the nuclear genome that does not require riboprotein-mediated gene editing, facilitating gene targeting, and express heterologous genes well. It is a robust organism that grows rapidly and performs well under industrial fermentation conditions (e.g., in high-pressured fermenters). Also, PB5 has a higher intrinsic capacity than non-photosynthetic heterotrophic platforms for the production of carotenoids and other terpenoids due to the high flux through these biosynthetic pathways during photosynthetic growth. There is also a benefit to using a green alga as a host for the expression of plant proteins, as the compartment of cellular compartments and cofactors is similar.

As an embodiment, the Auxenochlorella protothecoides mutant may be a mutant of A. protothecoides, PB5.

Wild type A. protothecoides PB5 was obtained from the University of Texas Culture Collection of Algae (UTEX catalog number 250), and it is available to the public to purchase via the webpage www.utex.org.

The mutants of the present invention are more industrially useful in that they may provide oils having fatty acids content of a profile different from that produced in wild-type Auxenochlorella protothecoides.

The Auxenochlorella protothecoides mutant of the present invention may be prepared using general mutation treatment methods.

In the present invention, “mutation” refers to a change in a nucleotide sequence due to the insertion, deletion, or substitution of a base into the original nucleotide sequence. As a means of mutation, the number of inserted bases may be different depending on the mutation and thus is not limited thereto. “Deletion mutation” means a mutation in which a base is removed from the original nucleotide sequence, and “substitution mutation” means that an original nucleotide is changed to another base without changing the number in the original nucleotide sequence.

In one embodiment of the present invention, the microalgal mutant is modified to alter the expression level of a combination of at least one gene encoding elongase and at least one gene encoding desaturases.

In a specific embodiment, the Auxenochlorella protothecoides mutant may be recombinant Auxenochlorella protothecoides for the production of long-chain polyunsaturated fatty acid comprising:

a combination of at least one gene encoding elongase and at least one gene encoding desaturases.

Preferably, the elongase is delta-9 elongase (delta-9FAE).

Preferably, the desaturase is at least one gene selected from a group consisting of:

a) gene encoding delta-8 desaturase (FADdelta-8);

b) gene encoding delta-5 desaturase (FADdelta-5); and

c) gene encoding delta-17 desaturases (FADdelta-17).

In the specific embodiment, the delta-9 elongase may be delta-9 elongase from Euglena gracilis, Isochrysis galbana, or Pavlova pinguis. The heterologous delta-9FAE and FADdelta-8 in the recombinant Auxenochlorella protothecoides convert the available C18:2n-6 to EDA.

In the specific embodiment, the delta-8 desaturases may be delta-8 desaturases from E. gracilis, Perkinus marinus, I. galbana, P olseni, Mortierella sp. NVP85, Mortierella alpina, Diacronema lutheri, Pavlovales sp. CCMP2436, Pavlova salina, or Capsaspora owczarzaki. The heterologous FADdelta-8 in the recombinant Auxenochlorella protothecoides convert the EDA to DGLA.

In the specific embodiment, the delta-5 desaturases may be delta-5 desaturases from Phaeodactylum tricornutum, Dictyostelium discoideum, M. alpina, C. elegans, Oblongichytrium sp. SEK 347, Euglena gracilis, Parietochloris incisa, or Thalassiosira pseudonana CCMP1335. The heterologous FADdelta-5 desaturase enzymes in the recombinant Auxenochlorella protothecoides convert DGLA to ARA.

In the specific embodiment, the delta-17 desaturases may be delta-17 desaturases from Pythium aphanidermatum, Phytophthora sojae, Phytophthora ramorum, or Saprolegnia diclina. The heterologous FADdelta-17 desaturase enzymes in the recombinant Auxenochlorella protothecoides convert ARA to EPA, respectively.

Preferably, the recombinant Auxenochlorella protothecoides for production of long-chain polyunsaturated fatty acid further comprising at least one gene selected from a group consisting of:
a) gene encoding lysophosphatidylcholine acyltransferase (LPCAT);
b) gene encoding lysophosphatidic acid acyltransferase (LPAAT);
c) gene encoding cytochrome b5; and
d) gene encoding choline phosphotransferase (CPT); and functional equivalents thereof.

The above genes promote the production of long-chain polyunsaturated fatty acid in the Auxenochlorella protothecoides.

LCPUFA biosynthesis in the Auxenochlorella protothecoides mutant is increased by the overexpression of heterologous LPCAT in the recombinant Auxenochlorella protothecoides mutant to effectively channel C18:1n-9 through phospholipids, compared to wild-type microalga.

In a specific embodiment, the present invention provides a recombinant Auxenochlorella protothecoides to produce EPA, comprising one or more genes encoding lysophosphatidylcholine acyltransferase (LPCAT), delta-9 elongase (delta-9FAE), delta-8 desaturase (FADdelta-8), delta-5 desaturase (FADdelta-5), delta-17 desaturases (FADdelta-17), lysophosphatidic acid acyltransferase (LPAAT), cytochrome b5 (Cytb5), choline phosphotransferase (CPT) and functional equivalents thereof.

In a specific embodiment, the recombinant Auxenochlorella protothecoides to produce LCPUFA of the present invention may comprise one or more genes encoding various protein sequences such as:

lysophosphatidylcholine acyltransferase (LPCAT) from Arabidopsis thaliana, B. rapa or B. napus;

phosphatidylcholine: 1,2-sn-diacylglycerol choline phosphotransferase (PDCT) from A. thaliana;

specific choline/ethanolamine phosphotransferase (CPT/EPT);

diacylglycerol cholinephosphotransferase (DAG-CPT);

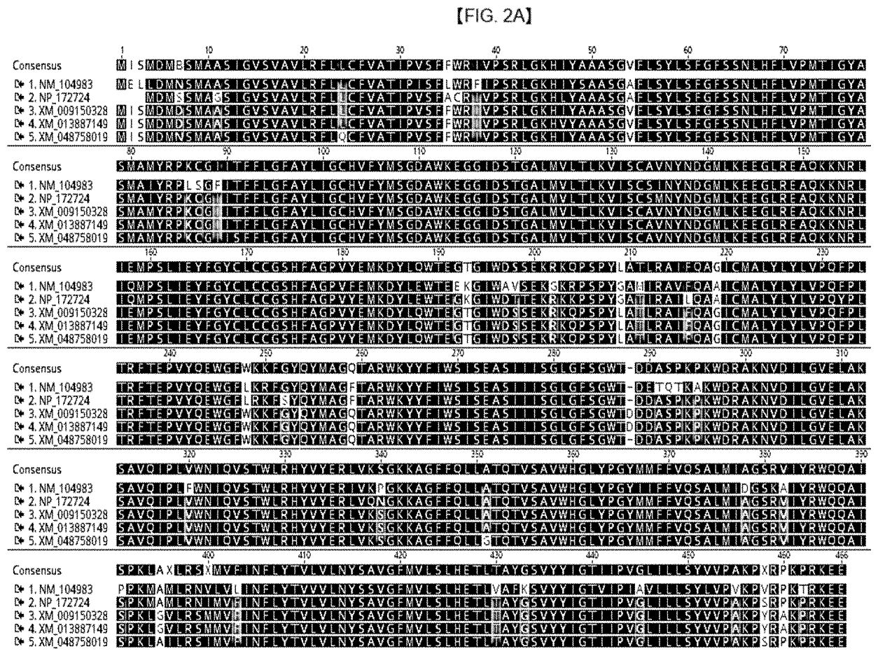

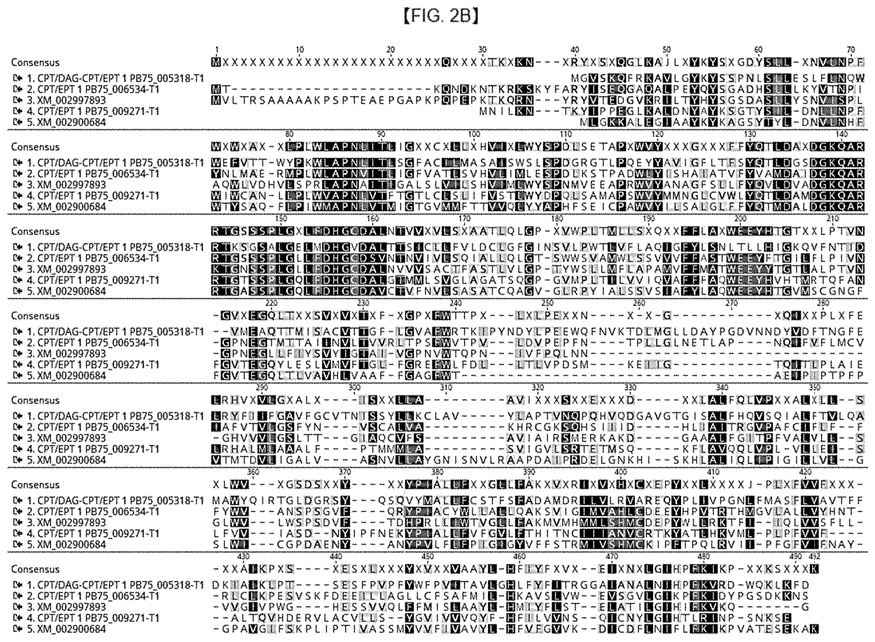

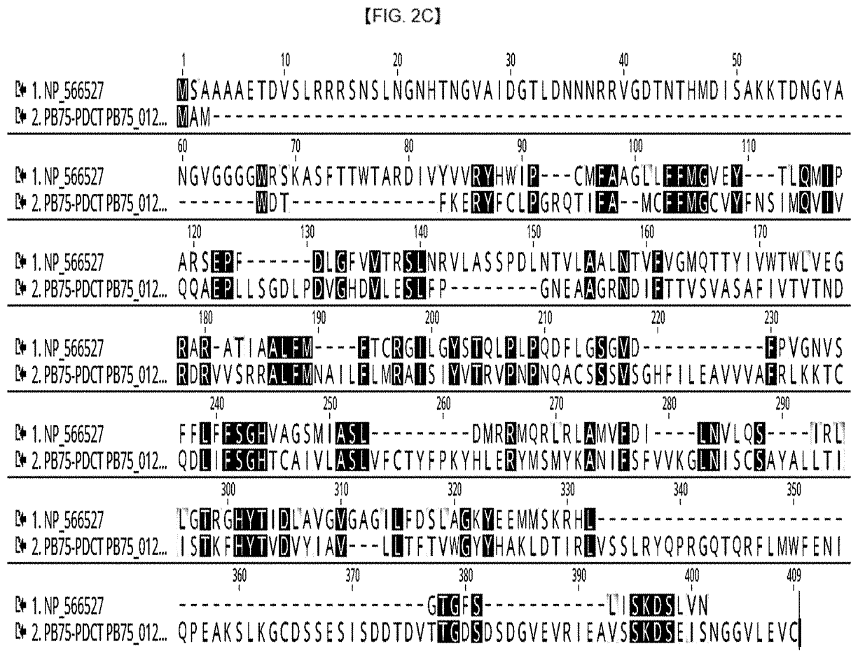

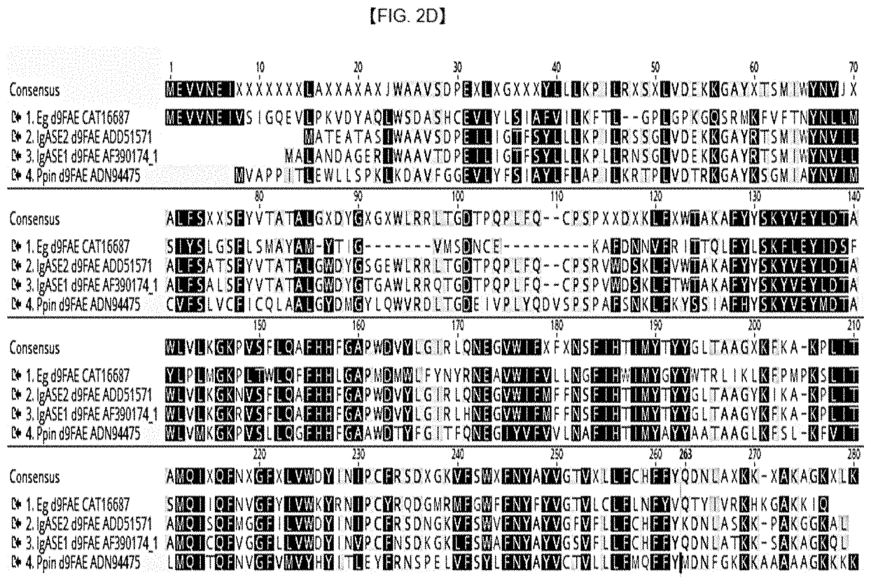

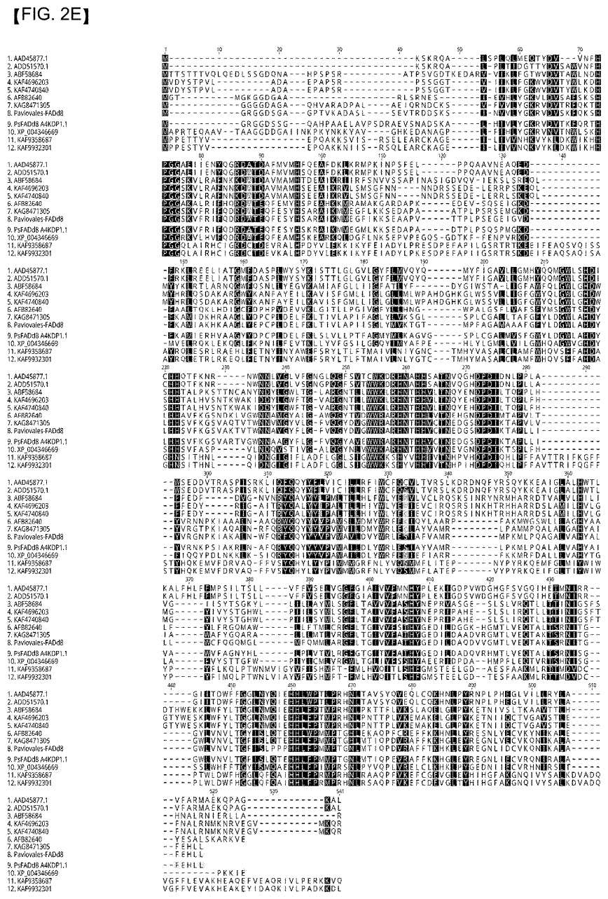

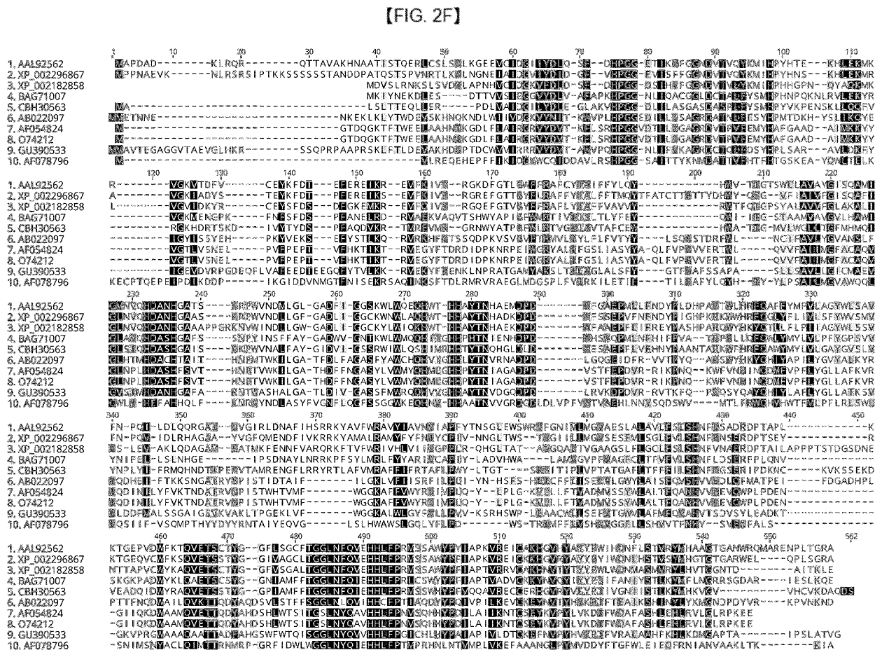

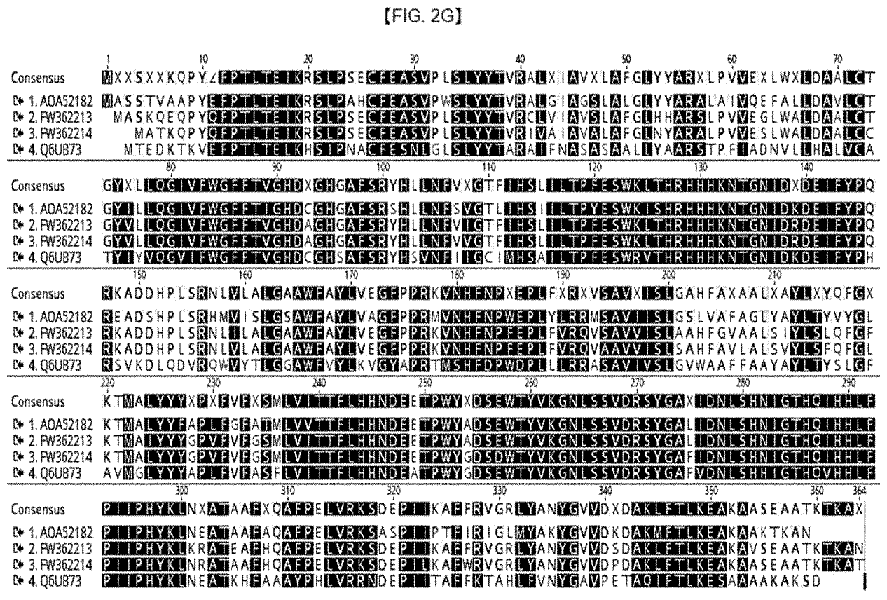

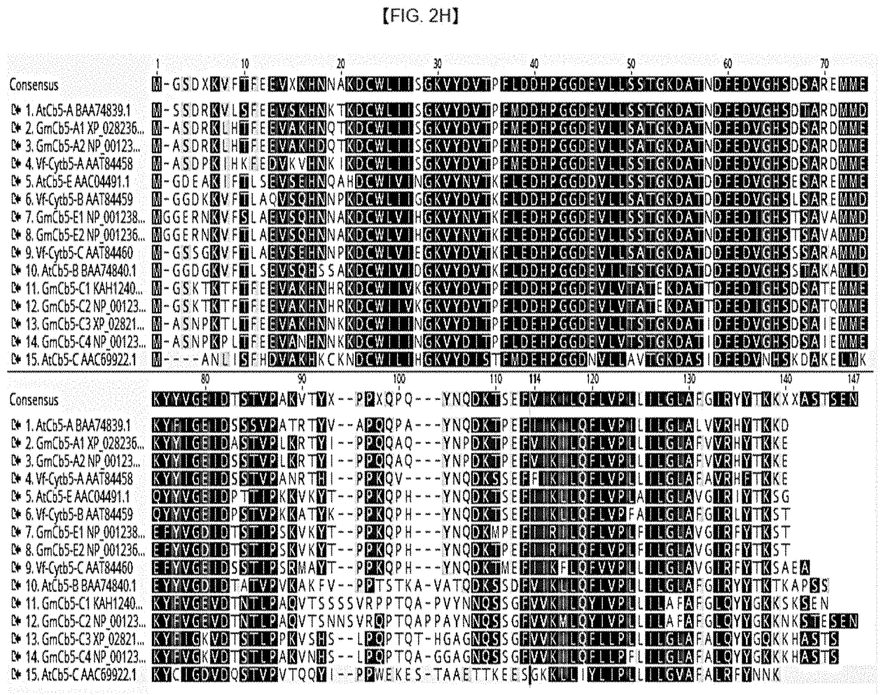

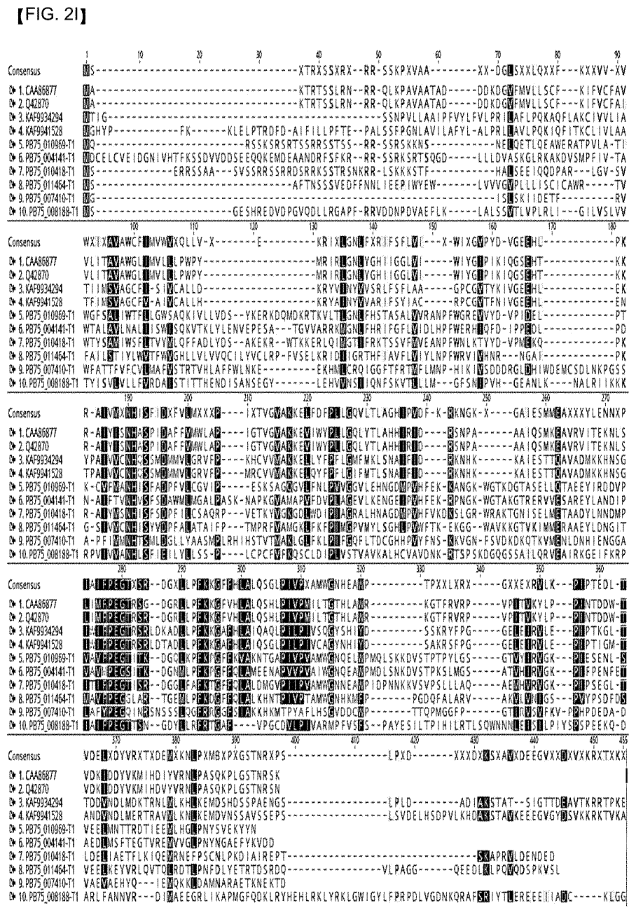

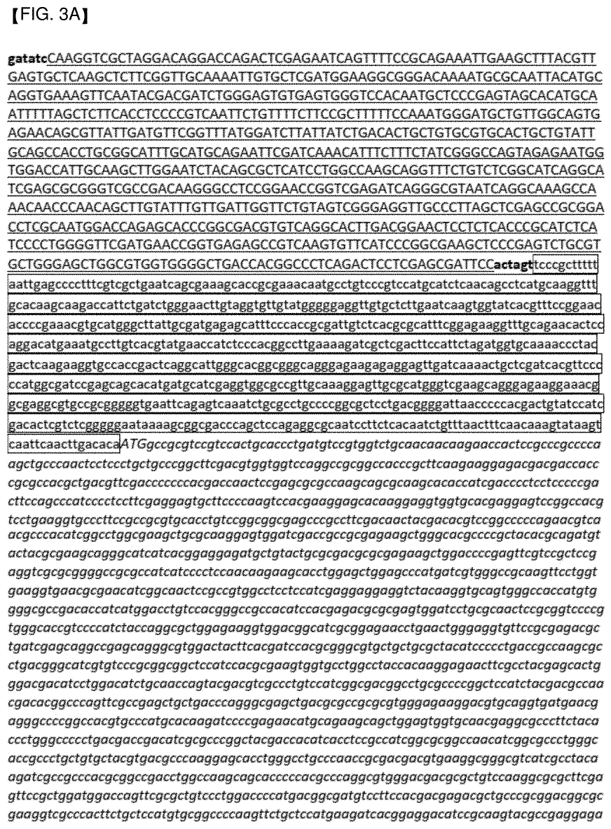

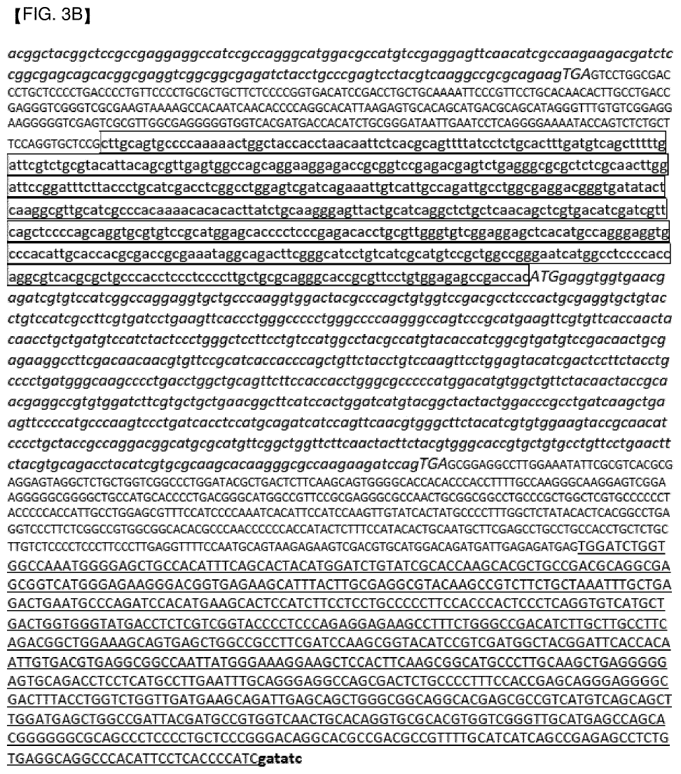

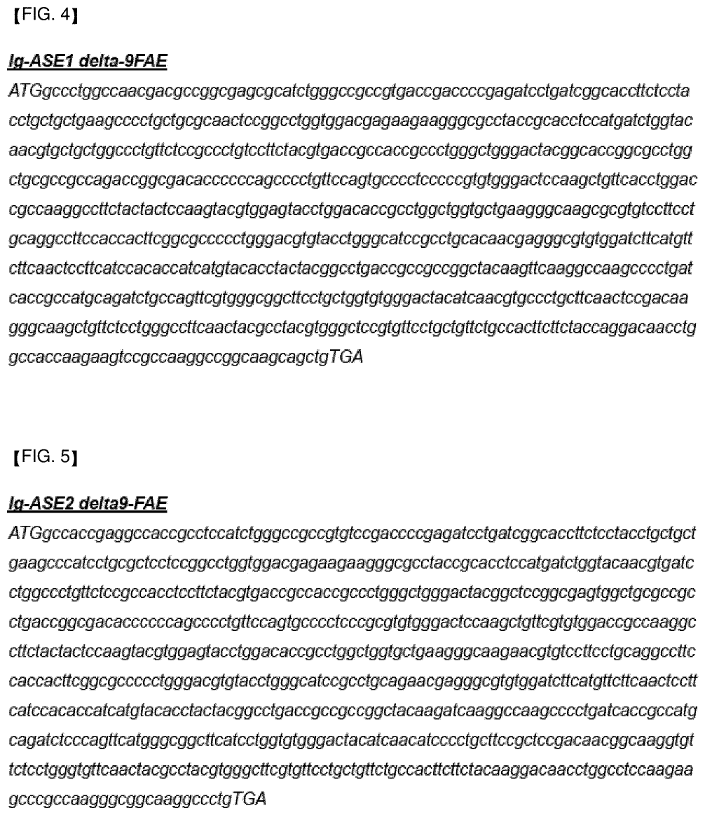

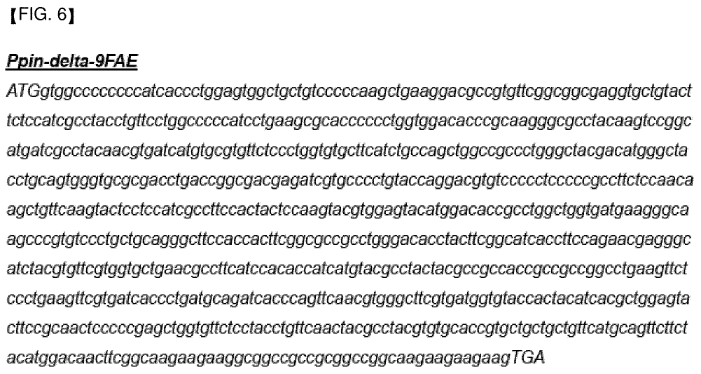

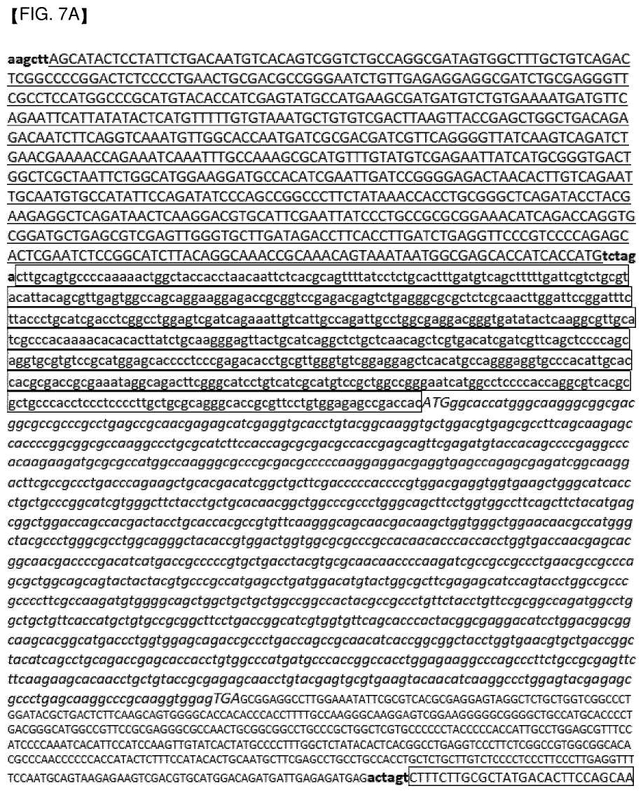

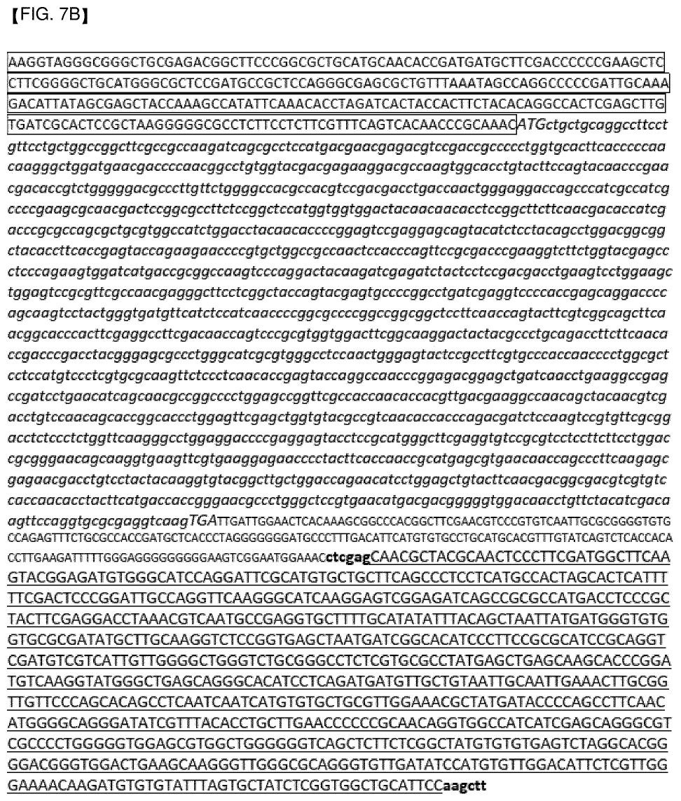

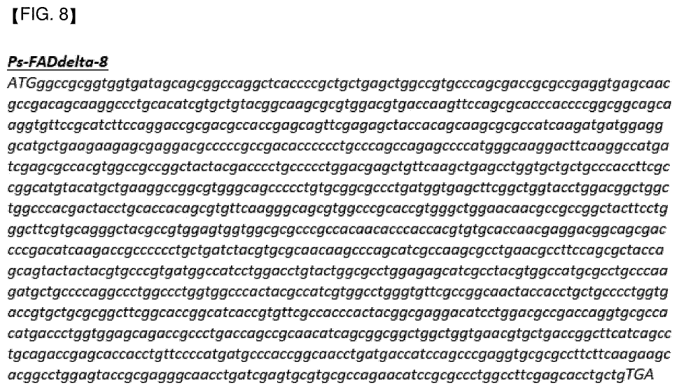

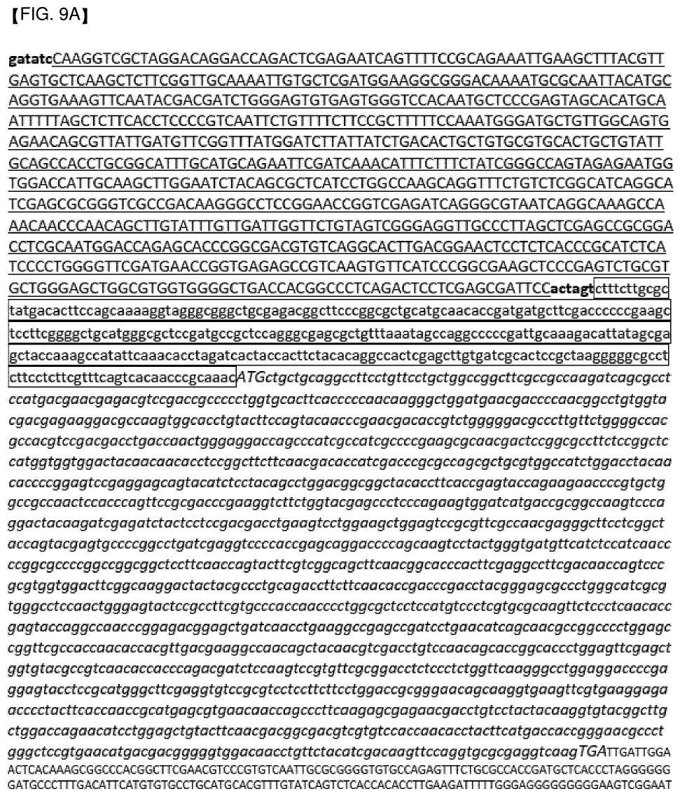

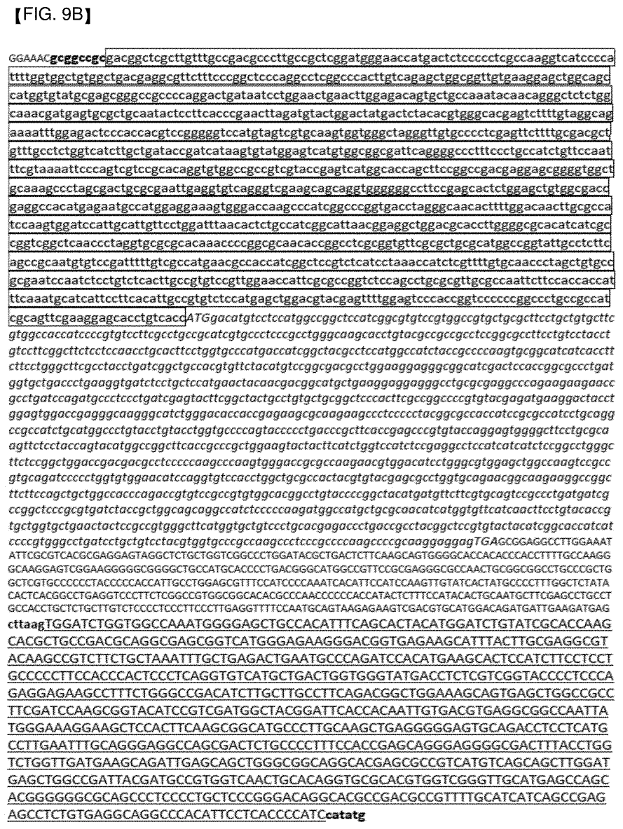

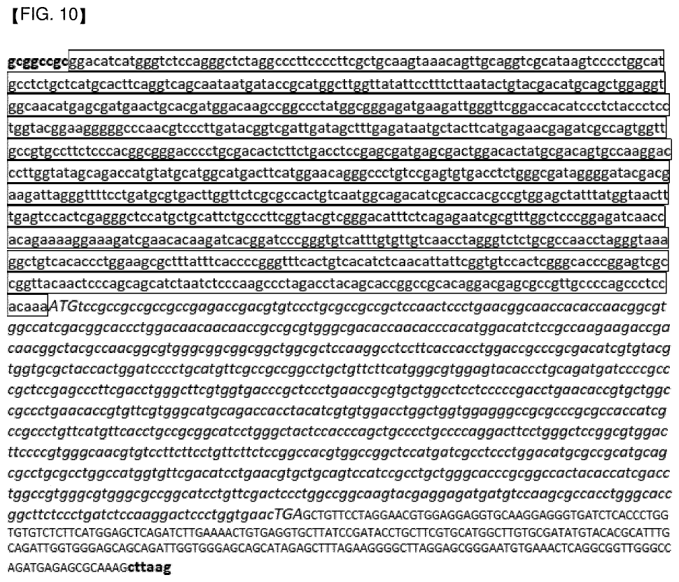

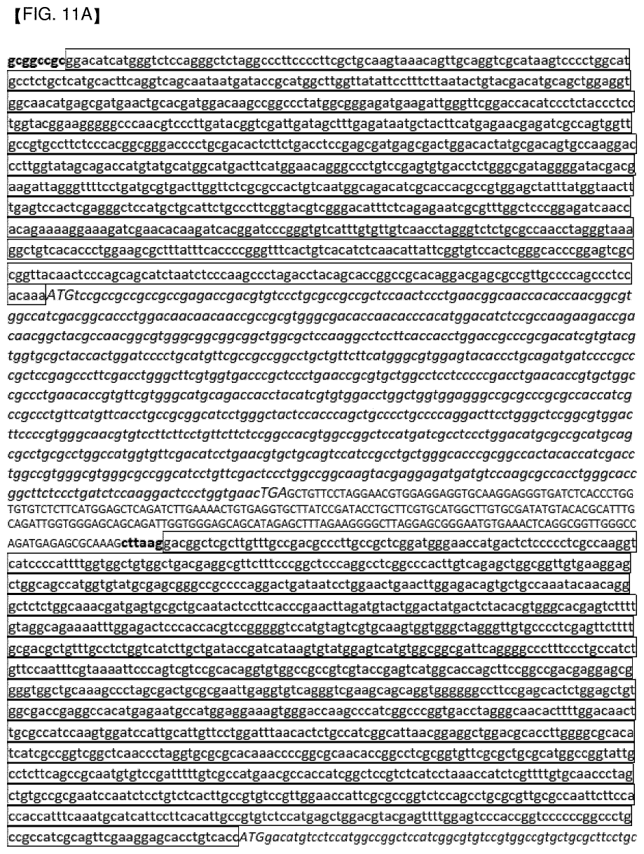

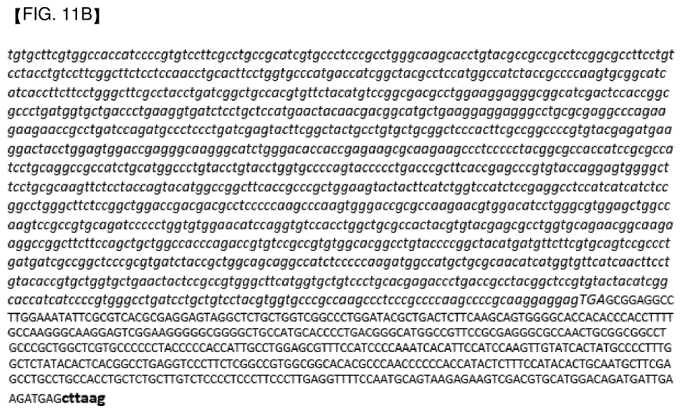

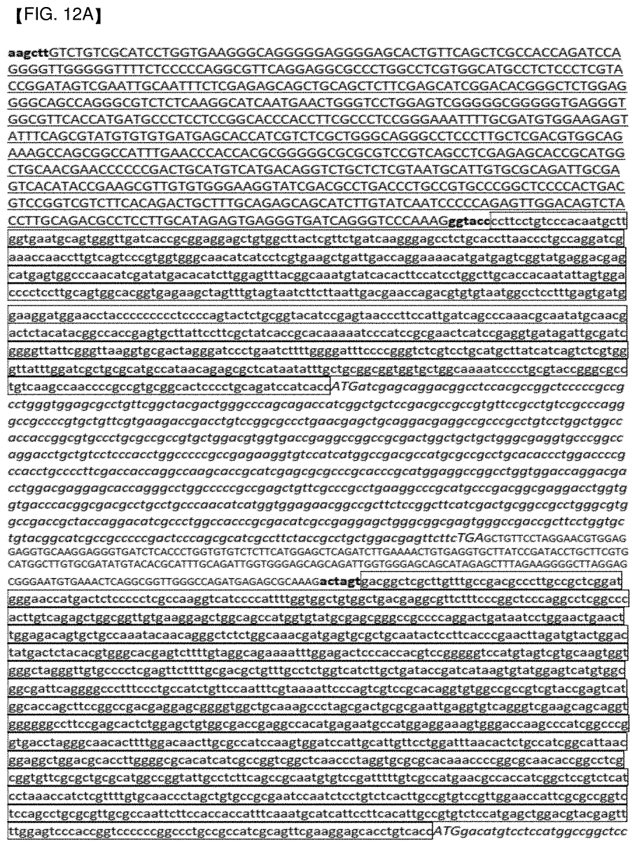

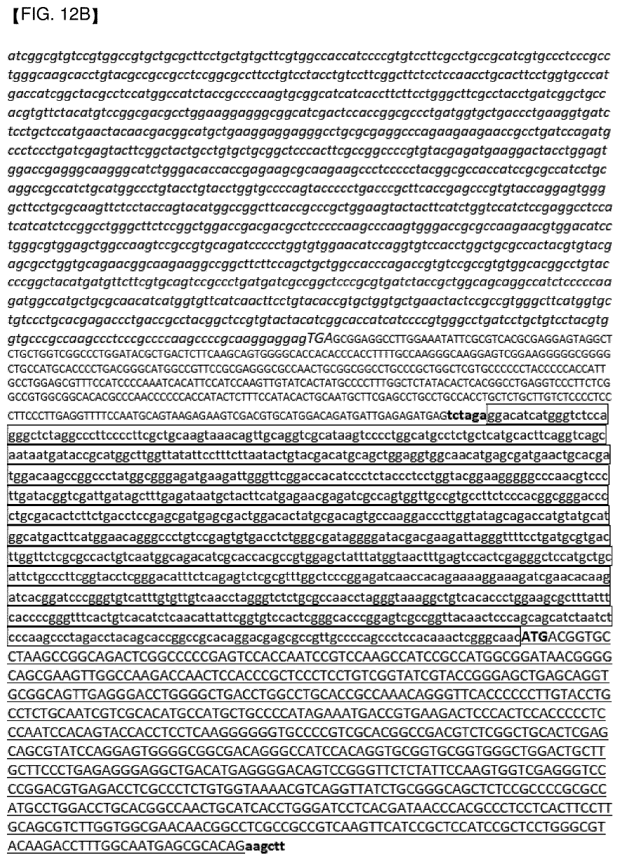

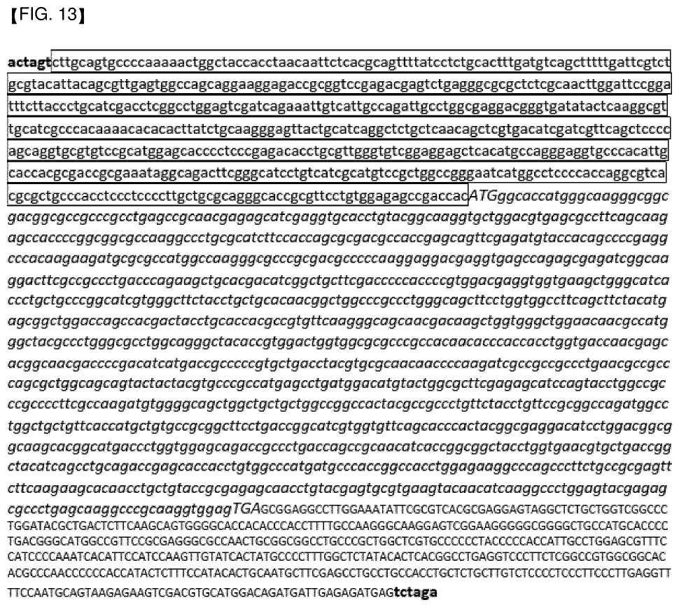

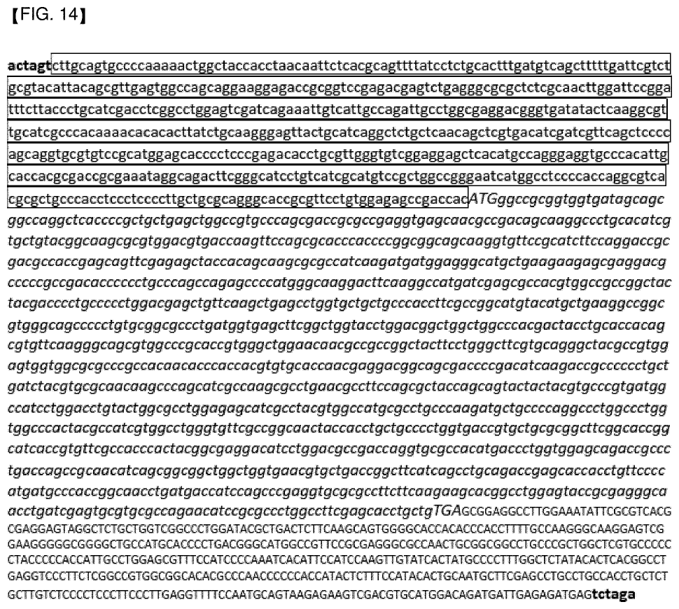

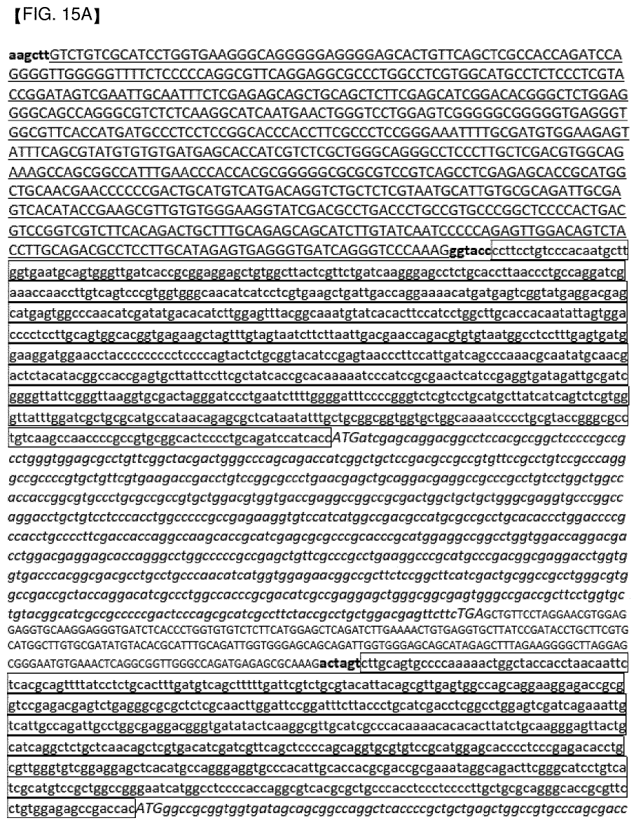

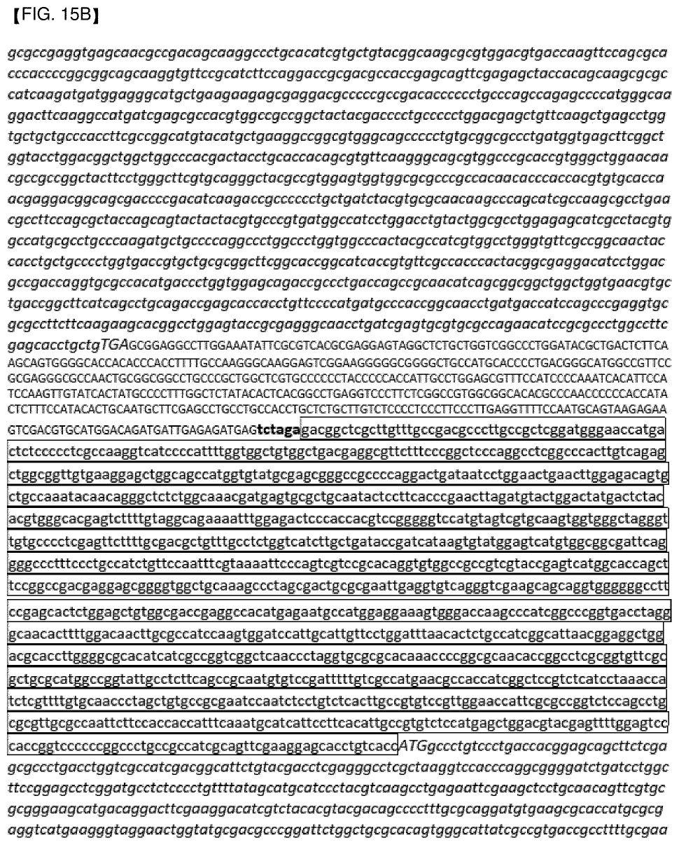

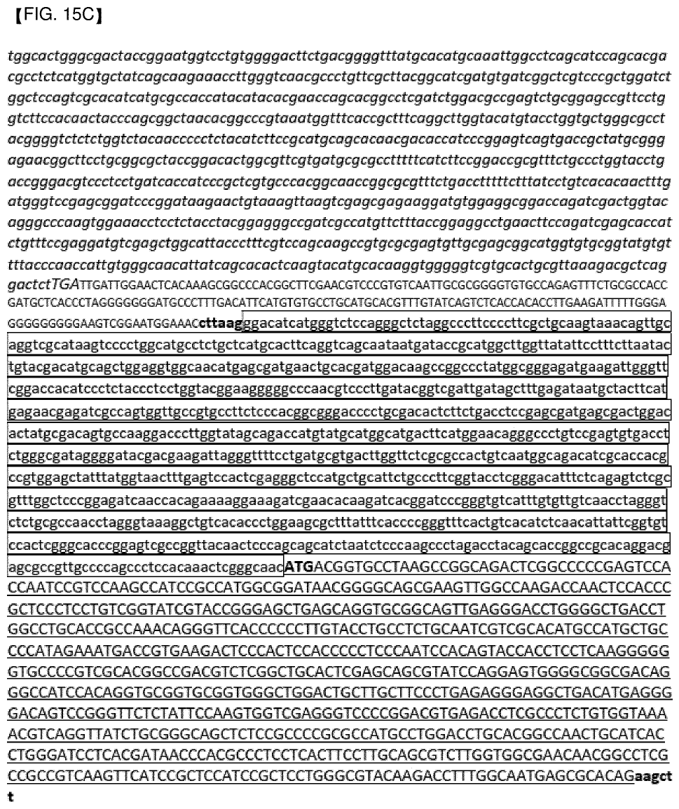

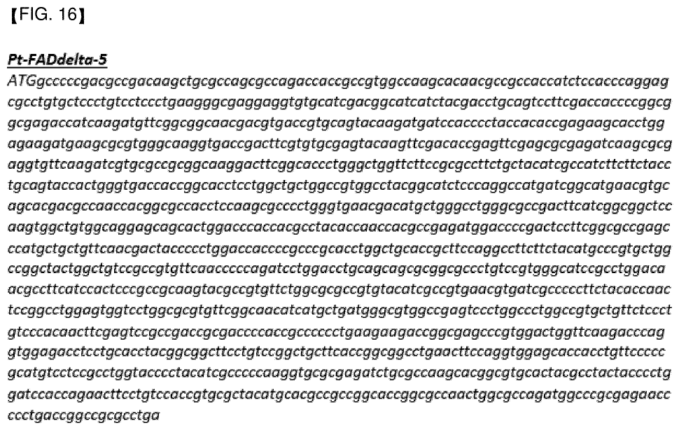

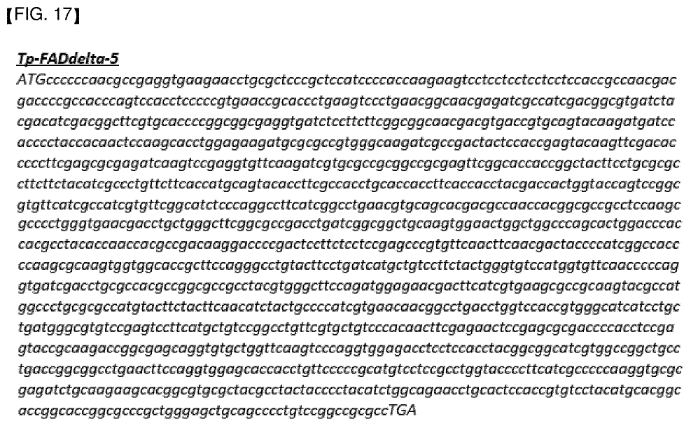

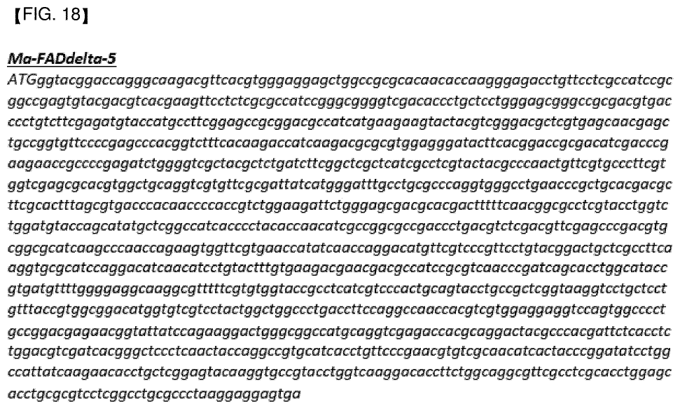

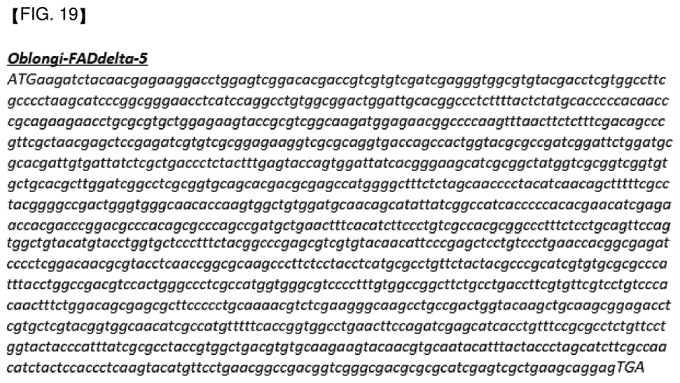

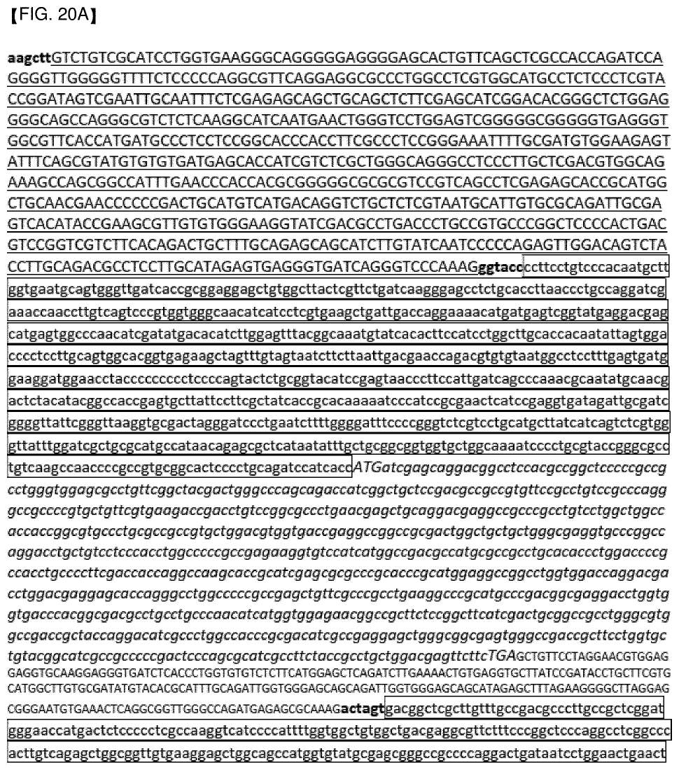

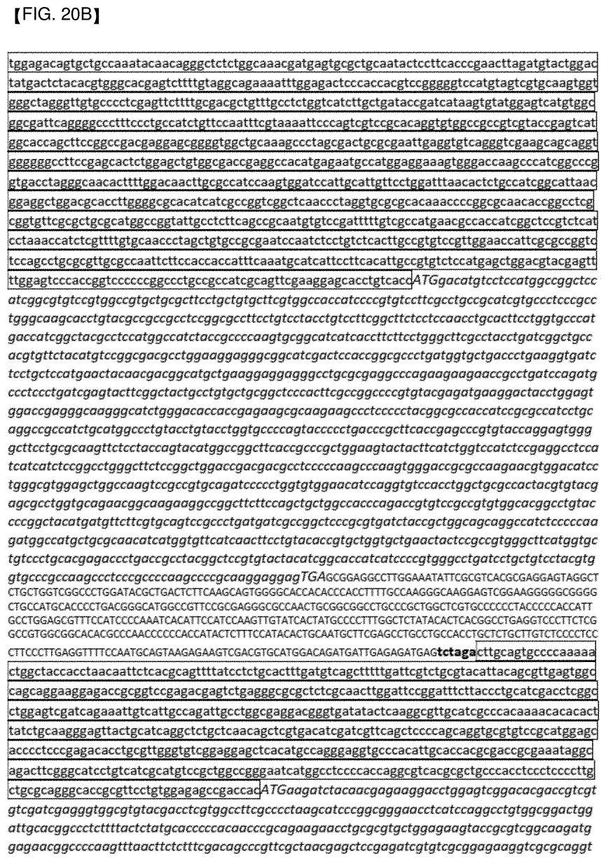

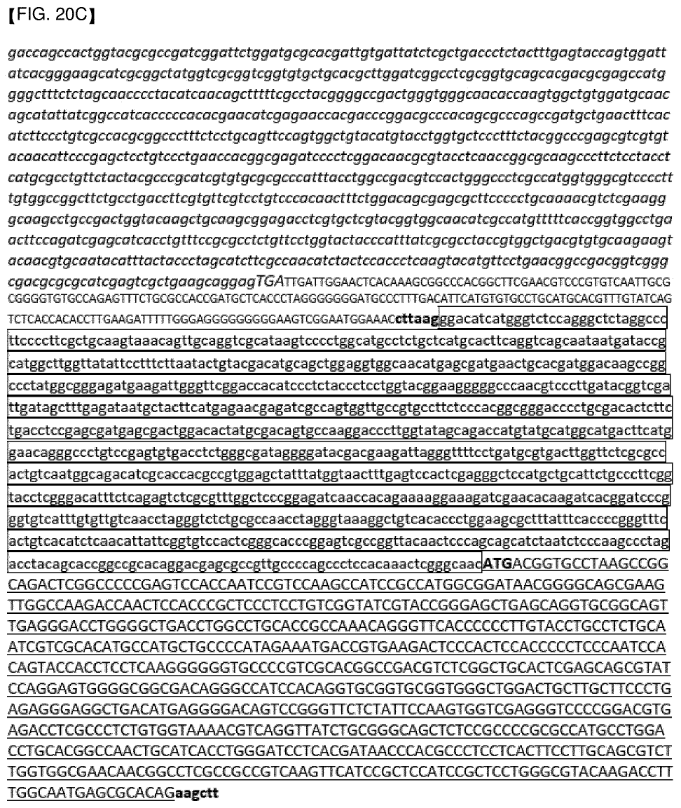

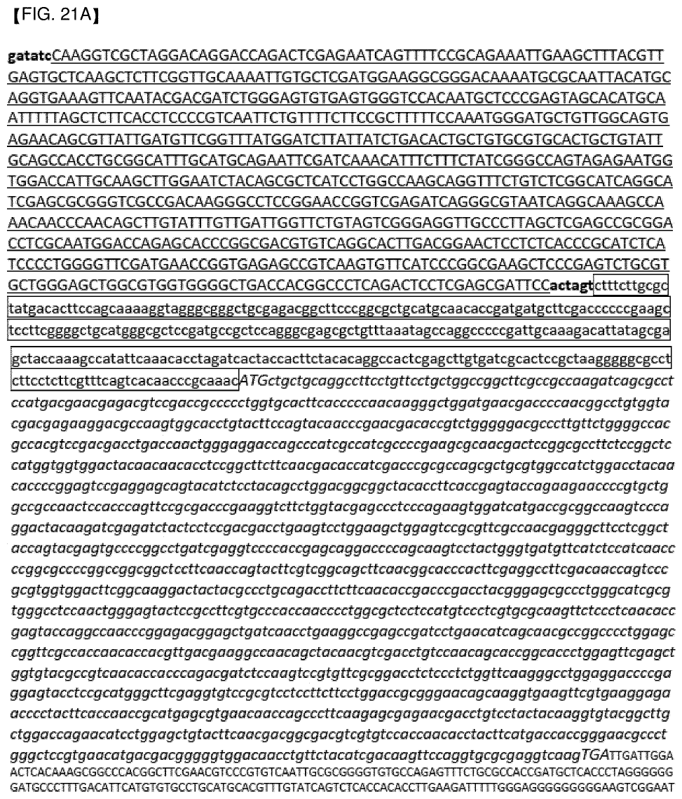

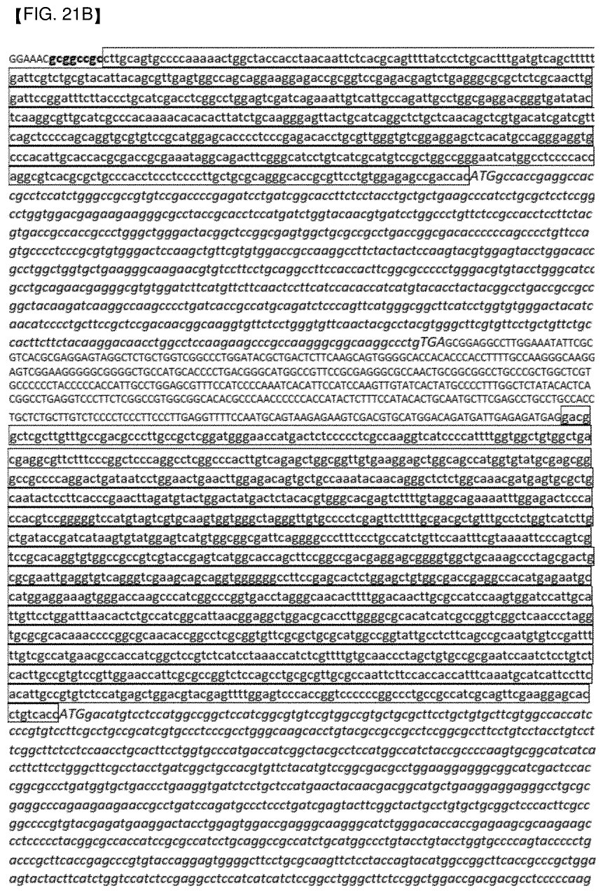

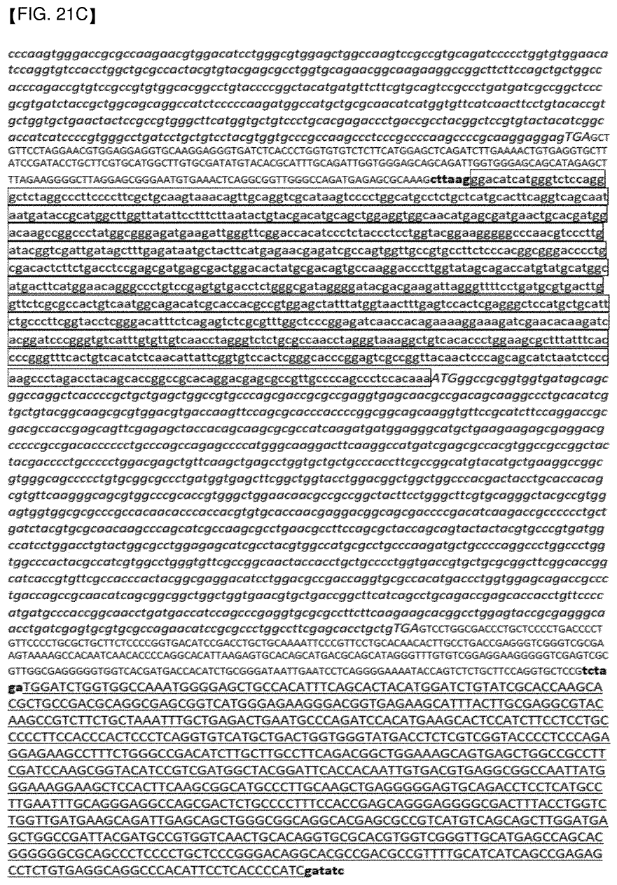

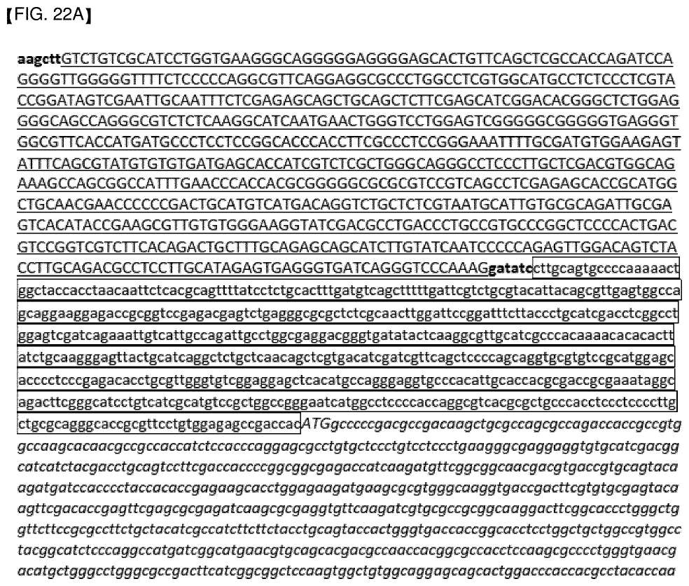

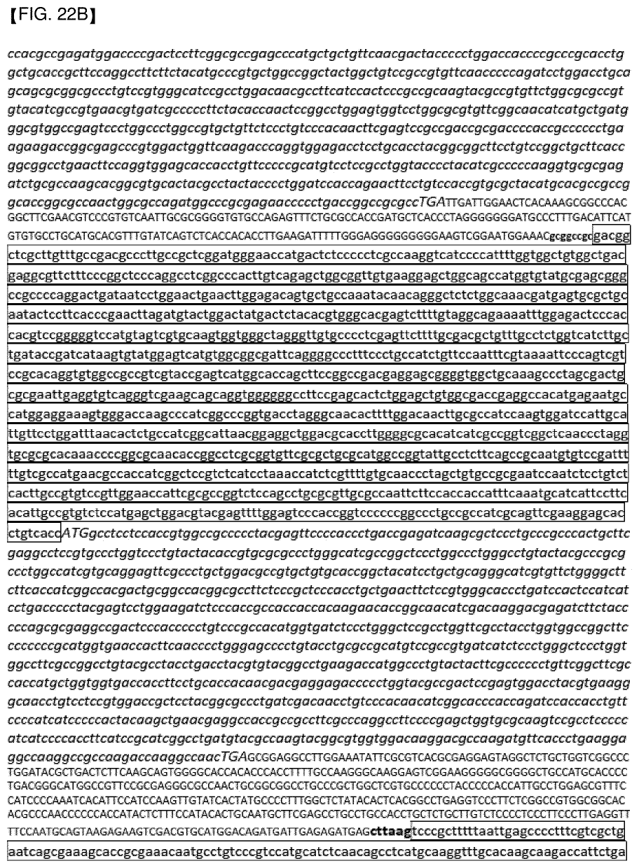

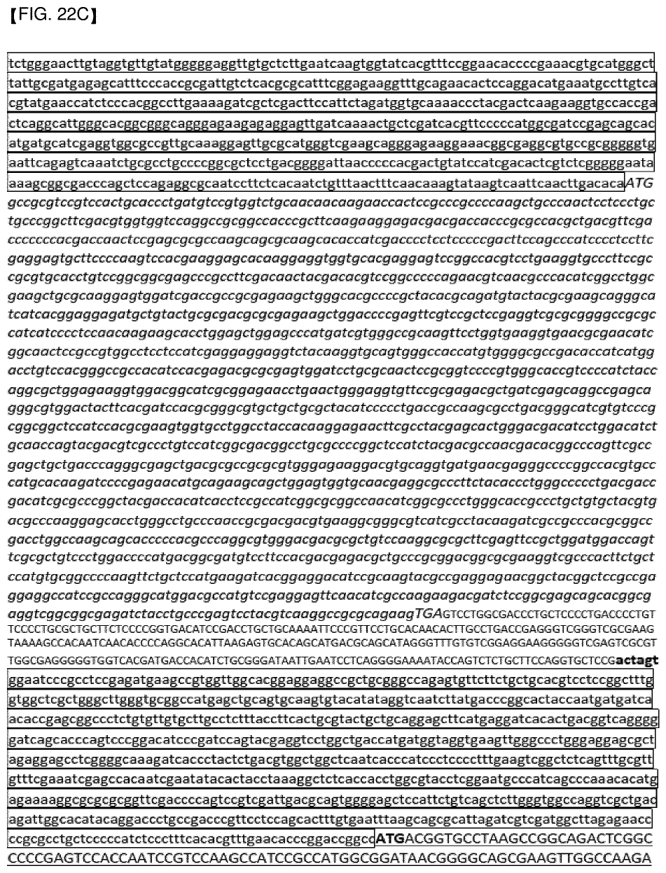

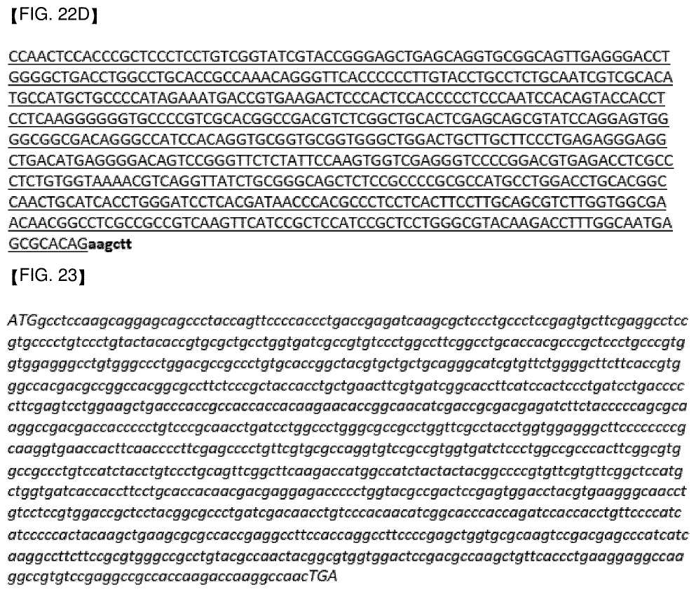

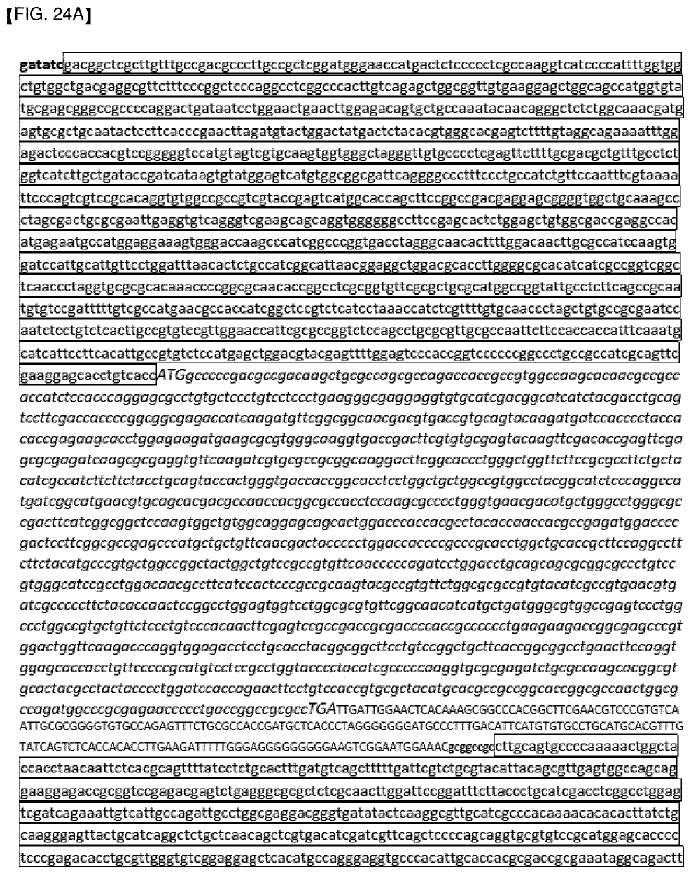

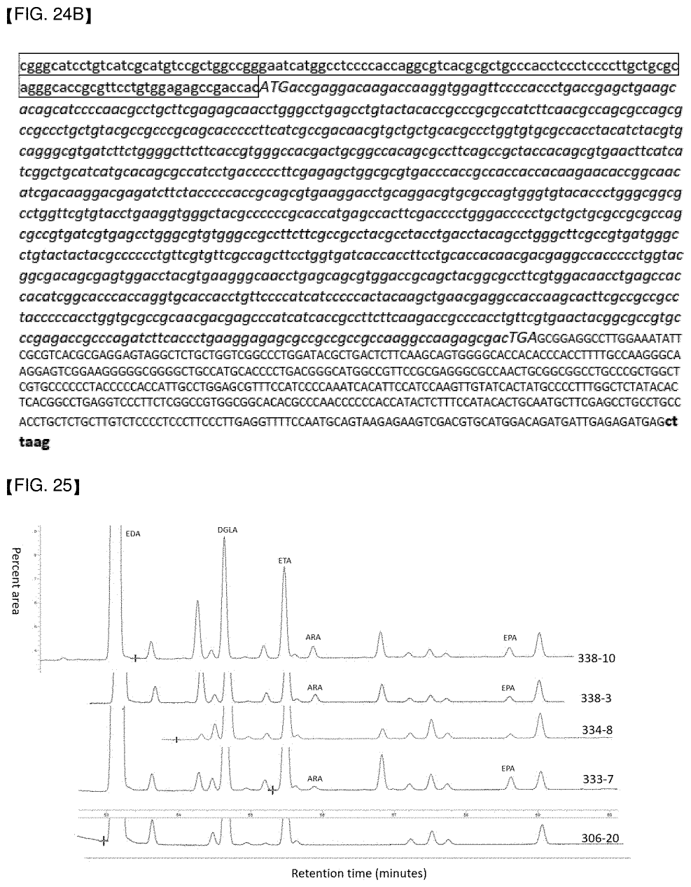

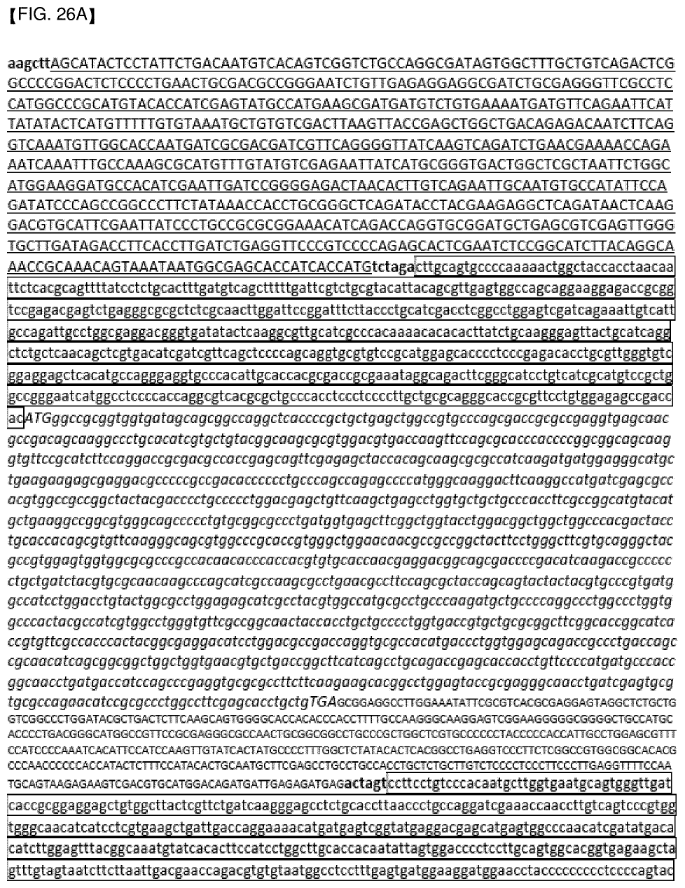

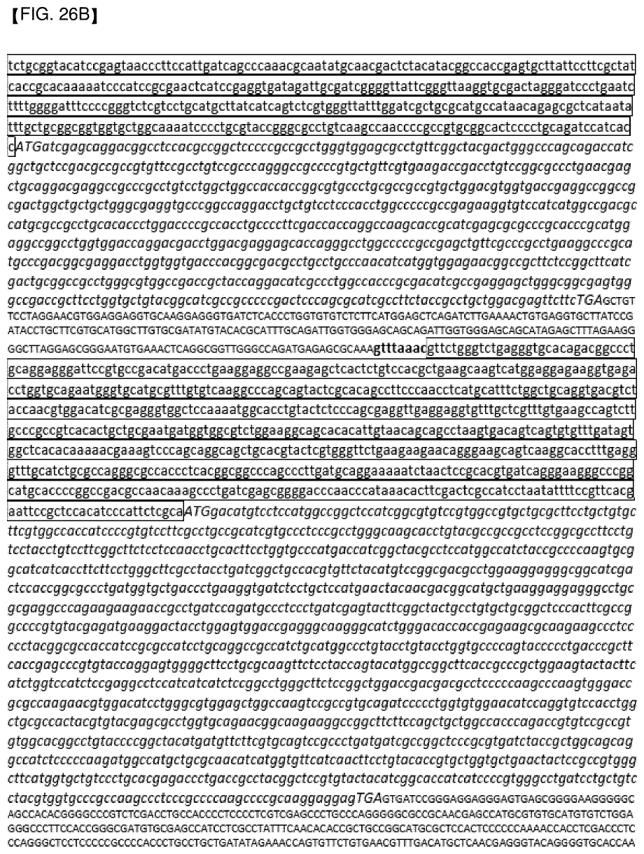

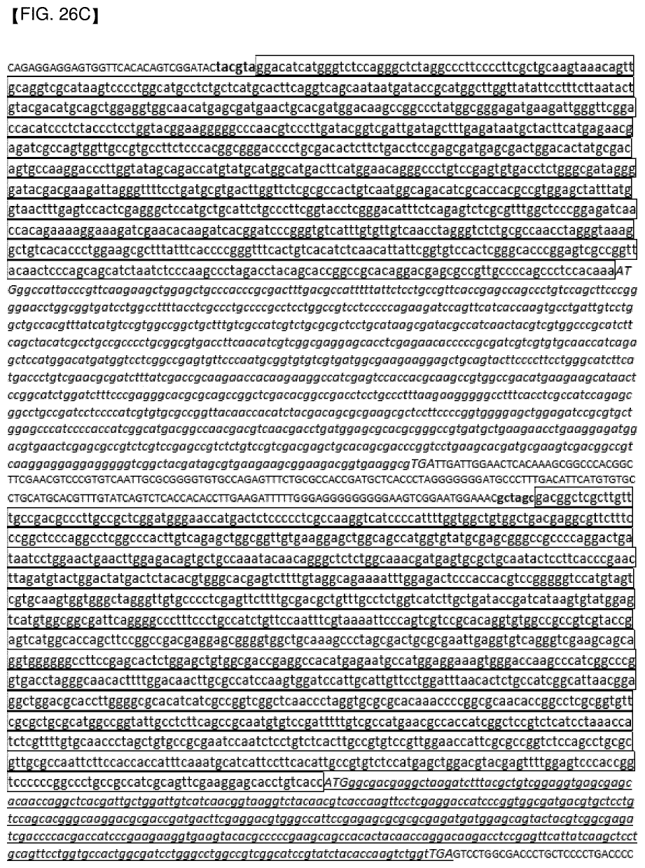

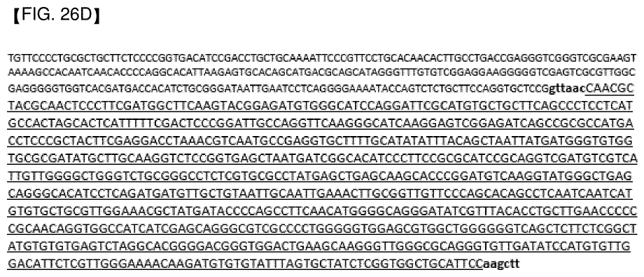

phosphatidyl-choline: 1,2-sn-diacylglycerol cholinephosphotransferase (PDCT)-like enzymes from a Phycoil proprietary strain PB75 (Oblongichytrium sp., as shown in FIG. 2.); delta-9 elongase from Euglena gracilis, Isochrysis galbana or Pavlova pinguis; delta-8 desaturases from E. gracilis, Perkinus marinus, I. galbana, P. olseni, Mortierella sp. NVP85, Mortierella alpina, Diacronema lutheri, Pavlovales sp. CCMP2436, Pavlova salina, or Capsaspora owczarzaki; or

delta-5 desaturases from Phaeodactylum tricornutum, Dictyostelium discoideum, M. alpina, C. elegans, Oblongichytrium sp. SEK 347, Euglena gracilis, Parietochloris incisa, or Thalassiosira pseudonana CCMP1335, and

delta-17 desaturases from Pythium aphanidermatum, Phytophthora sojae, Phytophthora ramorum, or Saprolegnia diclina.

In a specific embodiment of the present invention, the microalgal mutant comprises recombinant nucleic acids encoding heterologous gene expressing one or more selected from a group consisting of Arabidopsis thaliana lysophosphatidylcholine acyltransferase (At-LPCAT1; Accession No: NP_172724) and phosphatidyl-choline: diacyl-glycerol-choline phosphotransferase (At-PDCT, Accession No: NP_566527), delta-9 elongase from Euglena gracilis (Eg-delta-9 FAE, Accession number: CAT16687), Isochrysis galbana (Ig-delta-9 FAE, Accession No's: AF390174_1 and ADD51571) and Pavlova pinguis (Ppin-delta-9 FAE; Accession No: ADN94475), delta-8 desaturase from I. galbana (Ig-FADdelta-8; Accession No: AFB82640) and Pavlova salina (Ps-FADdelta-8; Accession No: Δ4KDP1.1), delta-5 desaturase from Phaeodactylum tricornutum (Pt-FAD delta-5; Accession No: AAL92562), and delta-17 desaturases from Pythium aphanidermatum (Pa-FADdelta-17; Accession No: AOA52182), Phytophthora sojae (Ps-FADdelta-17; Accession No: FW362213) and Saprolegnia diclina (Sd-FADdelta-17; Accession No: Q6UB73).

The inventors of the present invention aim to produce high-value essential LCPUFA oils including eicosadienoic acid (EDA), dihomo-γ-linoleic acid (DGLA), arachidonic acid (ARA), eicosapentaenoic acid (EPA) in various ratios in PB5 and have demonstrated here that the substrate C18:2n-6 levels required for LCPUFA biosynthesis in wildtype strain can be significantly increased by the overexpression of a heterologous LPCAT in Auxenochlorella protothecoides to effectively channel C18:1 n-9 through phospholipids. While the parent Auxenochlorella protothecoides contain endogenous LPCAT and CPT activities, these are not enough to sustain the efficient channeling of fatty acids through phospholipids needed to produce significant amounts of essential LCPUFAs. The inventors of the present invention have also demonstrated that implanting heterologous delta-9FAE and FADdelta-8 in Auxenochlorella protothecoides converts the available C18:2n-6 to EDA and DGLA, respectively. Expression of heterologous FADdelta-5 and FADdelta-17 desaturase enzymes results in the conversion of DGLA to ARA, and EPA, respectively. Eventually, Auxenochlorella protothecoides strains optimally expressing LPCAT delta-9 FAE, FADdelta-8, FADdelta-5, and FADdelta-17 will result in accumulation of oils with different compositions of essential LCPUFAs for deployment in the nutritional, pharmaceutical, and biotherapeutic markets.

The Auxenochlorella protothecoides mutant of the present invention may produce high-value essential LCPUFA oils including eicosadienoic acid (EDA), dihomo-γ-linoleic acid (DGLA), arachidonic acid (ARA), and eicosapentaenoic acid (EPA) in various ratios in cells.

Specifically, the Auxenochlorella protothecoides mutant of the present invention may produce microalgal oil comprising LCPUFA in various combinations shown below:
EDA only,
DGLA only,
ARA only,
EPA only,
EDA and DGLA,
DGLA and ARA,
DGLA and EPA,
ARA and EPA,
EDA, DGLA, ARA,
DGLA, ARA, and EPA,
EDA, DGLA, ARA, and EPA.

In addition, the Auxenochlorella protothecoides mutant of the present invention may produce microalgal oil comprising LCPUFA in various amount ratios, for example, 0.1 w/w % or more, 0.5 w/w % or more, 1 w/w % or more, 5 w/w % or more, 10 w/w % or more, 15 w/w % or more, 20 w/w % or more, 25 w/w % or more, 30 w/w % or more, 35 w/w % or more, 40 w/w % or more, 45 w/w % or more, 50 w/w % or more, 55 w/w % or more or more, 60 w/w % or more, 65 w/w % or more, 70 w/w % or more, 75 w/w % or more, 80 w/w % or more, 85 w/w % or more, 90 w/w % or more, 95 w/w % or more, 100 w/w %, 0.1 to 1 w/w %, 1 to 5 w/w %, 5 to 10 w/w %, 1 to 10 w/w %, 10 to 20 w/w %, 20 to 30 w/w %, 30 to 40 w/w %, 40 to 50 w/w %, 50 to 60 w/w %, 60 to 70 w/w %, 70 to 80 w/w %, 80 to 90 w/w %, 90 to 100 w/w %, 10 to 30 w/w %, 30 to 60 w/w %, 30 to 90 w/w %, 10 to 40 w/w %, 40 to 80 w/w %, 10 to 50 w/w %, 50 to 100 w/w %, 10 to 60 w/w %, 20 to 60 w/w %, 40 to 60 w/w %, 10 to 70 w/w %, 20 to 70 w/w %, 30 to 70 w/w %, 40 to 70 w/w %, 10 to 80 w/w %, 20 to 80 w/w %, 30 to 80 w/w %, 40 to 80 w/w %, 50 to 80 w/w %, 60 to 80 w/w %, 10 to 90 w/w %. 20 to 90 w/w %, 30 to 90 w/w %, 40 to 90 w/w %, 50 to 90 w/w %, not limited thereto.

In addition, the LCPUFA may comprise various combinations of EDA, DGLA, ARA, and EPA.

For example, the LCPUFA may comprise 100 w/w % of EDA, DGLA, ARA, or EPA.

Also, the LCPUFA may comprise

0.1 to 99.9 w/w % of EDA and 0.1 to 99.9 w/w % of DGLA,

0.1 to 99.9 w/w % of DGLA and 0.1 to 99.9 w/w % of ARA,

0.1 to 99.9 w/w % of DGLA and 0.1 to 99.9 w/w % of EPA, or

0.1 to 99.9 w/w % of ARA and 0.1 to 99.9 w/w % of EPA.

The LCPUFA may comprise

10 to 90 w/w % of EDA and 10 to 90 w/w % of DGLA,

10 to 90 w/w % of DGLA and 10 to 90 w/w % of ARA,

10 to 90 w/w % of DGLA and 10 to 90 w/w % of EPA, or

10 to 90 w/w % of ARA and 10 to 90 w/w % of EPA.

The LCPUFA may comprise

20 to 80 w/w % of EDA and 20 to 80 w/w % of DGLA,

20 to 80 w/w % of DGLA and 20 to 80 w/w % of ARA,

20 to 80 w/w % of DGLA and 20 to 80 w/w % of EPA, or

20 to 80 w/w % of ARA and 20 to 80 w/w % of EPA.

The LCPUFA may comprise

30 to 70 w/w % of EDA and 30 to 70 w/w % of DGLA,

30 to 70 w/w % of DGLA and 30 to 70 w/w % of ARA,

30 to 70 w/w % of DGLA and 30 to 70 w/w % of EPA, or

30 to 70 w/w % of ARA and 30 to 70 w/w % of EPA.

The LCPUFA may comprise

40 to 60 w/w % of EDA and 40 to 60 w/w % of DGLA,

40 to 60 w/w % of DGLA and 40 to 60 w/w % of ARA,

40 to 60 w/w % of DGLA and 40 to 60 w/w % of EPA, or

40 to 60 w/w % of ARA and 40 to 60 w/w % of EPA.

Also, the LCPUFA may comprise

0.1 to 99.8 w/w % of EDA, 0.1 to 99.8 w/w % of DGLA and 0.1 to 99.8 w/w % of ARA,

0.1 to 99.8 w/w % of DGLA, 0.1 to 99.8 w/w % of ARA and 0.1 to 99.8 w/w % of EPA,

10 to 80 w/w % of EDA, 10 to 80 w/w % of DGLA and 10 to 80 w/w % of ARA,

10 to 80 w/w % of DGLA, 10 to 80 w/w % of ARA and 10 to 80 w/w % of EPA,

20 to 60 w/w % of EDA, 20 to 60 w/w % of DGLA and 20 to 60 w/w % of ARA,

20 to 60 w/w % of DGLA, 20 to 60 w/w % of ARA and 20 to 60 w/w % of EPA,

30 to 40 w/w % of EDA, 30 to 40 w/w % of DGLA and 30 to 40 w/w % of ARA, or

30 to 40 w/w % of DGLA, 30 to 40 w/w % of ARA and 30 to 40 w/w % of EPA.

Also, the LCPUFA may comprise

0.1 to 99.7 w/w % of EDA, 0.1 to 99.7 w/w % of DGLA, 0.1 to 99.7 w/w % of ARA, and 0.1 to 99.7 w/w % of EPA,

10 to 70 w/w % of EDA, 10 to 70 w/w % of DGLA, 10 to 70 w/w % of ARA, and 10 to 70 w/w % of EPA,

15 to 55 w/w % of EDA, 15 to 55 w/w % of DGLA, 15 to 55 w/w % of ARA, and 15 to 55 w/w % of EPA,

20 to 40 w/w % of EDA, 20 to 40 w/w % of DGLA, 20 to 40 w/w % of ARA, and 20 to 40 w/w % of EPA.

The description of Auxenochlorella protothecoides, long-chain polyunsaturated fatty acid, elongase, and desaturases mentioned above in the Auxenochlorella protothecoides mutant can be equally applied to the above production process.

As an aspect for achieving the object of the present invention, the present invention provides a recombinant nucleic acid comprising a coding sequence that encodes one or more selected from a group consisting of lysophosphatidylcholine acyltransferase (LPCAT), delta-9 elongase (delta-9FAE), delta-8 desaturase (FADdelta-8), delta-5 desaturase (FADdelta-5), delta-17 desaturases (FAD delta-17), lysophosphatidic acid acyltransferase (LPAAT), cytochrome b5 (Cytb5), choline phosphotransferase (CPT) and functional equivalents thereof.

In a specific embodiment of the present invention, the above coding sequence is in operable linkage with a promoter.

As an aspect of achieving the object of the present invention, the present invention provides a recombinant vector comprising the recombinant nucleic acid.

“Vector” means a gene construct including an essential regulatory element operably linked to express a gene insert encoding a target protein in a cell of an individual and is a means for introducing a nucleic acid sequence encoding a target protein into a host cell. The vector can be at least one selected from the group consisting of various types of vectors including viral vectors such as plasmids, adenovirus vectors, retrovirus vectors, adeno-associated virus vectors, bacteriophage vectors, cosmid vectors, and YAC (Yeast Artificial Chromosome) vectors. In one example, the plasmid vector can be at least one selected from the group consisting of pBlue (e.g., pBluescript II KS(+)), pSC101, pGV1106, pACYC177, ColE1, pKT230, pME290, pBR322, pUC8/9, pUC6, pBD9, pHC79, pIJ61, pLAFR1, pHV14, pGEX series, pET series, pUC19, pUC57 and the like, the bacteriophage vector can be at least one selected from the group consisting of lambda gt4 lambda B, lambda-Charon, lambda Δz1, M13, and the like, and the viral vector can be SV40 or the like, but the present invention is not limited thereto.

The term “recombinant vector” includes cloning vectors and expression vectors containing foreign target genes. A cloning vector is a replicon, which includes an origin of replication, such as an origin of replication of a plasmid, phage, or cosmid, to which another DNA fragment can be attached so as to bring about the replication of the attached fragment.

Expression vectors have been developed so as to be used to synthesize proteins.

In the present specification, the vector is not particularly limited as long as it can express a desired gene in various host cells such as prokaryotic cells or eukaryotic cells, and perform a function of preparing the gene. However, it is desirable that the gene inserted and transferred into the vector is irreversibly fused into the genome of the host cell so that gene expression in the cell persists stably for a long period of time.

Such vectors include transcriptional and translational expression control sequences that allow a target gene to be expressed within a selected host. An expression control sequence can include a promoter for performing transcription, any operator sequence for controlling such transcription, a sequence for encoding a suitable mRNA ribosomal binding site, and a sequence for controlling the termination of transcription and translation. For example, control sequences suitable for prokaryotes include a promoter, any operator sequence, and/or a ribosomal binding site. Control sequences suitable for eukaryotic cells include promoters, terminators, and/or polyadenylation signals. The initiation codon and the termination codon are generally considered as a part of a nucleotide sequence encoding a target protein, and need to have actions in a subject when the gene construct is administered and be in frame with a coding sequence. A promoter of the vector can be constitutive or inducible. Further, in the case where the vector is a replicable expression vector, the vector can include a replication origin. In addition, enhancers, non-translated regions of the 5′ and 3′ ends of the gene of interest, selective markers (e.g., antibiotic resistance markers), or replicable units can be appropriately included. Vectors can be self-replicated or integrated into host genomic DNA.

Examples of useful expression control sequences can include early and late promoters of adenovirus, a monkey virus 40 (SV40) promoter, a mouse mammary tumor virus (MMTV) promoter, a human immunodeficiency virus (HIV) such as a long terminal repeat (LTR) promoter of HIV, molonivirus, cytomegalovirus (CMV) promoter, Epstein Barr virus (EBV) promoter, and Rous sarcoma virus (RSV) promoter, RNA polymerase II promoter, β-actin promoter, human hemoglobin promoter, and human muscle creatine promoter, lac system, trp system, TAC or TRC system, T3 and T7 promoters, a major operator and promoter site of a phage lambda, a regulatory site of a fd coat protein, promoters for phosphoglycerate kinase (PGK) or other glycol degrading enzyme, phosphatase promoters, such as a promoter of yeast acid phosphatase such as Pho5, a promoter of a yeast alpha-mating factor, and other sequences known to regulate gene expression of prokaryotic or eukaryotic cells and their viruses and combinations thereof.

In order to increase the expression level of a transformed gene in a cell, the target gene and transcription and translation expression control sequences should be operably linked to each other. Generally, the term “operably linked” means that the DNA sequences being linked are contiguous, and, in the case of a secretory leader, contiguous and present in a reading frame. For example, DNA for a pre-sequence or a secretory leader is operably linked to DNA encoding polypeptide when expressed as a pre-protein participating in the secretion of the protein, a promoter or an enhancer is operably linked to a coding sequence when affecting the transcription of the sequence; or a ribosomal binding site is operably linked to a coding sequence when affecting the transcription of the sequence, or a ribosomal binding site is operably linked to a coding sequence when arranged to facilitate translation. The linkage between these sequences is performed by ligation at a convenient restriction enzyme site. However, when the site does not exist, the linkage can be performed using a synthetic oligonucleotide adaptor or a linker according to a conventional method.

Those skilled in the art can appropriately select from among various vectors, expression control sequences, hosts, and the like suitable for the present invention, taking into account the nature of the host cell, the copy number of the vector, the ability to regulate the copy number and the expression of other protein encoded by the corresponding vector (e.g., the expression of an antibiotic marker).

The microalgal mutant provided herein may be obtained by transforming a host microalgal cell using the above recombinant vector.

As used herein, the term “transformation” means that a target gene is introduced into a host microorganism and thereby, the target gene can be replicated as a factor outside of the chromosome or by means of completion of the entire chromosome.

As the transformation method, suitable standard techniques as known in the art, such as electroporation, electroinjection, microinjection, calcium phosphate co-precipitation, calcium chloride/rubidium chloride method, retroviral infection, DEAE-dextran, cationic liposome method, polyethylene glycol-mediated uptake, gene guns and the like can be used, but are not limited thereto. At this time, the vector can be introduced in the form of a linearized vector by the digestion of a circular vector with suitable restriction enzymes.

The microalgal mutant of the present invention may grow appropriately in a growth environment (light conditions, temperature conditions, medium, etc.) capable of culturing conventional Auxenochlorella protothecoides.

The microalgal mutant of the present invention may be cultured according to the culture conditions of general Auxenochlorella protothecoides, and specifically, a culture medium capable of culturing algae under weak light conditions may be used. To culture a specific microorganism, it may include a nutrient material required for a culture target, that is, a microorganism to be cultured, and maybe mixed by adding material for a special purpose. The medium includes an all-natural medium, synthetic medium, or selective medium. The Auxenochlorella protothecoides mutant may be cultured according to a conventional culture method.

The pH of the culture medium is not particularly limited if the Auxenochlorella protothecoides may survive and grow, for example, it is viable at pH 5 or higher, specifically at pH 6 to 8.

In a specific embodiment, the microalgal mutant may be incubated under a heterotrophic growth condition for a period of time sufficient to allow the microalgal mutant to grow, wherein the heterotrophic growth condition includes a media including a carbon source, and wherein the heterotrophic growth condition further includes a low irradiance of light.

In some embodiments, the carbon source is glucose. In some embodiments, the carbon source is selected from the group consisting of a fixed carbon source, glucose, fructose, sucrose, galactose, xylose, mannose, rhamnose, N-acetylglucosamine, glycerol, floridoside, glucuronic acid, corn starch, depolymerized cellulosic material, sugar cane, sugar beet, lactose, milk whey, and molasses.

In some embodiments, the light is produced by a natural light source. In some embodiments, the light is natural sunlight. In some embodiments, the light comprises full spectrum light or a specific wavelength of light. In some embodiments, the light is produced by an artificial light source.

The Auxenochlorella protothecoides mutant of the present invention may produce high-value essential LCPUFA oils including eicosadienoic acid (EDA), dihomo-γ-linoleic acid (DGLA), arachidonic acid (ARA), eicosapentaenoic acid (EPA) in various ratios in cells, so that oils extracted from mutants of the present invention may be effectively used as raw materials for pharmaceuticals, cosmetics, food, feed, etc.

In this aspect, the present invention provides a composition comprising the oil derived from the Auxenochlorella protothecoides mutant. The composition may be a cosmetic composition, a food composition, a composition for a food additive, a feed composition, a composition for a feed additive, a pharmaceutical composition, a raw material composition for food, a raw material composition for feed, a raw material composition for pharmaceutics or a raw material composition for cosmetics.

The composition may be used as a raw material for food, feed, or pharmaceutics, and may be used as a formulation for oral administration or parenteral administration. For example, it may be used as a formulation for oral, transdermal, or injection administration. Accordingly, the composition of the present invention may be a composition for oral administration in that the composition may be orally supplied to be included in food, medicine, or feed.

In the case of compositions for oral administration may be formulated as powders, granules, tablets, pills, dragees, capsules, liquids, gels, syrups, slurries, suspensions, etc. by using methods known in the art. For example, oral preparations may be obtained by mixing the active ingredient with excipients, grinding the mixture, adding suitable additives, and processing it into a granule mixture to obtain tablets or sugar tablets. Examples of suitable excipients include sugars, including lactose, dextrose, sucrose, sorbitol, mannitol, xylitol, erythritol, and maltitol, and starches, including corn starch, wheat starch, rice starch, and potato starch, cellulose, including methylcellulose, sodium carboxymethylcellulose, and hydroxypropylmethyl-cellulose, and the like, fillers such as gelatin, polyvinylpyrrolidone, and the like may be included. In addition, cross-linked polyvinylpyrrolidone, agar, alginic acid, or sodium alginate may be added as a disintegrant if necessary.

The composition may be used for human and animal health promotion. Specifically, the mutant of the present invention has oil production ability with enhanced antioxidant pigment content, so it is not easily oxidized, and functionally, it is possible to provide an oil superior to conventional microalgae-derived vegetable oil in antioxidant activity, and it can be effectively used as a raw material for health functional food, feed, or medicine.

In addition, since the composition may be added to food or feed to achieve a special purpose use, in this respect it may be a food composition, a composition for food additives, a feed composition, or a composition for feed additives. When the composition is used in feed or food, it is possible to maintain or enhance body health by pigments and lipids produced by the mutant and accumulated in cells.

In the present invention, “additive” is included as long as it is a material added to food or feed other than the main raw material, and specifically, it may be an effective active material having functionality in food or feed.

In the present invention, the composition for feed may be prepared in the form of fermented feed, compounded feed, pellet form, and silage. The fermented feed may include a functional oil derived from the mutant of the present invention, and additionally include various microorganisms or enzymes.

The composition is mixed with a carrier commonly used in the food or pharmaceutical field, such as tablets, troches, capsules, elixirs, syrups, and powders. It can be prepared and administered in the form of powder, suspension, or granules. As the carrier, binders, lubricants, disintegrants, excipients, solubilizers, dispersants, stabilizers, suspending agents, and the like may be used. The administration method may be an oral, or parenteral method, but preferably oral administration. In addition, the dosage may be appropriately selected according to the absorption of the active ingredient in the body, the inactivation rate and excretion rate, the age, sex, condition of the subject, and the like. The pH of the composition can be easily changed according to the manufacturing conditions of the drug, food, cosmetics, etc. in which the composition is used.

The composition may include 0.001 to 99.99% by weight, preferably 0.1 to 99% by weight of any one selected from the group consisting of the microalgal mutants of the present invention, the culture of the mutants, the dried product of the mutant, or the culture thereof, and the extract of the mutant or the culture thereof, and the functional oil derived from the mutant, based on the total weight of the composition, and the method of using the composition and the content of the active ingredient may be appropriately adjusted according to the purpose of use.

The mutant may be included in the composition in its own or dried form, and the culture of the mutant may be included in the composition in a concentrated or dried form. In addition, the dried product refers to the dried form of the mutant or its culture and may be in the form of a powder prepared by freeze-drying or the like.

In addition, the extract means that obtained by extraction from the mutant of the present invention, its culture medium or its dried product, an extract using a solvent, etc. Thus, the mutant of the present invention includes those obtained by crushing the mutant of the present invention. Specifically, the high-value essential LCPUFA oils accumulated in the cells of the mutant of the present invention may be extracted and separated by a physical or chemical method.

In addition, the method for producing high-value essential LCPUFA oils according to the present invention may include culturing the mutant of the present invention. In addition, the production method may further include; after the culturing step, isolating the mutant of the present invention from the culture.

As an aspect for achieving the object of the present invention, the present invention provides a process for the production of long-chain polyunsaturated fatty acid in recombinant Auxenochlorella protothecoides which comprises the following steps:

a) introducing a combination of at least one nucleic acid sequence which encodes elongases and at least one nucleic acid sequence which encodes desaturases into Auxenochlorella protothecoides to prepare the recombinant Auxenochlorella protothecoides; and

b) culturing the recombinant Auxenochlorella protothecoides to a long-chain polyunsaturated fatty acid.

The culture may be performed in a medium of pH 5.0 to 8.0 conditions. In addition, it may be carried out under a weak light condition, specifically, a light intensity condition in the range of 0.1-1, 1-3, or 3-5 μmol photons/m² s, not limited thereto. In other embodiments, it may be carried out under a heterotrophic growth condition where no light source is administered. In other embodiments, it may be carried out under a phototrophic condition with light intensity from 30-500 μmol photons/m² s, not limited thereto.

The production method may further include, in addition to the culturing step, a concentration step to increase the content of algae after culturing, and a drying step of drying by further reducing the moisture of the algae that has undergone the concentration step. However, the concentration step or the drying step is not necessarily required, and in general, the concentration and drying method commonly used in the field to which the present invention pertains, and it can be carried out using a machine.

The production method may further include the step of extraction of oil and purifying the oil isolated from the culture, which may be performed by a conventional purification method in the art to which the present invention pertains.

### Detailed Description of the Embodiments

Hereinafter, the present invention will be explained in detail through Examples and Experimental Examples, but these Examples and Experimental Examples are presented only as the illustration of the present invention, and the scope of the present invention is not limited thereby.

### Example 1. Production of Auxenochlorella protothecoides Strains with Increased Levels of Eicosadienoic Acid (C20:2 n6)

In this example, we generated strains that produce eicosadienoic acid (C20:2n-6) by adding two carbons to linoleic acid. To accomplish this, we made a DNA construct, pPB0177, that allowed targeted integration of the transforming DNA via homologous recombination at the D-aspartate oxidase 1 (DAO1) locus within the A. protothecoides genome. The construct contained a heterologous fatty acid elongase delta-9 from E. gracilis (Eg-delta-9FAE; Accession number: CAT16687) codon-optimized for optimal expression in A. protothecoides. Construct pPB0177 introduced for expression in A. protothecoides can be written as pPB0177: ApDAO1:: ApHUP1-AtTHIC-ApHSP90: ApSAD2v1-Eg-delta-9FAE-ApSAD2v1::ApDAO1

The sequence of the transforming DNA construct pPB0177 is shown below in FIG. 3. Relevant restriction sites in the construct are indicated in lowercase bold text. EcoRV restriction endonuclease site used to generate linear DNA and for cloning is indicated in lowercase bold and delimits the 5′ and 3′ ends of the transforming DNA. Underlined uppercase text at the 5′ and 3′ flanks of the construct represent genomic DNA from A. protothecoides PB5 that enable targeted integration of the transforming DNA via homologous recombination at the DAO1 locus. Proceeding in the 5′ to 3′ direction, the A. protothecoides HUP1 (hexose/H+ symporter) promoter (Ap-HUP1) driving the expression of the A. thaliana ThiaminC gene (At-THIC), codon-optimized for expression in A. protothecoides and encoding 4-amino-5-hydroxymethyl-2-methylpyrimidine synthase activity, thereby permitting the strain to grow in the absence of exogenous thiamine, is indicated in lowercase, boxed text. The initiator ATG and terminator TGA for At-THIC are indicated in uppercase italics, while the coding region is indicated with lowercase italics. The terminator region of the A. protothecoides heat shock protein 90 (Ap-HSP90) gene is indicated by small capitals followed by an endogenous A. protothecoides stearoyl ACP desaturase (ApSAD2v1) promoter indicated by the lowercase boxed text. The Initiator ATG and terminator TGA codons of the E. gracilis fatty acid elongase delta-9 (Eg-delta-9FAE) are indicated by uppercase italics, while the remainder of the gene is indicated in lowercase italics. The endogenous A. protothecoides stearoyl ACP desaturase terminator region Ap-SAD2v1 is indicated in small capitals followed by A. protothecoides PB5 DAO1 genomic region indicated by the underlined uppercase text. The final construct was sequenced to ensure correct reading frames and targeting sequences.

In addition to the Eg-delta-9FAE gene, FAE genes from Isochrysis galbana (Ig-ASE1 and Ig-ASE2; Accession No's: AF390174_1 and ADD51571) and Pavlova pinguis (Ppin-delta-9FAE; Accession No: ADN94475) were constructed for expression in A. protothecoides PB5. The constructs harboring these genes can be written as

pPB0178: ApDAO1::ApHUP1-AtTHIC-ApHSP90:ApSAD2v1-IgASE1-delta-9FAE-ApSAD2v1::ApDAO1

pPB0179: ApDAO1::ApHUP1-AtTHIC-ApHSP90:ApSAD2v1-IgASE2-delta-9FAE-ApSAD2v1::ApDAO1

pPB0180: ApDAO1::ApHUP1-AtTHIC-ApHSP90:ApSAD2v1-Ppin-delta-9FAE-ApSAD2v1::ApDAO1

All these constructs have the same vector backbone; selectable marker, promoters, and 3′ untranslated region (UTR) as pPB0177, differing only in the respective delta-9FAE genes. Relevant restriction sites in these constructs are also the same as in pPB0177. FIGS. 4-6 indicate the sequence of Ig-ASE1-delta-9FAE, Ig-ASE2-delta-9FAE, and Ppin-delta-9FAE in lowercase with the initiator ATG and terminator TGA codons in uppercase italics.

To determine their impact on fatty acid profiles, the above constructs, containing various heterologous fatty acid elongase genes, driven by the ApSAD2v1 promoter, were transformed independently into PB5 and primary transformants were selected on growth media without thiamine. Single clonally purified colonies were grown under standard lipid production conditions in shake flasks. The fatty acid profiles from representative derivative lines arising out of the transformation of wildtype A. protothecoides PB5 with pPB0177 (lines PB5; 177-8 and PB5:177-6), pPB0178 (lines PB5; 178-2 178-8 and 178-12), pPB0179 (lines PB5; 179-4), and pPB0180 (lines PB5; 180-9 and PB5; 180-12) are shown in Table 1.

TABLE 1
Fatty acid profiles as a percentage of total fatty acids from representative derivative
PB5 lines transformed with plasmids pPB0177, pPB0178, pPB0179, and pPB0180.

| | C16:0 | C18:0 | C18:1n-9 | C18:2n-6 | C18:3n-3 | C20:2n-6 |
|---|---|---|---|---|---|---|
| Sample Name | Palmitic | Stearic | Oleic | Linoleic | ALA | EDA |
| PB5 Wildtype | 12.87 | 2.63 | 67.53 | 13.02 | 2.07 | — |
| PB5; 177-6 | 12.76 | 2.56 | 67.37 | 13.36 | 1.95 | — |
| PB5; 177-8 | 13.31 | 2.57 | 68.89 | 10.03 | | 3.26 |
| PB5; 178-2 | 13.34 | 2.54 | 67.72 | 12.4 | 1.96 | — |
| PB5; 178-8 | 13.12 | 3.05 | 68.53 | 10.00 | | 3.37 |
| PB5; 178-12 | 12.10 | 2.15 | 68.88 | 13.10 | | 1.98 |
| PB5; 179-4 | 12.97 | 2.53 | 68.24 | 8.05 | | 6.18 |
| PB5; 180-9 | 11.87 | 3.07 | 70.59 | 6.48 | | 6.25 |
| PB5; 180-12 | 12.96 | 2.49 | 68.89 | 6.91 | | 6.75 |

Transformed A. protothecoides PB5 derivative lines expressing heterologous Eg-delta-9FAE (PB5; 177-8) showed a new fatty acid peak in lipid profiles corresponding to EDA (C20:2n-6) over the control PB5. The increase in C20:2n-6 levels was concomitant with the diminution of C18:2n-6, demonstrating that the heterologous Eg-delta-9FAE enzyme activity in PB5 adds two carbos to C18:2n-6 to elongate it to C20:2n-6. A similar pattern in C20:2n-6 accumulation with a corresponding decrease in C18:2n-6 levels resulted in strains expressing Ig-ASE1-delta-9FAE (lines PB5; 178-8 and PB5; 178-12), Ig-ASE2-delta-9FAE (lines PB5; 179-4), or Ppin-delta-9FAE (lines PB5; 180-9 and PB5; 180-12). PB5; 179-4 and PB5; 180-12 were banked as Phycoil engineered strains (PES) PES-01 and PES-02, respectively, and used as parent strains for subsequent transformations.

### Example 2. Heterologous Expression of a Fatty Acid Desaturase Delta-8 in Phycoil Strains PES-01 or PES-02 Results in the Production of Dihomo-γ-Linoleic Acid or DGLA (C20:3n-6

After having successfully elongated linoleic acid (C18:2n-6) to eicosadienoic acid (C20:2n-6), we next attempted to desaturate the newly accumulated C20:2n-6 to C20:3n-6 (dihomo-γ-linoleic acid or DGLA). To accomplish this, we made a DNA construct, pPB0238, that allowed targeted integration of the transforming DNA, via homologous recombination, at the THI4 (Thiamine biosynthesis 4) locus within the A. protothecoides (PB5) genome. The construct contained a heterologous fatty acid desaturase delta-8 from I. galbana (Ig-FADdelta-8; Accession No: AFB82640) codon-optimized for optimal expression in PB5. Construct pPB0238 introduced for expression in PES-01 or PES-02 can be written as

pPB0238: ApTH14::ApSAD2v1-Ig-FADdelta-8-ApSAD2v1:CrTUB2-ScSUC2-ApPGH::ApTH14

The sequence of the transforming construct pPB0238 is provided in FIG. 7.

Relevant restriction sites in the construct are indicated in lowercase, bold, and are from 5′-3′ HindIII, XbaI, SpeI, Xhol, and HindIII, respectively. HindIII restriction endonuclease site used to generate linear DNA delimits the 5′ and 3′ ends of the transforming DNA. Underlined uppercase text at the 5′ and 3′ flanks of the construct represent genomic DNA from A. protothecoides PB5 that enable targeted integration of the transforming DNA via homologous recombination at the THI4 locus within the A. protothecoides PB5 genome. Proceeding in the 5′ to 3′ direction, the A. protothecoides stearoyl ACP desaturase (ApSAD2v1) promoter, driving the expression of codon-optimized Ig-FADdelta-8, is indicated by the lowercase boxed text. The initiator ATG and terminator TGA for Ig-FADdelta-8 are indicated in uppercase italics, while the coding region is indicated in lowercase italics. The terminator region of the A. protothecoides stearoyl ACP desaturase (ApSAD2v1 terminator) gene is indicated by small capitals followed by Chlamydomonas reinhardtii beta-tubulin 2 (CrTUB2) promoter in lowercase boxed text, driving expression of Saccharomyces cerevisiae SUC2 gene (ScSUC2, codon-optimized for expression in A. protothecoides and encoding sucrose invertase, thereby enabling the strain to utilize exogenous sucrose). The initiator ATG and terminator TGA for ScSUC2 are indicated in uppercase italics, while the coding region is indicated in lowercase italics. The terminator region of the A. protothecoides enolase gene (ApPGH) gene is indicated in small capitals followed by A. protothecoides PB5 THI4 genomic region indicated by the underlined uppercase text. The final construct was sequenced to ensure the correct reading frames and targeting sequences.

In addition to the Ig-FADdelta-8 gene, a FADdelta-8 gene from P salina was constructed for expression in PES-01. The construct pPB0239 harboring Ps-FADdelta-8 gene can be written as

pPB0239: ApTH14::ApSAD2v1-Ps-FADdelta-8-ApSAD2v1:CrTUB2-ScSUC2-ApPGH::ApTH14

The above construct has the same vector backbone, selectable marker, promoters, and 3′ UTR as pPB0238 differing only in the respective FADdelta-8 gene. Relevant restriction sites in the construct are also the same as in pPB0238. FIG. 8 indicates the sequence of Ps-FADdelta-8 in lowercase with the initiator ATG and terminator TGA codons in uppercase italics contained in pPB0239.

pPB0238 and pPB0239 were transformed into Phycoil strain PES-01, and primary transformants were selected on sucrose-containing growth media without thiamine. Single clonally purified colonies were grown under standard lipid production conditions in shake flasks. The fatty acid profiles of lipids from shake flask assays of representative derivative lines containing the pPB0238 (PES-01; 238-6) and pPB0239 (PES-01; 239-1 and PES-01; 239-2) construct are shown in Table 2.

TABLE 2
Fatty acid profiles as a percentage of total fatty acids for Phycoil PES-01 strain
transformed with pPB0238 (PES-01; 238-6) and pPB0239 (PES-01; 239-1,
PES-01; 239-2)

| Sample | C16:0 | C18:0 | C18:1n-9 | C18:2n-6 | C18:3n-3 | C20:2n-6 | C20:3n-6 |
|---|---|---|---|---|---|---|---|
| name | Palmitic | Stearic | Oleic | Linoleic | ALA | EDA | DGLA |
| PES-01 | 11.98 | 2.17 | 66.13 | 7.80 | 0.86 | 6.95 | |
| Parent | | | | | | | |
| PES-01; | 11.51 | 2.08 | 53.65 | 7.64 | 1.24 | 5.67 | 3.38 |
| 238-6 | | | | | | | |
| PES-01; | 11.88 | 2.58 | 62.65 | 8.97 | 1.13 | 4.76 | 3.42 |
| 239-1 | | | | | | | |
| PES-01; | 11.52 | 2.28 | 65.09 | 7.13 | 1.25 | 4.99 | 3.15 |
| 239-2 | | | | | | | |

More than half of the eicosadienoic acid (EDA; C20:2n-6) in parent strain PES-01 (expressing IgASE2-delta-9FAE) was desaturated to dihomo-γ-linoleic acid (DGLA; C20:3n-6) in derivative lines PES-01; 238-6 (expressing Ig-FADdelta-8) or PES-01; 239-1 and PES-01; 239-2 (expressing Ps-FADdelta-8). Additionally, desaturation of EDA (C20:2n-6) to DGLA (C20:3n-6) plausibly created a feedback loop with increased endogenous LPCAT activity resulting in more oleic acid (C18:1n-9) being channeled through phospholipids and becoming available for further desaturation to linoleic acid, elongation to EDA, and finally desaturation to DGLA by endogenous FAD2 delta-12, heterologous delta-9FAE, and heterologous FADdelta-8 enzymes, respectively. Reduction in C18:1 n-9 level (˜66% PES-01 parent strains vs. 53.65% in PES-01; 238-6, 62.65% in PES-01; 239-1, and 65.09% in PES-01; 239-2) was concomitant with an overall increase in C18:2n-6, C20:2n-6, and C20:3n-6 levels. PES-01; 238-6 and PES-01; 239-1 were banked as Phycoil engineered strains PES-03 and PES-04, respectively, and used as parent strains for subsequent transformations.

### Example 3. Modulating the Lands Cycle Enzyme Activities Boosts the Production of Linoleic (C18:2n-6) and Eicosadienoic (C20:2n6) Acids

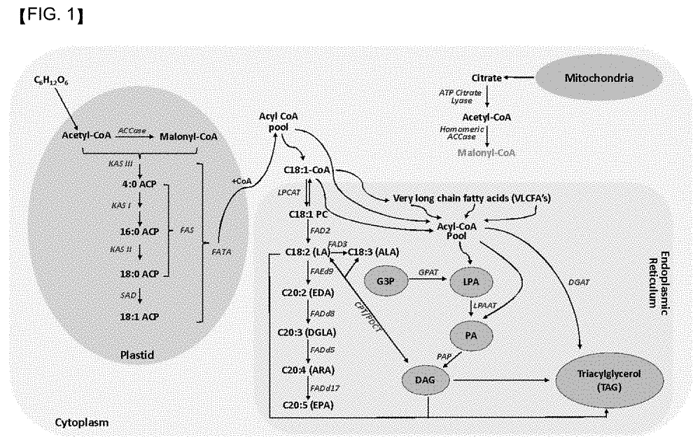

Expression of heterologous elongases in A. protothecoides PB5, as described in example 1, led to the elongation of C18:2n-6 (linoleic acid) to produce C20:2n-6 (EDA). In several strains, nearly 50% of the available C18:2n-6 was elongated to C20:2n-6. The Δ9 double bond in C18:1 n-9 is introduced by the stearoyl-ACP desaturases (SADs) in the plastids. Formation of the Δ12 double bond in C18:2n-6, catalyzed by FAD2, occurs on the phospholipid membranes in the endoplasmic reticulum. The relatively low abundance of C18:2n-6 in wild-type A. protothecoides storage lipid results from the competition between the acyltransferases of the Kennedy pathway (for the formation of TAG) and the enzymes of the Lands cycle, which control the exchange of fatty acids between diacylglycerol (DAG) and membrane phospholipids (FIG. 1). Wildtype A. protothecoides PB5 strains, when cultured under lipid production conditions produce final oil with around 13% C18:2n-6 levels and point towards functional but perhaps non-optimal endogenous LPCAT and downstream DAG-CPT/PDCT enzyme activities in our host. We posited that increasing the available pool of C18:2n-6 on the phospholipids would make more substrate available for elongation by delta-9 elongases and result in even more eicosadienoic acid in resultant strains.

In order to test this hypothesis, we introduced constructs overexpressing Arabidopsis thaliana LPCAT1 (Accession No: NP_172724)—encoding lysophosphatidylcholine acyltransferase (pPB0234), PDCT (Accession No: NP_566527)—encoding phosphatidyl-choline diacylglycerol cholinephosphotransferase (pPB0214) or a combination of both genes (pPB0222), into Phycoil strains PES-01 (expressing the Ig-ASE2-delta-9FAE) and PES-02 (expressing Ppin-delta-9FAE).

Constructs pPB0214, pPB0234, and pPB0222, targeting At-LPCAT1, At-PDCT and a combination of At-PDCT and At-LPCAT1 along with selection marker ScSUC2 into second allele of D-aspartate oxidase 1 (DAO1) genomic locus, in PES-01 or PES-02 can be written as

pPB0234: ApDAO1::CrTUB2-ScSUC2-ApPGH:ApAMT2v1-At-LPCAT1-ApSAD2v1::ApDAO1

pPB0214: ApDAO1::CrTUB2-ScSUC2-ApPGH:ApAMT1-At-PDCT-ApPGK1::ApDAO1

pPB0222: ApDAO1::CrTUB2-ScSUC2-ApPGH:ApAMT1-At-PDCT-ApPGK1:ApAMT2v1-At-LPCAT1 ApSAD2v1::ApDAO1

The sequence of the transforming construct pPB0234 is provided in FIG. 9.

Relevant restriction sites in the construct are indicated in lowercase bold and are from 5′-3′ EcoRV, SpeI, NotI, AfIII, and EcoRV, respectively. EcoRV restriction endonuclease site used to generate linear DNA and for cloning is indicated in lowercase bold and delimits the 5′ and 3′ ends of the transforming DNA. Underlined, uppercase sequences represent genomic DNA from A. protothecoides PB5 that permit targeted integration of the transforming DNA at the DAO1 locus via homologous recombination. Proceeding from 5′ to 3′, the selection cassette contains the C. reinhardtii beta-tubulin 2 (CrTUB2) promoter in lowercase, boxed text, driving expression of Saccharomyces cerevisiae SUC2 gene (ScSUC2), codon-optimized for expression in A. protothecoides and encoding sucrose invertase, thereby enabling the strain to utilize exogenous sucrose. The initiator ATG and terminator TGA for ScSUC2 are indicated in uppercase italics while the rest of the sequence is indicated in lowercase italics. The terminator region of the A. protothecoides enolase gene (ApPGH) gene is indicated in small capitals followed by A. protothecoides ammonium transporter 2 (ApAMT2v1) promoter (indicated as small case boxed text) driving the expression of codon-optimized At-LPCAT1. The initiator ATG and terminator TGA for At-LPCAT1 are indicated in uppercase italics, while the coding region is indicated in lowercase italics. The ApSAD2v1 terminator region is indicated by small capitals followed by the A. protothecoides PB5 genomic region indicated by the underlined uppercase text. The final construct was sequenced to ensure correct reading frames and targeting sequences.

pPB0214 has the same vector backbone and selectable marker cassette as pPB0234, differing only in the Lands cycle enzyme being tested and the promoter and 3′UTR being used to drive its expression. Relevant restriction sites in the construct are also the same as in pPB0234. In pPB0214, we tested the function of the At-PDCT gene driven by the A. protothecoides ammonium transporter 1 (ApAMT1) promoter and A. protothecoides phosphoglycerate kinase 1 (ApPGK1 terminator) as the terminator sequence. The sequence of the ApAMT1-At-PDCT-ApPGK cassette contained in pPB0214 is provided in FIG. 10. A. protothecoides ammonium transporter 2 (ApAMT2v1) promoter is indicated in small case boxed text and drives the expression of codon-optimized At-LPCAT1. The initiator ATG and terminator TGA for At-LPCAT1 are indicated in uppercase italics, while the coding region is indicated in lowercase italics. The terminator region of the ApPGK1 is indicated by small capitals. NotI and AfIII restriction sites, at the beginning and end of the cassette, are depicted in lowercase bold.

pPB0222 has the same vector backbone, selectable marker cassette, and relevant restriction sites like pPB0234 and pPB0214 described above. However, it differs from both constructs in that we combined both At-PDCT and At-LPCAT1 cassettes from pPB0234 and pPB0214 into pPB0222.

The sequence of ApAMT1-At-PDCT-ApPGK1:ApAMT2v1-At-LPCAT1-ApSAD2v1 cassettes contained in pPB0222 is provided in FIG. 11. NotI restriction site, in the beginning, and AfIII restriction sites in the middle and end, are depicted in lowercase bold. A. protothecoides ammonium transporter 1 (ApAMT1) promoter is indicated as small case boxed text and drives the expression of codon-optimized At-PDCT The initiator ATG and terminator TGA for At-PDCT are indicated in uppercase italics, while the coding region is indicated with lowercase italics. The ApPGK terminator region is indicated by small capitals followed by A. protothecoides ammonium transporter 2 (ApAMT2v1) promoter (indicated as small case boxed text) driving the expression of codon-optimized At-LPCAT1. The initiator ATG and terminator TGA for At-LPCAT1 are indicated in uppercase italics, while the coding region is indicated in lowercase italics. The ApSAD2v1 terminator region is indicated by small capitals. The final construct was sequenced to ensure correct reading frames and targeting sequences.

pPB0234, pPB0214, and pPB0222 were transformed into strain PES-01 (expressing Ig-ASE2 delta-9 FAE), and primary transformants were selected on sucrose-containing growth media without thiamine. Single clonally purified colonies were grown under standard lipid production conditions in shake flasks. The fatty acid profiles of lipids from shake flask assays of representative lines transformed with plasmids pPB0234 (lines PES-01; 234-1 and PES-01; 234-2), pPB0214 (lines PES-01; 214-1 and PES-01; 214-6), and pPB0222 (lines PES-01; 222-1 and PES-01; 222-2) are shown in Tables 3.

TABLE 3
Fatty acid profiles as a percentage of total fatty acids for parental strains (PES-01
and PES-02) and representative derivative transformants containing pPB0234 (PES-01;
234-1 and PES-01; 234-2), pPB0214 (lines PES-01; 214-1 and PES-01; 214-6), and
pPB0222 (lines PES-01; 222-1 and PES-01; 222-2) constructs.

| | C16:0 | C18:0 | C18:1n-9 | C18:2n-6 | C18:3n-3 | C20:2n-6 |
|---|---|---|---|---|---|---|
| Sample name | Palmitic | Stearic | Oleic | Linoleic | ALA | EDA |
| PES-01 | 12.42 | 3.26 | 67.71 | 6.15 | 0.63 | 8.90 |
| Parent | | | | | | |
| PES-01; 234-1 | 10.93 | 4.12 | 52.50 | 14.26 | 1.03 | 16.80 |
| PES-01; 234-2 | 11.32 | 3.75 | 51.93 | 14.27 | 1.06 | 17.34 |
| PES-01; 214-1 | 11.32 | 3.88 | 49.89 | 23.96 | 0.99 | 8.98 |
| PES-01; 214-6 | 11.36 | 3.69 | 50.65 | 23.53 | 0.98 | 9.14 |
| PES-01; 222-1 | 12.01 | 1.91 | 40.55 | 27.41 | 1.64 | 12.03 |
| PES-01; 222-2 | 12.75 | 2.11 | 39.65 | 26.66 | 1.63 | 11.23 |

C18:2n-6 (linoleic acid) levels, being 6.15% in the PES-01 parental line, increased by 2- or more folds in derivative transformants expressing At-LPCAT1 (14.26% in line PES-01; 234-1 and 14.27% and 14.27% in line PES-01; 234-2), indicating that expression of At-LPCAT1 during lipid production significantly increases the channeling of C18:1n-9 to phospholipid membranes where they become available for desaturation by endogenous FAD2 enzyme and are converted into C18:2n-6. Since the PES-01 also expresses Ig-delta-9FAE elongase, a significant portion of the available C18:2n-6 was elongated to C20:2n-6 (EDA) resulting in a 2-fold increase over the amount seen in PES-01 (16.8% and 17.34% EDA in PES-01; 234-1 and PES-01; 234-2 vs ˜8.9% in PES-01). Combined C18:2n-6 and C20:2n-6 increased from around 14% in parent strain PES-01 to more than 31% in PES-01; 234-1 and PES-01; 234-2 demonstrating the positive effect of increased LPCAT activity in these strains.

A considerably different profile emerged in derivative transformed lines expressing At-PDCT (lines PES-01; 214-1 and PES-01; 214-6) transformed into the parent PES-01. While C18:2n-6 content increased by more than 3-fold (23.96% and 23.53% in PES-01; 214-1 and PES-01; 214-6 vs 6.15% in parent PES-01), there was almost no increase in C20:2n-6 EDA content in transformed strains (8.98% and 9.14% in PES-01; 214-1 and PES-01; 214-6 vs the 8.9% in PES-01 parent). Endogenous choline-phosphotransferase activity (encoded by the CPT gene) modulates symmetrical interconversion of C18:2n-6 (and C18:3n-3) between phosphatidylcholine (PC) and diacylglycerol (DAG). Like CPT, At-PDCT modulates symmetrical interconversion of C18:2n-6 (and C18:3n-3) between phosphatidylcholine (PC) and diacylglycerol (DAG). The fact that At-PDCT results in an increase in C18:2n-6 levels in TAGs suggest that this enzyme complements the endogenous CPT activity and efficiently channels C18:2n-6 out of phospholipids into DAGs. DAGs are eventually converted into TAGs by Kennedy pathway acyltransferases. Increased PC to DAG channeling of C18:2n-6 plausibly results in more available space on phospholipids and thus more channeling of C18:1n-9 into phospholipids (driven by endogenous LPCAT activity in our organism). Conceivably, the combined endogenous CPT and heterologous At-PDCT enzyme activities are so efficient that there is not enough C18:2n-6 substrate available for further elongation to C20:2n-6 (EDA) by Ig-delta-9FAE in PES-01. As seen for At-LPCAT1 expressing strains above, combined C18:2n-6 and C20:2n-6 increased from around 14% in parent strains (PES-01 or PES-02) to 32.88% in PES-01; 214-1 and 32.67% in PES-01; 214-6 lines demonstrating the effect of increased PDCT activity in these strains.

Increased C18:2n-6 and C20:2n-6 content, in derivative representative lines PES-01; 234-1 and PES-01; 234-2 (expressing At-LPCAT1) or PES-01; 214-1 and PES-01; 214-6 (expressing At-PDCT) was concomitant with a corresponding decrease in C18:1 n-9 levels (52.5% and 51.93% in PES-01; 234-1 and PES-01; 234-2 and 49.89% and 50.65% in PES-01; 214-1 and PES-01; 214-6 compared to 67.7% in PES-01). We posited that the co-expression of At-LPCAT1 and At-PDCT would result in an even more efficient channeling of C18:1 n-9 through phospholipids and incorporation of C18:2n-6 and C20:2n-6 into DAGs. As expected, derivative representative lines PES-01; 222-1 and PES-01; 222-2, expressing both At-PDCT and At-LPCAT1, showed an even greater diminution in C18:1n-9 level (40.55% in PES-01; 222-1 and 39.65% in PES-01; 222-2) compared to lines expressing either of the two enzymes (52.5% and 51.93% in PES-01; 234-1 and PES-01; 234-2 and 49.89% and 50.65% in PES-01; 214-1 and PES-01; 214-6 lines). This extra C18:1n-9 channeled into phospholipids was desaturated by endogenous FAD2 enzyme resulting in a nearly 4-fold increase in C18:2n-6 (27.41% in PES-01; 222-1 and 26.66% in PES-01; 222-2 vs 6.15% in the PES-01 parent) and 1.5-fold increase in C20:2n-6 (12.03% in PES-01; 222-1 and 11.23% in PES-01; 222-2 vs 8.9% in PES-01 parent). However, as described above for derivative lines PES-01; 214-1 and PES-01; 214-6, the majority of C18:2n-6 was unavailable for further elongation because of its efficient transfer to DAGs by boosted phosphotransferase activity of heterologous At-PDCT along with endogenous CPT activity.

Taken together, the above data suggest that endogenous LPCAT and CPT activities are fairly limited in our organism and supplementing them with heterologous At-LPCAT1and At-PDCT enzymes maximizes the channeling of C18:1 n-9 through phospholipids for further desaturation and incorporation into DAGs and TAGs. The data also suggests that boosting choline-phosphotransferase activity, by expression of a heterologous PDCT enzyme from A thaliana, might be counterproductive to produce LCPUFA biosynthesis (EDA and beyond) in our organism as it ties up the substrate C18:2n-6 into DAGs, and ultimately TAGs, thus making them unavailable for further modification by elongases.

PES-01; 214-1, PES-01; 222-1, and PES-01; 234-1 were banked as Phycoil engineered strains PES-05, PES-06, and PES-07 respectively, and used as parent strains for subsequent transformations.

### Example 4. Combinatorial Expression of Ig-Delta-9FAE, at-LPCAT1, and Ig-FADdelta-8 or Ps-FAD Delta-8 at an Upregulated ApACCase Locus Further Optimizes DGLA Production

In examples 2 and 3, described above, we tested the functions of FADdelta-8 enzymes from I. galbana (Ig-FADdelta-8) or P. salina (Ps-FADdelta-8) in Phycoil engineered strains PES-01 expressing a heterologous delta-9FAE from I. galbana (Ig-delta-9FAE). We also tested the functions of A. thaliana phosphatidylcholine diacylglycerol choline phosphotransferase (At-PDCT) and lyso-PC acyltransferase (At-LPCAT1) in the strain PES-01. In the current example, we aspired to combine activities of various enzymes tested above besides upregulating A. protothecoides ACCase gene expression to further optimize the production of linoleic (C18:2n-6), EDA (C20:2n-6), and DGLA (C20:3n-6) fatty acids.

Extension of fatty acids beyond C18, in higher plants and microalgae, requires the coordinated action of four key cytosolic/ER enzymes—a Ketoacyl Co-A synthase (KCS aka fatty acid elongase, FAE), a Ketoacyl-CoA Reductase (KCR), a Hydroxyacyl-CoA Hydratase (HACD) and an Enoyl-CoA Reductase (ECR). Each elongation reaction condenses two carbons at a time from malonyl-CoA to an acyl group, followed by reduction, dehydration, and a final reduction reaction. KCS (or FAE) catalyzes the condensation of malonyl-CoA with an acyl primer. Malonyl-CoA itself is generated through irreversible carboxylation of cytosolic acetyl-CoA by the action of multidomain cytosolic homomeric acetyl-coenzyme A carboxylase (ACCase). For efficient and sustained fatty acid elongation, the unavailability of ample malonyl-CoA can potentially become a bottleneck. Besides Malonyl-CoA is also used to produce flavonoids, anthocyanins, malonate D-amino-acids, and malonyl-amino cyclopropane-carboxylic acid, which may further decrease its availability for elongation. Using the bioinformatics approach, we identified both alleles for ApACCase in A. protothecoides. ApACCase-1 encodes a 2390 amino acid protein while ApACCase-2 encodes a 2414 amino acid protein (size difference in alleles is most likely due to bioinformatics mis-assembly). Given the large size of the protein, we decided to upregulate the expression of the ApACCase to provide additional malonyl-CoA and try to further boost the elongation of C18:2n-6 to C20:2n-6. This was accomplished by hijacking the endogenous ApACCase promoter with the A. protothecoides ammonium transporter 1 (ApAMT1) promoter in various Phycoil-engineered strains. The “promoter hijack” was accomplished by inserting the various heterologous gene cassettes together with the ApAMT1 promoter between the endogenous ApACCase-2 promoter and the initiation codon of the ApACCase-2 gene in PES-04, PES-05, and PES-07 strains.

To accomplish the objectives stated above, we made a construct pPB0265 containing the At-LPCAT1 gene for transformation into Phycoil strain PES-04 (Elongase-FADdelta-8 strain). The constructs pPB0265, targeted to A. protothecoides ApACCase locus, with ApAMT1 promoter at the 3′ end can be written as:

pPB0265: ApACCase::ApPGK1-1p-neoR(s)-ApPGK1:ApAMT2v1p-At-LPCAT1:ApSAD2v1:ApAMT1p::ApACCase The sequence of the transforming DNA construct pPB0265 is shown below in FIG. 12.

Relevant restriction sites in the construct are indicated in lowercase, bold, and are from 5′-3′ HindIII, KpnI, SpeI, XbaI, and HindIII, respectively. HindIII sites delimit the 5′ and 3′ ends of the transforming DNA. Underlined, uppercase sequences represent genomic DNA from A. protothecoides PB5 that permit targeted integration of heterologous gene cassettes and ApAMT1 promoter at the ApACCase-2 locus via homologous recombination. Proceeding from 5′ to 3′, the selection cassette contains the A. protothecoides phosphoglycerate kinase 1 (ApPGK1) promoter in lowercase, boxed text, driving expression of neomycin phosphotransferase II gene (Neo, codon-optimized for expression in A. protothecoides and encoding neomycin phosphotransferase II, thereby enabling the strain to grow on aminoglycoside antibiotic G418). The initiator ATG and terminator TGA for Neo are indicated in uppercase italics while the rest of the sequence is indicated in lowercase italics. The terminator region of the A. protothecoides phosphoglycerate kinase 1 (ApPGK1 terminator) is indicated by small capitals followed by A. protothecoides ammonium transporter 2 (ApAMT2v1) promoter (indicated as small case boxed text) driving the expression of codon-optimized A. thaliana LPCAT1 (At-LPCAT1) gene. The initiator ATG and terminator TGA for At-LPCAT1 are indicated in uppercase italics, while the coding region is indicated in lowercase italics. The A. protothecoides stearoyl ACP desaturase terminator (ApSAD2v1 terminator) region is indicated by small capitals followed by the A. protothecoides ammonium transporter 1 (ApAMT1) promoter. Immediately following the ApAMT1 promoter is the ApACCase genomic region indicated by underlined uppercase text with the ATG initiator codon of the ApACCase gene in bold letters. The final construct was sequenced to ensure correct reading frames and targeting sequences.

We also made constructs pPB0266 and pPB0267 containing I. galbana and P salina FADdelta-8 genes respectively for transformation into Phycoil strain PES-05 (Elongase-PDCT strain) or PES-07 (Elongase-LPCAT1 strain). The constructs pPB0266 and pPB0267, targeted to ApACCase locus, with ApAMT1 promoter at the 3′ end can be written as:

pPB0266: ApACCase::ApPGK1-1p-neoR(s)-ApPGK1:ApSAD2v1p-Ig-FADd8-ApSAD2v1:ApAMT1p::ApACCase

pPB0267: ApACCase::ApPGK1-1p-neoR(s)-ApPGK1:ApSAD2v1p-Ps-FADd8-ApSAD2v1:ApAMT1p::ApACCase

Both pPB0266 and pPB0267 have the same vector backbone, target genomic locus, selectable marker cassette, 3′UTR, and relevant restriction sites like pPB0265 differing only in the enzyme tested and the promoter being used to drive it. Plasmid pPB0266 contains the ApSAD2v1 promoter driving I. galbana FADdelta-8 while pPB0267 contains ApSAD2v1 driving P. salina FADdelta-8.

The sequence of the ApSAD2v1-Ig-FADdelta-8-ApSAD2v1 3UTR in pPB0266 is depicted in FIG. 13.

SpeI and XbaI restriction sites, at the beginning and end of the cassette, are depicted in lowercase bold. A. protothecoides stearoyl ACP desaturase 2 (ApSAD2v1) promoter (indicated as small case boxed text) drives the expression of codon-optimized I. galbana FADdelta-8 (Ig-FADdelta-8) gene. The initiator ATG and terminator TGA for Ig-FADdelta-8 are indicated in uppercase italics, while the coding region is indicated in lowercase italics. The A. protothecoides stearoyl ACP desaturase terminator (ApSAD2v1 terminator) region is indicated in small capitals. The final construct was sequenced to ensure correct reading frames and targeting sequences.

The sequence of the ApSAD2v1-Ps-FADdelta-8-ApSAD2v1 3UTR cassette in pPB0267 is depicted in FIG. 14.

SpeI and XbaI restriction sites, at the beginning and end of the cassette, are depicted in lowercase bold. A. protothecoides stearoyl ACP desaturase 2 (ApSAD2v1) promoter (indicated as small case boxed text) drives the expression of codon-optimized P. salina FADdelta-8 (Ps-FADdelta-8) gene. The initiator ATG and terminator TGA for Ps-FADdelta-8 are indicated in uppercase italics, while the coding region is indicated in lowercase italics. The A. protothecoides stearoyl ACP desaturase terminator (ApSAD2v1 terminator) region is indicated by small capitals. The final construct was sequenced to ensure correct reading frames and targeting sequences.

pPB0265, pPB0266, and pPB0267 were transformed into Phycoil strains PES-04, PES-05, or PES-07, and primary transformants were selected on sucrose-containing growth media without thiamine and supplemented with the antibiotic G418. Single clonally purified colonies were grown under standard lipid production conditions in shake flasks. The resulting profiles from a set of representative clones arising from transformations with pPB0265, pPB0266, and pPB0267 constructs are shown in Tables 4, 5, and 6.

TABLE 4
Fatty acid profiles as a percentage of total fatty acids for parental strains (PES-04,
PES-05, and PES-07) and representative transformants containing Phycoil plasmids
pPB0265 (PES-04; 265-1; PES-04 265-2).

| Sample | C16:0 | C18:0 | C18:1n-9 | C18:2n-6 | C18:3n-3 | C20:2n-6 | C20:3n-6 |
|---|---|---|---|---|---|---|---|
| name | Palmitic | Stearic | Oleic | Linoleic | ALA | EDA | DGLA |
| PES-05 | 11.32 | 3.88 | 49.89 | 23.9 | 0.99 | 8.98 | — |
| Parent | | | | | | | |
| PES-07 | 10.93 | 4.12 | 52.50 | 14.26 | 1.03 | 16.8 | — |
| Parent | | | | | | | |
| PES-04 | 11.88 | 2.58 | 62.65 | 8.97 | 1.13 | 4.76 | 3.42 |
| Parent | | | | | | | |
| PES-04; | 12.43 | 3.43 | 50.75 | 14.44 | 0.98 | 12.93 | 4.33 |
| 265-1 | | | | | | | |
| PES-04; | 12.49 | 3.18 | 49.81 | 14.89 | 1.00 | 13.40 | 4.45 |
| 265-2 | | | | | | | |

Having identified At-LPCAT1 and Ig-FADdelta-8 or Ps-FADdelta-8, besides Ig-FAEdelta9, as key enzyme activities required for the accumulation of EDA and DGLA in our organism, albeit in different strains, this series of experiments aimed to combine the acyltransferase activity of At-LPCAT1 and delta-8 fatty acid desaturase activity of IgFADdelta8 or Ps-FADdelta-8 and create a single strain with boosted EDA and DGLA. Transformation of pPB0265 expressing At-LPCAT1 in Phycoil PES-04 (elongase-FAD8 desaturase) strain resulted in a significant reduction of C18:1 n-9 from 62.25% in parent PES-04 to 49-51% in representative lines PES-04; 265-1 and PES-04; 265-2 (Table 4). Reduction in C18:1n-9 was concomitant with a significant increase in C18:2n6 from ˜9% in parent PES-04 to ˜15% in PES-04; 265-1 (14.44%) and PES-04; 265-2 (14.89%) again demonstrating the pivotal role of At-LPCAT1 in increasing channeling of substrate C18:1 n-9 to phospholipids where they get desaturated into C18:2n-6 by endogenous FAD2 enzyme. Since PES-04 already expresses heterologous Ig-ASE2 delta9-FAE and Ps-FADdelta-8 enzymes, increased C18:2n-6 was available for elongation and desaturation to EDA and DGLA, respectively. As expected, EDA levels increased from 4.76% in the parent PES-04 to 12.93% in PES-04; 265-1, and 13.40% in PES-04; 265-2. There was also a subtle increase in DGLA from 3.42% in PES-4 to 4.33% in PES-04; 265-1 and 4.45% in PES-04; 265-2. Since the construct was designed to hijack the promoter of endogenous ACCase with ApAMT1 promoter, we surmise that this subtle increase in DGLA is due to increased availability of malonyl Co-A in the cytosol resulting in increased EDA which then gets converted into DGLA by Ps-FADdelta-8.

pPB0266 (containing Ig-FADdelta-8) and pPB0267 (containing Ps-FADdelta-8) were transformed into Phycoil strain PES-05 (expressing IgASE2 delta9-FAE and At-PDCT). As demonstrated earlier, At-PDCT seems to negatively affect the production of LCPUFA in our organism since C18:2n-6 liberated from phospholipids by At-PDCT activity gets tied into DAGs thereby becoming unavailable for elongation to EDA. Nevertheless, the expression of either Ig-FADdelta-8 or Ps-FADdelta-8 enzymes in PES-05 led to a subtle increase in elongation of C18:2n-6 to C20:2n-6 (measured by reduction in C18:2n-6 from 23.9% in PES-05 to 21.27% in PES-05; 266-1 and 21.49% in PES-05; 267-1) and appearance of DGLA (1.96% and 3.36% in PES-05; 266-1 and PES-05; 267-1 respectively, Table 5). There was an increase in C18:1 n-9 content in both PES-05; 266-1 (54.44%) and PES-05; 267-1 (53.02%) over the parent PES-05 (49.89%) most likely because of the increased endogenous ketoacyl synthase (KAS) and stearoyl ACP desaturase (SAD) activity due to enhanced channeling of C18:2n-6 out of the phospholipid membranes.

TABLE 5
Fatty acid profiles as a percentage of total fatty acids for parental strains (PES-04,
PES-05, and PES-07) and representative derivative transformants containing Phycoil
plasmids pPB0266 (PES-05; 266-1), or pPB0267 (line PES-01; 267-1).

| | C16:0 | C18:0 | C18:1n-9 | C18:2n-6 | C18:3n-3 | C20:2n-6 | C20:3n-6 |
|---|---|---|---|---|---|---|---|
| Sample name | Palmitic | Stearic | Oleic | Linoleic | ALA | EDA | DGLA |
| PES-04 | 11.88 | 2.58 | 62.65 | 8.97 | 1.13 | 4.76 | 3.42 |
| Parent | | | | | | | |
| PES-07 | 10.93 | 4.12 | 52.5 | 14.26 | 1.03 | 16.8 | — |
| Parent | | | | | | | |
| PES-05 | 11.32 | 3.88 | 49.89 | 23.9 | 0.99 | 8.98 | — |
| Parent | | | | | | | |
| PES-05; 266-1 | 9.96 | 2.51 | 54.44 | 21.27 | 0.95 | 8.93 | 1.96 |
| PES-05; 267-1 | 10.85 | 3.20 | 53.02 | 21.49 | 0.74 | 6.86 | 3.36 |

Constructs pPB0266 (containing Ig-FADdelta-8) and pPB0267 (containing Ps-FADdelta-8) were also transformed into Phycoil strain PES-07 (expressing IgASE2 delta9-FAE and At-LPCAT1). Representative lines from both pPB0266 and pPB0267 transformed into PES-07 showed a diminution of C18:2n-6, with the appearance of DGLA (because of introduced FADdelta-8 enzymes) in our samples (table 6). PES-07 lines expressing Ig-FADdelta-8 (PES-07; 266-1, PES-07; 266-2, PES-07; 266-3) produced less DGLA compared to lines expressing Ps-FADdelta-8 (PES07; 267-1, PES07; 267-2, PES-07; 267-3, PES-07; 267-7) plausibly pointing towards a better desaturase activity of Ps-FADdelta-8 in our organism. As observed before for PES-04; 265-1 and PES-04; 265-2, there was a subtle but consistent increase in DGLA production in PES-07; 266-1, 266-2, 266-3, and 266-7 strains compared with PES-04 strain that expresses the same Ps-FADdelta-8 enzyme. This increase most likely stems from the increased elongation of C18:2n6 to C20:2n-6 because of increased Malonyl Co-A due to the hijacking of ACCase endogenous promoter.

TABLE 6
Fatty acid profiles as a percentage of total fatty acids for parental strain (PES-04,
PES-05, and PES-07) and representative transformants containing Phycoil plasmids
pPB0265 (PES-07; 266-1; PES-07; 266-2, PES-07; 266-3, PES-07; 266-4), and pPB0267
(lines PES-07; 267-1; PES-07; 267-2, PES-07; 267-3, PES-07; 267-4, PES-07; 267-7).

| | C16:0 | C18:0 | C18:1n-9 | C18:2n-6 | C18:3n-3 | C20:2n-6 | C20:3n-6 |
|---|---|---|---|---|---|---|---|
| Sample name | Palmitic | Stearic | Oleic | Linoleic | ALA | EDA | DGLA |
| PES-04 | 11.88 | 2.58 | 62.65 | 8.97 | 1.13 | 4.76 | 3.42 |
| Parent | | | | | | | |
| PES-05 | 11.32 | 3.88 | 49.89 | 23.90 | 0.99 | 8.98 | — |
| Parent | | | | | | | |
| PES-07 | 10.93 | 4.12 | 52.50 | 14.26 | 1.03 | 16.80 | — |
| Parent | | | | | | | |
| PES-07; 266-1 | 10.79 | 4.03 | 56.26 | 10.12 | 0.82 | 15.62 | 1.66 |
| PES-07; 266-2 | 11.11 | 3.71 | 56.58 | 10.27 | 0.77 | 15.34 | 1.88 |
| PES-07; 266-3 | 11.19 | 4.06 | 54.21 | 10.67 | 0.93 | 16.51 | 2.06 |
| PES07; 267-1 | 10.6 | 3.91 | 56.45 | 10.12 | 0.87 | 13.06 | 4.64 |
| PES07; 267-2 | 10.99 | 4.09 | 56.26 | 10.08 | 1.15 | 13.02 | 3.98 |
| PES-07; 267-3 | 11.25 | 3.6 | 55.48 | 9.93 | 0.76 | 14.20 | 4.45 |
| PES-07; 267-7 | 10.46 | 3.89 | 53.88 | 9.69 | 0.7 | 16.42 | 4.22 |

### Example 5. Expression of Heterologous Fatty Acid Desaturase 5 Kickstarts ARA Production in Phycoil Strains Producing DGLA

Having demonstrated that our organism can support the production of EDA and DGLA, we next explored if a portion of the DGLA can be further desaturated to make arachidonic acid (ARA). To explore this possibility, candidate fatty acid desaturase 5 (FADdelta-5) genes from Euglena gracilis (Eg-FADdelta-5; Accession No: CBH30563), Phaeodactylum tricornutum (Pt-FADdelta-5; Accession No: AAL92562), Thalassiosira pseudonana CCMP1335 (Tp-FADdelta-5; Accession No: XP_002296867), Mortierella alpina (Ma-FADdelta-5; Accession No: AF054824), and Oblongichytrium sp. SEK 347 (Oblongi-FADdelta-5; Accession No: BAG71007) were codon-optimized and synthesized for expression in engineered Phycoil strains PES-04 and PES-07. Several constructs detailed below were made to test the functionality of the FADdelta-5 enzymes in the engineered strains. We also expressed a second copy of Ps-FADdelta-8 in these constructs to determine if that would result in increased DGLA substrate ready for conversion to ARA by any candidate FADdelta-5 enzymes. Constructs pPB0274, pPB0275, pPB0276, pPB0303, and pPB0304 designed for transformation into PES-04 and PES-07 can be written as below.

pPB0274 ApACCase::ApPGK1-1p-neoR(s)-ApPGK13′UTR:ApSAD2v1p-PsFADd8-ApSAD2v13′UTR:ApAMT2v1p-Eg-FADd5-ApPGH3′UTR:ApAMT1p::ApACCase

pPB0275 ApACCase::ApPGK1-1p-neoR(s)-ApPGK13′UTR:ApSAD2v1p-PsFADd8-ApSAD2v13′UTR:ApAMT2v1p-Pt-FADd5-ApPGH3′UTR:ApAMT1p::ApACCase

pPB0276 ApACCase::ApPGK1-1p-neoR(s)-ApPGK13′UTR:ApSAD2v1p-PsFADd8-ApSAD2v13′UTR:ApAMT2v1p-Tp-FADd5-ApPGH3′UTR:ApAMT1p::ApACCase

pPB0303 ApACCase::ApPGK1-1p-neoR(s)-ApPGK13′UTR:ApSAD2v1p-PsFADd8-ApSAD2v13′UTR:ApAMT2v1p-Ma-FADd5-ApPGH3′UTR:ApAMT1p::ApACCase

pPB0305 ApACCase::ApPGK1-1p-neoR(s)-ApPGK13′UTR:ApSAD2v1p-PsFADd8-ApSAD2v13′UTR:ApAMT2v1p-Oblongi-FADd5-ApPGH3′UTR:ApAMT1p::ApACCase

The sequence of the transforming DNA construct pPB0274 is shown below in FIG. 15. Relevant restriction sites in the construct are indicated in lowercase, bold, and are from 5′-3′ HindIII, KpnI, SpeI, XbaI, AfIII, and HindIII, respectively. HindIII sites delimit the 5′ and 3′ ends of the transforming DNA. Underlined, uppercase sequences represent genomic DNA from A. protothecoides PB5 that permit targeted integration of heterologous gene cassettes and ApAMT1 promoter at the ACCase locus via homologous recombination. Proceeding from 5′ to 3′, the selection cassette contains the A. protothecoides phosphoglycerate kinase 1 (ApPGK1) promoter in lowercase, boxed text, driving expression of neomycin phosphotransferase II gene (Neo, codon-optimized for expression in A. protothecoides and encoding neomycin phosphotransferase II, thereby enabling the strain to grow on aminoglycoside antibiotic G418). The initiator ATG and terminator TGA for Neo are indicated in uppercase italics while the rest of the sequence is indicated in lowercase italics. The terminator region of the A. protothecoides phosphoglycerate kinase 1 (ApPGK1 terminator) is indicated by small capitals followed by A. protothecoides stearoyl ACP desaturase ApSAD2v1 promoter (indicated as small case boxed text) driving the expression of codon-optimized P. salina FADdelta-8 (Ps-FADdelta-8) gene. The initiator ATG and terminator TGA for Ps-FADdelta-8 are indicated by uppercase italics, while the coding region is indicated in lowercase italics. The A. protothecoides stearoyl ACP desaturase terminator (ApSAD2v1 terminator) region is indicated by small capitals followed by A. protothecoides ammonium transporter 2 (ApAMT2v1) promoter (indicated as small case boxed text) driving the expression of codon-optimized E. gracilis FADdelta-5. The initiator ATG and terminator TGA for Eg-FADdelta-5 are indicated in uppercase italics, while the coding region is indicated in lowercase italics. The ApPGH terminator region is indicated by small capitals followed by A. protothecoides ammonium transporter 1 (ApAMT1) promoter. Immediately following the ApAMT1 promoter is the ApACCase genomic region indicated by underlined uppercase text with the ATG initiator codon of the ACCase gene in bold letters. The final construct was sequenced to ensure correct reading frames and targeting sequences.

Constructs pPB0275, pPB0276, pPB0303, and pPB0305 have the same vector backbone; selectable marker, promoters, and 3′ UTR as pPB0274, differing only in the respective FADdelta-5 genes being screened. Relevant restriction sites in these constructs are also the same as in pPB0177. FIGS. 16-19 indicate the sequence of Pt-FADdelta-5, Tp-FADdelta-5, Ma-FADdelta-5, and Oblongi-FADdelta-5 respectively in lowercase with the initiator ATG and terminator TGA codons in uppercase italics.

pPB0274, pPB0275, pPB0276, pPB0303, and pPB0305 were transformed into Phycoil strain PES-04 (expressing Ig-delta-9FAE and Ps-FADdelta-8) or PES-07 (expressing Ig-delta-9FAE and At-LPCAT1) and primary transformants were selected on sucrose-containing growth media without thiamine and supplemented with the antibiotic G418. Single clonally purified colonies were grown under standard lipid production conditions in shake flasks. The resulting profiles from a set of representative derivative clones arising from transformations with pPB0274, pPB0275, and pPB0276, pPB0303, and pPB0305 constructs are shown in Tables 7 and 8.

PES-04 transformed with pPB0274, pPB0276, pPB0303, and pPB0305 (data not shown) showed the fatty acid profile like the parent PES-04 with no additional peak whatsoever (Table 7). The second copy of Ps-FADdelta-8 did not seem to drastically affect the DGLA production in derivative transgenic lines even though there were several lines (e.g., PES-04; 274-2; PES-04; 274-3, PES-04; 276-2) that showed elevated DGLA levels never seen before in any of our previous strains (cf. Table 6, example 4 above). Since the constructs were again targeted to the ACCase locus attempting to upregulate the downstream ACCase gene, it is quite plausible that the increased DGLA seen in these strains is due to boosted elongation because of increased malonyl Co-A as discussed in earlier examples.

For PES-04 lines transformed with pPB0275 DNA, containing Phaeodactylum tricornutum FADdelta-5 (Pt-FADdelta-5), a specific peak corresponding to arachidonic acid (ARA) was observed. Derivative transgenic line PES-04; 275-5 produced the highest level of ARA up to 1.31% followed by lines PES-04; 275-2 and PES-04; 275-1 with 1.19% and 1.13% ARA. The appearance of ARA in these lines was concomitant with the decrease in DGLA levels suggesting that the newly introduced Pt-FADdelta-5 uses the DGLA as the substrate and desaturates it to produce ARA.

TABLE 7
Fatty acid profiles as a percentage of total fatty acids for parental strain (PES-04)
and representative derivative transgenic lines transformed with Phycoil plasmids pPB0274
(PES-04; 274-2, PES-04; 274-3, PES-04; 274-4), pPB0275 (PES-04; 275-1, PES-04;
275-2, PES-04; 275-5, PES-04; 275-14), pPB0276 (PES-04;276-2, PES-04;276-4), and
pPB0303 (PES-04; 303-1). No transformants were recovered for PES-04 transformed with
pPB0305.

| Sample | C16:0 | C18:0 | C18:1n-9 | C18:2n-6 | C18:3n-3 | C20:2n-6 | C20:3n-6 | C20:4n-6 |
|---|---|---|---|---|---|---|---|---|
| name | Palmitic | Stearic | Oleic | Linoleic | ALA | EDA | DGLA | ARA |
| PES-04 | 11.88 | 2.58 | 62.65 | 8.97 | 1.13 | 4.76 | 3.42 | — |
| Parent | | | | | | | | |
| PES-04; | 10.76 | 2.63 | 68.89 | 7.23 | 1.18 | 2.95 | 5.50 | — |
| 274-2 | | | | | | | | |
| PES-04; | 10.85 | 3.04 | 68.09 | 6.93 | 1.03 | 3.18 | 5.61 | — |
| 274-3 | | | | | | | | |
| PES-04; | 11.97 | 2.80 | 63.07 | 6.95 | 0.63 | 9.26 | 4.00 | — |
| 274-4 | | | | | | | | |
| PES-04; | 12.50 | 2.73 | 64.95 | 7.72 | 0.66 | 6.10 | 3.26 | 1.13 |
| 275-1 | | | | | | | | |
| PES-04; | 12.60 | 3.17 | 63.23 | 7.87 | 0.72 | 6.83 | 3.32 | 1.19 |
| 275-2 | | | | | | | | |
| PES-04; | 12.65 | 3.27 | 62.76 | 7.84 | 0.67 | 7.06 | 3.41 | 1.31 |
| 275-5 | | | | | | | | |
| PES-04; | 12.70 | 1.62 | 64.81 | 7.85 | 0.75 | 8.19 | 2.04 | 0.85 |
| 275-14 | | | | | | | | |
| PES-04; | 11.64 | 3.18 | 65.76 | 8.10 | 1.04 | 3.99 | 5.37 | — |
| 276-2 | | | | | | | | |
| PES-04; | 12.05 | 3.02 | 63.15 | 7.12 | 0.55 | 9.32 | 3.64 | — |
| 276-4 | | | | | | | | |
| PES-04; | 10.61 | 4.28 | 68.89 | 5.86 | 0.61 | 6.73 | 2.15 | — |
| 303-1 | | | | | | | | |

We also transformed the constructs pPB0274, pPB0275, pPB0276, pPB0303 and pPB0305 into Phycoil strain PES-07 (expressing Ig-delta-9FAE and At-LPCAT1). Since PES-07 lacks a FADdelta-8 enzyme, the introduction of Ps-FADdelta-8 via the above constructs resulted in DGLA production in all derivative strains (Table 8). The amount of DGLA produced was comparable to that seen in the derivative transgenic lines obtained in the PES-04 parent background described above (Table 7).

TABLE 8
Fatty acid profiles as a percentage of total fatty acids for parental strain (PES-07)
and representative derivative transgenic lines transformed with Phycoil plasmids pPB0274
(line PES-07; 274-3), pPB0275 (lines PES-07; 275-1, PES-07; 275-3, PES-07; 275-5, PES-
07; 275-6), pPB0276 (lines PES-07; 276-1; PES-07; 276-2, PES-07; 276-3, and PES-07;
276-4), pPB0303 (lines PES-07; 303-2, PES-07; 303-10, PES-07; 303-11), pPB0305 (lines
PES-07; 305-11, PES-07; 305-12).

| Sample | C16:0 | C18:0 | C18:1n-9 | C18:2n-6 | C18:3n-3 | C20:2n-6 | C20:3n-6 | C20:4n-6 |
|---|---|---|---|---|---|---|---|---|
| name | Palmitic | Stearic | Oleic | Linoleic | ALA | EDA | DGLA | ARA |
| PES-07 | 10.93 | 4.12 | 52.5 | 14.26 | 1.03 | 16.80 | — | — |
| Parent | | | | | | | | |
| PES-07; | 11.42 | 2.99 | 54.54 | 11.2 | 1.37 | 14.36 | 4.13 | — |
| 274-3 | | | | | | | | |
| PES-07; | 11.14 | 3.91 | 56.84 | 7.45 | 0.7 | 17.38 | 1.58 | 0.55 |
| 275-1 | | | | | | | | |
| PES-07; | 11.19 | 3.86 | 56.32 | 7.97 | 0.71 | 17.33 | 1.59 | 0.62 |
| 275-3 | | | | | | | | |
| PES-07; | 11.2 | 3.94 | 57.32 | 7.48 | 0.71 | 17.05 | 1.43 | 0.46 |
| 275-5 | | | | | | | | |
| PES-07; | 11.86 | 3.42 | 54.76 | 7.55 | 0.69 | 19.39 | 1.36 | 0.51 |
| 275-6 | | | | | | | | |
| PES-07; | 10.09 | 3.24 | 56.43 | 9.72 | 0.89 | 14.07 | 5.57 | — |
| 276-1 | | | | | | | | |
| PES-07; | 11.22 | 4.39 | 53.68 | 10.29 | 0.76 | 15.38 | 3.97 | — |
| 276-2 | | | | | | | | |
| PES-07; | 11.53 | 4.01 | 53.15 | 10.44 | 0.82 | 15.73 | 4.32 | — |
| 276-3 | | | | | | | | |
| PES-07; | 11.85 | 3.93 | 52.63 | 9.87 | 0.84 | 16.93 | 3.56 | — |
| 276-4 | | | | | | | | |
| PES-07; | 11.80 | 3.57 | 52.97 | 8.39 | 0.98 | 17.94 | 3.71 | — |
| 303-2 | | | | | | | | |
| PES-07; | 11.38 | 3.45 | 53.83 | 10.53 | 1.03 | 16.40 | 3.27 | — |
| 303-10 | | | | | | | | |
| PES-07; | 12.05 | 3.33 | 52.02 | 8.70 | 1.04 | 17.59 | 4.30 | — |
| 303-11 | | | | | | | | |
| PES-07; | 11.38 | 3.45 | 53.84 | 10.54 | 1.04 | 16.48 | 3.27 | — |
| 305-12 | | | | | | | | |

As observed for derivative transgenic lines in the PES-07 background above (Table 7), only pPB0275 transformed into PES-04 resulted in ARA peaks concomitant with the decrease in the substrate DGLA levels further demonstrating that the combination of Ig-delta-9FAE, PS-FADdela-08, and Pt-FADdelta-5 enzymes can kickstart production of LC-PUFAs including EDA, DGLA, and ARA in Phycoil host A. protothecoides PB5.

### Example 6: Increasing at-LPCAT1 Activity by Modulating its Expression Doubles the Production of EDA in Phycoil Engineered Strains

Expression of At-LPCAT1 significantly increases the channeling of C18:1n-9 into phospholipids where it is converted into C18:2n-6 which is then either incorporated into DAGs by endogenous choline-phosphotransferase activity or elongated to EDA by heterologous Ig-delta-9FAE. At-LPCAT1 consistently resulted in a two- or more-fold increase in EDA levels in all engineered strains expressing Ig-delta-9FAE (examples 3, 4, and 5). Even after optimizing its incorporation into phospholipids and further desaturation into C18:2, and elongation to EDA, there is still a significant amount of C18:1 n-9 (˜50-54%) potentially available for phospholipid channeling and downstream modification. We surmised that increasing the At-LPCAT1 activity would further boost EDA levels in our engineered strains. To test this hypothesis, we transformed a construct (pPB0304) expressing At-LPCAT1 and Oblongichytrium sp. SEK 347 FADdelta-5 (Oblongi-FADdelta-5) into Phycoil strain PES-07 (already expressing a single copy of At-LPCAT1 besides Ig-delta-9FAE). PES-07 does not express any heterologous FADdelta-8 enzymes and thus produces no DGLA that could be used as a substrate by Oblongi-FADdelta-5 to produce ARA. Besides, from our earlier experiments, expression of Oblongi-FADdelta-5 (example 5, pPB0305 transformed into PES-04 or PES-07) did not result in any ARA suggesting that this enzyme is not effective at desaturating DGLA to ARA in our host. Thus, the construct pPB0305 afforded us a good opportunity to test the effect of At-LPCAT1 copy number on EDA levels.

Construct pPB0304 can be written as:

pPB0304 ApACCase::ApPGK1-1p-neoR(s)-ApPGK13′UTR:ApAMT2v1p-ApLPCAT1-ApSAD2v13′UTR:ApSAD2v1p-Oblongi-FADd5-ApPGH3′UTR:ApAMT1p::ApACCase

The sequence of the transforming DNA construct pPB0304 is shown below in FIG. 20. Relevant restriction sites in the construct are indicated in lowercase, bold, and are from 5′-3′ HindIII, KpnI, SpeI, XbaI, AfIII, and HindIII, respectively. HindIII sites delimit the 5′ and 3′ ends of the transforming DNA. Underlined, uppercase sequences represent genomic DNA from A. protothecoides PB5 that permit targeted integration of heterologous gene cassettes and ApAMT1 promoter at the ACCase locus via homologous recombination. Proceeding from 5′ to 3′, the selection cassette contains the A. protothecoides phosphoglycerate kinase 1 (ApPGK1) promoter in lowercase, boxed text, driving expression of neomycin phosphotransferase II gene (Neo, codon-optimized for expression in A. protothecoides and encoding neomycin phosphotransferase II, thereby enabling the strain to grow on aminoglycoside antibiotic G418). The initiator ATG and terminator TGA for Neo are indicated in uppercase italics while the rest of the sequence is indicated in lowercase italics. The terminator region of the A. protothecoides phosphoglycerate kinase 1 (ApPGK1 terminator) is indicated by small capitals followed by A. protothecoides ammonium transporter 2 (ApAMT2v1) promoter (indicated as small case boxed text) driving the expression of codon-optimized At-LPCAT1 gene. The initiator ATG and terminator TGA for At-LPCAT1 are indicated in uppercase italics, while the coding region is indicated in lowercase italics. The A. protothecoides stearoyl ACP desaturase terminator (Ap-SAD2v1 terminator) region is indicated by small capitals followed by A. protothecoides stearoyl ACP desaturase ApSAD2v1 promoter (indicated as small case boxed text) driving the expression of codon-optimized Oblogichytrium sp. SEK 347 FADdelta-5. The initiator ATG and terminator TGA for Oblongi-FADdelta-5 are indicated in uppercase italics, while the coding region is indicated in lowercase italics. The ApPGH terminator region is indicated by small capitals followed by A. protothecoides ammonium transporter 1 (ApAMT1) promoter. Immediately following the ApAMT1 promoter is the ApACCase genomic region indicated by underlined uppercase text with the ATG initiator codon of the ACCase gene in bold letters. The final construct was sequenced to ensure correct reading frames and targeting sequences.

pPB0304 was transformed into Phycoil strain PES-07 (expressing Ig-delta-9FAE and At-LPCAT1) and primary transformants were selected on sucrose-containing growth media without thiamine and supplemented with the antibiotic G418. Single clonally purified colonies were grown under standard lipid production conditions in shake flasks. The resulting profiles from a set of representative derivative clones arising from transformations with the construct pPB0304 are shown in Table 9.

TABLE 9
Fatty acid profiles as a percentage of total fatty acids for parental strain (PES-07)
and representative derivative transgenic lines transformed with Phycoil plasmid pPB0304
(line PES-07; 304-1, PES-07; 304-3, PES-07; 304-4)

| Sample | C16:0 | C18:0 | C18:1n-9 | C18:2n-6 | C18:3n-3 | C20:2n-6 | C20:3n-6 | C20:4n-6 |
|---|---|---|---|---|---|---|---|---|
| name | Palmitic | Stearic | Oleic | Linoleic | ALA | EDA | DGLA | ARA |
| PES-07 | 10.93 | 4.12 | 52.5 | 14.26 | 1.03 | 16.80 | — | — |
| Parent | | | | | | | | |
| PES-07; | 12.48 | 1.01 | 39.33 | 19.91 | 2.09 | 25.16 | — | — |
| 304-1 | | | | | | | | |
| PES-07; | 12.19 | 1.10 | 36.7 | 17.99 | 2.37 | 29.66 | — | — |
| 304-3 | | | | | | | | |
| PES-07; | 12.28 | 1.13 | 36.33 | 18.39 | 2.41 | 30.60 | — | — |
| 304-4 | | | | | | | | |

The expression of the second copy of At-LPCAT1 at the upregulated ACCase locus resulted in nearly doubling of the EDA from the parent strain. Transgenic lines PES-07; 304-1, PES-07; 304-3, and PES-07; 304-4 produced 25.16%, 29.66%, and 30.60% EDA, respectively, compared to parent PES-07 which produce ˜ 17% EDA. Given that we still have between 36-39% of available C18:1 n-9, configurations further boosting the LPCAT activity in our host will produce more EDA further boosting downstream elongation and desaturation to produce DGLA, ARA, and other essential LCPUFAs.

### Example 7: Construction of Phycoil Strains Producing EDA, DGLA, ARA, and EPA

Examples 1-6 described above helped us identify various enzymes and configurations that would result in the production of LCPUFAs in A. protothecoides PB5. However, it took us three consecutive transformations to reach ARA. In the next set of experiments, we combined the activities of these enzymes into as few constructs as possible in an effort to reach ARA and eventually EPA in just two or three successive transformations. To accomplish this, we first decided to simultaneously express Ig-delta-9FAE, At-LPCAT1, and Ps-FADdelta-8 enzymes in our wild-type A. protothecoides PB5 strain. We made a construct pPB0306 to accomplish this. In the pPB0306 construct, each heterologous enzyme was driven by a distinct promoter and termination signal (3′UTRs). ApSAD2v1 was used to drive the Ig-delta-9 FAE, ApAMT2v1 drove the At-LPCAT1, while ApAMT1 drove the expression of Ps-FADdelta-8. In terms of promoter strength, our past data suggests that ApSAD2v1 is stronger than the ApAMT1 promoter. To avoid uncontrolled gene amplification via recombination in our organism, we did not wish to use a single promoter (e.g., ApSAD2v1) to drive more than one enzyme at a given locus. Thus, in pPB0306, Ps-FADdelta-8 was driven by an ApAMT1 promoter, unlike the above-described examples where it was driven by a stronger ApSAD2v1 promoter. Hence, we expected that the Ps-FADdelta-8 expression and the resulting DGLA might not be optimal in our first round of transformations. The construct pPB0306 can be written as:

pPB0306: ApDAO1::CrTUB2-ScSUC2-ApPGH3′UTR:ApSAD2v1p-IgFAEd9(ASE2)elongase-ApSAD2v13′UTR:ApAMT2v1-AtLPCAT1ApPGK13′UTR:ApAMT1p-PsFADd8-ApHsp903′UTR::ApDAO1

The sequence of the transforming DNA construct pPB0306 is shown below in FIG. 21.

Relevant restriction sites in the construct are indicated in lowercase bold and are from 5′-3′ EcoRV, SpeI, NotI, AfIII, XbaI, and EcoRV, respectively. EcoRV sites delimit the 5′ and 3′ ends of the transforming DNA. Underlined, uppercase sequences represent genomic DNA from A. protothecoides PB5 that enable targeted integration of transforming DNA containing various heterologous gene cassettes at the D-aspartate oxidase 1 (DAO1) locus via homologous recombination. Proceeding from 5′ to 3′, the selection cassette contains the C. reinhardtii beta-tubulin 2 (CrTUB2) promoter in lowercase, boxed text, driving expression of Saccharomyces cerevisiae SUC2 gene (ScSUC2, codon-optimized for expression in A. protothecoides and encoding sucrose invertase, thereby enabling the strain to utilize exogenous sucrose). The initiator ATG and terminator TGA for ScSUC2 are indicated by uppercase italics, while the coding region is indicated in lowercase italics. The terminator region of the A. protothecoides enolase gene (ApPGH) gene is indicated by small capitals followed by the A. protothecoides stearoyl ACP desaturase (ApSAD2v1) promoter (indicated as small case boxed text) driving the expression of codon-optimized Ig-delta-9FAE gene. The initiator ATG and terminator TGA for Ig-delta-9FAE are indicated by uppercase italics, while the coding region is indicated in lowercase italics. The A. protothecoides stearoyl ACP desaturase terminator (ApSAD2v1 terminator) region is indicated by small capitals followed by A. protothecoides A. protothecoides ammonium transporter 2 (ApAMT2v1) promoter (indicated as small case boxed text) driving the expression of codon-optimized At-LPCAT1. The initiator ATG and terminator TGA for At-LPCAT1 are indicated by uppercase italics, while the coding region is indicated in lowercase italics. The ApPGK1 terminator region is indicated by small capitals followed by A. protothecoides ammonium transporter 1 (ApAMT1) promoter driving the expression of Ps-FADdelta-8. The initiator ATG and terminator TGA for Ps-FADdelta-8 are indicated by uppercase italics, while the coding region is indicated in lowercase italics. The terminator region of the A. protothecoides heat shock protein 90 (ApHSP90) gene is indicated by small capitals followed by the A. protothecoides PB5 D-aspartate oxidase 1 (DAO1) genomic region indicated by the underlined uppercase text. The final construct was sequenced to ensure correct reading frames and targeting sequences.

pPB0306 was transformed into wildtype A. protothecoides PB5. Primary transformants were selected on sucrose-containing growth media. Single clonally purified colonies were grown under standard lipid production conditions in shake flasks. The resulting profiles from a set of representative clones arising from transformations with the pPB0306 construct are shown in Table 10.

TABLE 10
Fatty acid profiles as a percentage of total fatty acids for parental strain (PB5)
and representative derivative transgenic lines transformed with Phycoil plasmid
pPB0306 (lines PB5; 306-3, PB5; 306-5, PB5; 306-6, PB5; 306-20).

| Sample | C16:0 | C18:0 | C18:1n-9 | C18:2n-6 | C18:3n-3 | C20:2n-6 | C20:3n-6 |
|---|---|---|---|---|---|---|---|
| name | Palmitic | Stearic | Oleic | Linoleic | ALA | EDA | DGLA |
| PB5 | 12.07 | 2.93 | 69.17 | 13.86 | 1.97 | — | — |
| PB5; 306-3 | 12.40 | 2.19 | 45.17 | 14.31 | 1.41 | 19.00 | 1.86 |
| PB5; 306-5 | 11.95 | 2.84 | 47.00 | 14.27 | 1.34 | 18.10 | 1.77 |
| PB5; 306-6 | 12.02 | 2.43 | 50.19 | 15.15 | 1.54 | 14.85 | 1.57 |
| PB5; 306-20 | 12.68 | 2.13 | 42.01 | 10.76 | 1.69 | 25.16 | 2.51 |

Expressing Ig-delta-9FAE, At-LPCAT1, and Ps-FADdelta-8 from the same transforming DNA targeted to the DAO1 genomic locus resulted in the similar fatty acid profiles that we had gotten by expressing these three enzymes independently via successive transformations in earlier examples (1, 2 and 3 described above). There was a noticeable increase in the EDA levels not seen before with single copy expression of Ig-delta-9FAE and At-LPCAT1 [c.f. PES-01; 234-1 (aka PES-07); EDA=17.34, example 3 above]. The highest level of EDA seen was 25.16% in PB5; 306-20, followed by 19% in PB5; 306-3. This data points towards more optimal expression and/or activity of At-LPCAT1 and/or Ig-delta-9FAE when expressed together at the DAO1 locus. As expected, and explained above, the DGLA production took a bit of a hit because of the ApAMT1 promoter driving Ps-FADdelta-8, and in the best possible scenario we got 2.51% of DGLA in PB5; 306-20 [cf. PES-01; 239-1 (aka PES-04); DGLA=3.42% in example 2 above]. Nevertheless, we ended up with several transgenic lines that could be used to screen candidate FADdelta-17 enzymes in the presence of earlier identified Pt-FADdelta-5 (example 5 above) in an attempt to make EPA in our organism. PB5; 306-20 was banked as Phycoil engineered strain PES-08 and used as a parent strain for subsequent transformations.

Candidate fatty acid desaturase-17 (FADdelta-17) genes from Pythium aphanidermatum (Pa-FADdelta-17; Accession No: AOA52182), Phytophthora sojae (Pj-FADdelta-17; Accession No: FW362213) and Saprolegnia diclina (Sd-FADdelta-17; Accession No: Q6UB73) were codon-optimized and synthesized for expression in engineered Phycoil strains PES-08. Several constructs detailed below were made to test the functionality of the candidate FADdelta-17 enzymes in PES-08. Constructs pPB0333, pPB0334, and pPB0338 designed for transformation into PES-08 can be written as below.

pPB0333 ApACCase::ApSAD2v1-PtFADdelta05-ApPGH3′UTR:ApAMT2v1-PaFADdelta17-ApSAD2v13′UTR:ApHUP1-AtTHIC-ApHSP90:ApFATAv1::ApACCase pPB0334 ApACCase::ApSAD2v1-PtFADdelta05-ApPGH3′UTR:ApAMT2v1-Ps-FADdelta17-ApSAD2v13′UTR:ApHUP1-AtTHIC-ApHSP90:ApFATAv1::ApACCase pPB0338 ApACCase::ApAMT2v1-PtFADdelta05-ApPGH3′UTR:ApSAD2v1-SdFADdelta17-ApSAD2v13′UTR:ApHUP1-AtTHIC-ApHSP90:ApFATAv1::ApACCase The sequence of the transforming DNA construct pPB0333 is shown below in FIG. 22.

Relevant restriction sites in the construct are indicated in lowercase bold and are from 5′-3′ HindIII, EcoRV, NotI, AfIII, SpeI, and HindIII, respectively. HindIII sites delimit the 5′ and 3′ ends of the transforming DNA. Underlined uppercase sequences represent genomic DNA from A. protothecoides PB5 that permit targeted integration of heterologous gene cassettes and ApFATAv1 promoter at the ACCase locus via homologous recombination. Proceeding from 5′ to 3′, A. protothecoides stearoyl ACP desaturase (ApSAD2v1) promoter (indicated as small case boxed text) drives the expression of codon-optimized P. tricornutum FADdelta-5 (PtFADdelta-5) gene. The initiator ATG and terminator TGA for PtFADdelta-5 are indicated by uppercase italics, while the coding region is indicated in lowercase italics. The terminator region of the A. protothecoides enolase gene (ApPGH) gene is indicated by small capitals followed by A. protothecoides ammonium transporter 2 (ApAMT2v1) promoter (indicated as small case boxed text) driving the expression of codon-optimized P. aphanidermatum FADdelta-17 (Pa-FADdelta-17). The initiator ATG and terminator TGA for Pa-FADdelta-17 are indicated by uppercase italics, while the coding region is indicated in lowercase italics. The ApSAD2v1 terminator region is indicated by small capitals followed by HUP1 (hexose/H+ symporter) promoter (ApHUP1) driving the expression of the A. thaliana THIC gene (AtTHIC), codon-optimized for expression in A. protothecoides and encoding 4-amino-5-hydroxymethyl-2-methylpyrimidine synthase activity, thereby permitting the strain to grow in the absence of exogenous thiamine, is indicated by lowercase, boxed text. The initiator ATG and terminator TGA for AtTHIC are indicated by uppercase italics, while the coding region is indicated with lowercase italics. The terminator region of the A. protothecoides heat shock protein 90 (ApHSP90) gene is indicated by small capitals followed by the promoter from the A. protothecoides FATAv1 gene, encoding the acyl-ACP thioesterase, to replace the endogenous promoter of ACCase gene. Immediately following the ApFATAv1 promoter is the ApACCase genomic region indicated by underlined uppercase text with the ATG initiator codon of the ACCase gene in bold letters. The final construct was sequenced to ensure correct reading frames and targeting sequences.

pPB0334 construct has the same vector backbone, the genomic locus for integration, promoters, Pt-FADdelta5 enzyme, selectable marker cassette, and 3′ UTR's as pPB0333 differing only in the fatty acid desaturase delta-17 being tested. Instead of the Pa-FADdelta-17 gene, construct pPB0334 contains P. sojae FADdelta-17 (Ps-FADdelta-17) gene. Relevant restriction sites in the construct are also the same as in pPB0333. The sequence of Pj-FADdelta-5 contained in pPB0334 is shown in FIG. 23.

pPB0338 has the same vector backbone, the genomic locus for integration, Pt-FADdelta5 enzyme, and selectable marker cassette as pPB0333 and pPB0334. Relevant restriction sites in the construct are also the same as in pPB0333. However, it differs in the promoters used to drive Pt-FADdelta-5 and the fatty acid desaturase delta-17 being tested. In pPB0338, Pt-FADdelta-5 is driven by AMT2v1 promoter instead of ApSAD2v1 used in pPB333 and pPB0334. Also, the candidate S. diclina FADdelta-17 being tested in this construct is driven by the ApSAD2v1 promoter. The nucleotide sequence of ApAMT2v1-PtFADdelta-5-ApPGHUTR:ApSAD2v1-Sd-FADdelta-17-ApSAD2v1 UTR contained between EcoRV and AfIII restriction sites (depicted in bold lowercase letters) in pPB0338 is shown in FIG. 24.

The final construct was sequenced to ensure correct reading frames and targeting sequences.

pPB0333, pPB0334, and pPB0338 were transformed into Phycoil strain PES-08 (expressing Ig-delta-9FAE, AT-LPCAT1, and Ps-FADdelta-8) and primary transformants were selected on sucrose-containing growth media without thiamine. Single clonally purified colonies were grown under standard lipid production conditions in shake flasks. GC traces from representative derivative transgenic lines expressing FADdelta-17 enzymes arising from transformations of PES-08 with pPB0333 (PES-08; 333-7), pPB0334 (PES-08; 334-08), and pPB0338 (PES08-338-3 and PES-09; 338-10) are shown in FIG. 25. Analysis of GC traces revealed peaks corresponding to ARA and EPA over the control PES-08.

The resulting profiles from a set of representative clones arising from transformations with the pPB0333, pPB0334, and pPB0338 are shown in Table 11.

TABLE 11
Fatty acid profiles as a percentage of total fatty acids for parental strain (PES-08)
and representative derivative transgenic lines transformed with Phycoil plasmid pPB0333,
pPB0334, and pPB0338.

| Sample | C16:0 | C18:0 | C18:1n-9 | C18:2n-6 | C18:3n-3 | C20:2n-6 | C20:3n-6 | C20:4n-6 | C20:5n-3 |
|---|---|---|---|---|---|---|---|---|---|
| name | Palmitic | Stearic | Oleic | Linoleic | ALA | EDA | DGLA | ARA | EPA |
| PES-08 | 12.16 | 2.63 | 43.67 | 11.32 | 0.96 | 25.10 | 1.60 | — | — |
| Parent | | | | | | | | | |
| PES-08; | 12.57 | 2.84 | 51.70 | 7.66 | 0.88 | 20.17 | 0.88 | ND | ND |
| 333-7 | | | | | | | | | |
| PES-08; | 12.02 | 2.66 | 46.25 | 10.69 | 0.94 | 23.34 | 1.44 | ND | ND |
| 333-9 | | | | | | | | | |
| PES-08; | 12.47 | 2.29 | 52.64 | 7.24 | 0.87 | 20.63 | 0.79 | ND | ND |
| 334-6 | | | | | | | | | |
| PES-08; | 12.06 | 2.61 | 46.33 | 10.66 | 0.97 | 23.36 | 1.40 | ND | ND |
| 334-8 | | | | | | | | | |
| PES-08; | 11.44 | 2.68 | 49.07 | 6.11 | 0.60 | 25.93 | 0.43 | ND | ND |
| 334-11 | | | | | | | | | |
| PES-08; | 11.06 | 3.21 | 55.39 | 7.21 | 1.00 | 18.51 | 0.79 | ND | ND |
| 338-3 | | | | | | | | | |
| PES-08; | 11.40 | 3.40 | 54.39 | 8.05 | 1.07 | 18.54 | 0.74 | ND | ND |
| 338-10 | | | | | | | | | |
| PES-08; | 11.48 | 3.09 | 54.50 | 7.59 | 1.02 | 18.73 | 0.86 | ND | ND |
| 338-11 | | | | | | | | | |

ND—not detected

The ARA accumulation observed in derivative transgenic lines was markedly less (and did not translate into a measurable number in the GC output) than observed before (cf. ˜1.3% in examples 5, Table 6, and 7). This is because of lower amounts of DGLA produced in the parent PES-08 owing to Ps-FADdelta-8 being driven by the ApAMT1 promoter instead of the ApSAD2v1 promoter in this strain. PES-08; 333-9; PES-08; 334-11, and PES-08; 338-11 were banked as Phycoil strains PES-09, PES-10, and PES-11 respectively

We addressed the unavailability of sufficient DGLA in derivative lines by transforming it with a construct pPB0354 expressing another copy of Ps-FADdelta-8 driven by ApSAD2v1 promoter at A. protothecoides thiamine biosynthesis 4 (THI4) locus. We envisaged that an extra copy of Ps-FADdelta-8 driven by a stronger ApSAD2v1 promoter would boost DGLA to levels seen before (˜6%; examples 4 and 5 above) which will be used by Pt-FADdelta-5 enzyme as a substrate to produce substantial amounts of ARA seen earlier (˜1.5%; example 5 above) that can eventually be desaturated by one of the FADdelta-17 desaturases in PES-09, PES-10 or PES-11 strains. We also expressed a second copy of AtLPCAT1 and two new enzymes—a cytb5 from A. thaliana (AtCytb5-E AAC04491.1) and an LPAAT candidate from M. alpina (MaLPAAT; KAF9941528) to boost desaturation by various FAD desaturases and increase the incorporation of newly synthesized LCPUFAs into TAGs, respectively. The construct pPB0354 can be written as

pPB0354—ApTH14::ApSAD2v1p-PsFADd8-ApSAD2v13UTR:ApPGK1p-Neo-ApPGK3UTR:ApFBA1p-AtLPCAT1-ApFBA13UTR:ApAMT1p-SLC1-1(MaLPAAT)-ApPGH:ApAMT2v1p-AtCytB5E-ApHsp903UTR::ApTH14 The sequence of the transforming DNA construct pPB0354 is shown below in FIG. 25.

Relevant restriction sites in the construct are indicated in lowercase bold and are from 5′-3′ HindIII, XbaI, SpeI, PmeI, SnaBI, BmtI, HpaI, and HindIII, respectively. HindIII sites delimit the 5′ and 3′ ends of the transforming DNA. Underlined uppercase sequences represent genomic DNA from A. protothecoides PB5 that permit targeted integration of heterologous gene cassettes at the ApThi4 locus via homologous recombination. Proceeding from 5′ to 3′, A. protothecoides stearoyl ACP desaturase (ApSAD2v1) promoter (indicated as small case boxed text) drives the expression of codon-optimized P. salina FADdelta-8 (PsFADdelta-8) gene. The initiator ATG and terminator TGA for PsFADdelta-8 are indicated in uppercase italics, while the coding region is indicated in lowercase italics. The terminator region of the A. protothecoides stearoyl ACP desaturase (ApSAD2v1) gene is indicated by small capitals followed by the A. protothecoides phosphoglycerate kinase 1 (ApPGK1) promoter in lowercase, boxed text, driving expression of neomycin phosphotransferase II gene (Neo, codon-optimized for expression in A. protothecoides and encoding neomycin phosphotransferase II, thereby enabling the strain to grow on aminoglycoside antibiotic G418). The initiator ATG and terminator TGA for Neo are indicated in uppercase italics while the rest of the sequence is indicated in lowercase italics. The terminator region of the A. protothecoides phosphoglycerate kinase 1 (ApPGK1 terminator) is indicated by small capitals followed A. protothecoides fructose 1,6-bisphosphate aldolase (ApFBA1-1) promoter (indicated as small case boxed text) driving the expression of codon-optimized AtLPCAT1. The initiator ATG and terminator TGA for At-LPCAT1 are indicated by uppercase italics, while the coding region is indicated in lowercase italics. The ApFBA1-1 terminator region is indicated by small capitals followed A. protothecoides ammonium transporter 1 (ApAMT1) promoter, indicated as small case capitals driving the expression of a candidate LPAAT from Mortierella alpina (Ma-LPAAT). The initiator ATG and terminator TGA for Ma-LPAAT are indicated by uppercase italics, while the coding region is indicated in lowercase italics. The ApPGH terminator region is indicated by small capitals followed by A. protothecoides ammonium transporter 2 (ApAMT2v1) promoter (indicated as small case boxed text) driving the expression of codon-optimized A. thaliana cytochrome b5-E (At-Cytb5-E) gene. The initiator ATG and terminator TGA for At-Cytb5-E are indicated by uppercase italics, while the coding region is indicated with lowercase italics. The terminator region of the A. protothecoides heat shock protein 90 (ApHSP90) gene is indicated by small capitals followed by ApTHI4 genomic region indicated by underlined uppercase text. The final construct was sequenced to ensure correct reading frames and targeting sequences.

pPB354 was transformed into Phycoil strain PES-10 and the resulting primary transformants were selected on sucrose-containing growth media without thiamine and supplemented with G418. Single clonally purified colonies were grown under standard lipid production conditions in shake flasks. The resulting profiles from a set of representative derivative clones arising from transformations with the construct pPB0304 are shown in Table 12.

TABLE 12
Fatty acid profiles as a percentage of total fatty acids for parental strains (PES-8
and PES10) and representative derivative transgenic lines arising from the transformation
of PES-10 with plasmid pPB0354.

| Sample | C16:0 | C18:0 | C18:1n-9 | C18:2n-6 | C18:3n-3 | C20:2n-6 | C20:3n-6 | C20:4n-6 | C20:5n-3 |
|---|---|---|---|---|---|---|---|---|---|
| name | Palmitic | Stearic | Oleic | Linoleic | ALA | EDA | DGLA | ARA | EPA |
| PES-8 | 11.21 | 3.20 | 47.49 | 11.93 | 0.98 | 21.99 | 1.17 | — | — |
| Parent | | | | | | | | | |
| PES-10 | 11.23 | 11.42 | 3.22 | 53.96 | 0.86 | 20.93 | 0.54 | — | — |
| Parent | | | | | | | | | |
| PES-10; | 11.08 | 11.15 | 3.22 | 54.41 | 1.08 | 15.69 | 3.66 | ND | 1.24 |
| 354-1 | | | | | | | | | |
| PES-10; | 11.37 | 11.59 | 2.98 | 53.80 | 1.20 | 15.36 | 3.71 | ND | 1.30 |
| 354-2 | | | | | | | | | |

ND—not detected

As expected, a second copy of Ps-FADdelta-8 driven by a stronger ApSAD2v1 promoter in pPB0354 resulted in higher levels of DGLA in derivative transformants (3.66% and 3.71% DGLA in PES-10; 354-1 and PES-10; 354-2 vs 0.54% DGLA in PES-10 parent). Since the strain PES-10 already expresses Pt-FADdelta-5 and Pj-FADdelta-17, the enhanced DGLA in derivative lines was used as the substrate by Pt-FADdelta-5 to convert a proportion of DGLA into ARA which subsequently acted as a substrate for Pj-FADdelta-17 and resulted in the accumulation of 1.24% and 1.30% EPA in PES-10; 354-1 and PES-10; 354-2, respectively. Interestingly, no residual ARA was detected in any of the derivative strains suggesting that all of the available ARA was converted into EPA. The lipid assay on derivative strains along with controls was run a second time and similar results were obtained (data not shown). Since pPB0354 also expresses At-Cytb5 and Ma-LPAAT, conceivably either or both of these enzymes also positively modulate EPA accumulation in PES-10; 354-1 and PES-10; 354-2.

The work presented above clearly demonstrates that LCPUFA synthesis up to EPA and conceivably beyond is possible in our host organism. In ensuing experiments, we will use expression cassette optimization coupled with enzyme and strain evolution to significantly improve enzyme activity on various substrates (EDA, DGLA, and ARA) and enhance the production of various LCPUFAs in various ratios in our organism.

What is claimed is:

1. A process for the production of long-chain polyunsaturated fatty acids in recombinant Auxenochlorella protothecoides which comprises the following steps:
a) introducing a combination of at least one nucleic acid sequence which encodes elongases and at least one nucleic acid sequence which encodes desaturases into Auxenochlorella protothecoides to prepare the recombinant Auxenochlorella protothecoides; and
b) culturing the recombinant Auxenochlorella protothecoides to produce the long-chain polyunsaturated fatty acid.

2. The process of claim 1, wherein the long-chain polyunsaturated fatty acid is selected from the group consisting of eicosadienoic acid (EDA), dihomo-γ-linoleic acid (DGLA), arachidonic acid (ARA), and eicosapentaenoic acid (EPA).

3. The process of claim 1, wherein the long-chain polyunsaturated fatty acid is eicosapentaenoic acid (EPA).

4. The process of claim 1, wherein the elongase in the recombinant Auxenochlorella protothecoides is delta-9 elongase

5. The process of claim 4, wherein the delta-9 elongase is the elongase from the group consisting of Euglena gracilis, Isochrysis galbana, and Pavlova pinguis.

6. The process of claim 1, wherein the desaturase in the recombinant Auxenochlorella protothecoides is at least one gene selected from a group consisting of:
a) gene encoding delta-8 desaturase;
b) gene encoding delta-5 desaturase; and
c) gene encoding delta-17 desaturase.

7. The process of claim 6, wherein the delta-8 desaturase is a delta-8 desaturase from E. gracilis, Perkinus marinus, I. galbana, P. olseni, Mortierella sp. NVP85, Mortierella alpina, Diacronema lutheri, Pavlovales sp. CCMP2436, Pavlova salina, or Capsaspora owczarzaki.

8. The process of claim 6, wherein the delta-8 desaturases convert the EDA to DGLA.

9. The process of claim 6, wherein the delta-5 desaturase is a delta-5 desaturase from Phaeodactylum tricornutum, Dictyostelium discoideum, M. alpina, C. elegans, Oblongichytrium sp. SEK 347, Euglena gracilis, Parietochloris incisa, or Thalassiosira pseudonana CCMP1335.

10. The process of claim 6, wherein the delta-5 desaturase enzymes convert DGLA to ARA.

11. The process of claim 6, wherein the delta-17 desaturase is a delta-17 desaturases from Pythium aphanidermatum, Phytophthora sojae, Phytophthora ramorum, or Saprolegnia diclina.

12. The process of claim 6, wherein the delta-17 desaturase enzymes in the recombinant Auxenochlorella protothecoides convert ARA to EPA.

13. The process of claim 1, wherein recombinant Auxenochlorella protothecoides which further comprises
a) gene encoding lysophosphatidylcholine acyltransferase (LPCAT);
b) gene encoding lysophosphatidic acid acyltransferase (LPAAT);
c) gene encoding cytochrome b5 (Cytb5);
e) gene encoding choline phosphotransferase (CPT); and functional equivalents thereof.

14. A recombinant Auxenochlorella protothecoides for production of long-chain polyunsaturated fatty acid comprising:
a combination of at least one gene encoding elongase and at least one gene encoding a desaturase.

15. The recombinant Auxenochlorella protothecoides of claim 14, wherein the elongase is delta-9 elongase.

16. The recombinant Auxenochlorella protothecoides of claim 14, wherein the desaturase is at least one gene selected from a group consisting of:
a) gene encoding delta-8 desaturase;
b) gene encoding delta-5 desaturase; and
c) gene encoding delta-17 desaturase.

17. The recombinant Auxenochlorella protothecoides of claim 14, wherein recombinant Auxenochlorella protothecoides which further comprises
a) gene encoding lysophosphatidylcholine acyltransferase (LPCAT);
b) gene encoding lysophosphatidic acid acyltransferase (LPAAT);
c) gene encoding; cytochrome b5 (Cytb5);
e) gene encoding choline phosphotransferase (CPT); and functional equivalents thereof.

18. The recombinant Auxenochlorella protothecoides of claim 14, wherein recombinant Auxenochlorella protothecoides has an upregulated ACCase enzyme to boost levels of Malonyl CoA in the cytosol.

19. A recombinant nucleic acid comprising a coding sequence that encodes one or more selected from a group consisting of lysophosphatidylcholine acyltransferase (LPCAT), delta-9 elongase (delta-9FAE), delta-8 desaturase (FADdelta-8), delta-5 desaturase (FADdelta-5), delta-17 desaturases (FADdelta-17), lysophosphatidic acid acyltransferase (LPAAT), cytochrome b5 (Cytb5), choline phosphotransferase (CPT) and functional equivalents thereof.

20. The recombinant nucleic acid of claim 19, wherein the coding sequence is in operable linkage with a promoter.

21. A recombinant vector comprising a recombinant nucleic acid of claim 19.

22. An oil comprising long-chain polyunsaturated fatty acid produced by the recombinant Auxenochlorella protothecoides of claim 14.

23. The oil of claim 21, wherein the long-chain polyunsaturated fatty acid is selected from the group consisting of eicosadienoic acid (EDA), dihomo-γ-linoleic acid (DGLA), arachidonic acid (ARA) and eicosapentaenoic acid (EPA).

24. Composition comprising a recombinant Auxenochlorella protothecoides of claim 14, a culture thereof, or oil of claim 21.

25. The composition of claim 24, wherein the composition is a cosmetic composition, a food composition, a composition for a food additive, a feed composition, a composition for a feed additive, a pharmaceutical composition, a raw material composition for food, a raw material composition for feed, a raw material composition for pharmaceutics or a raw material composition for cosmetics.
